(19) United States
(12) Patent Application Publication
(10) Pub. No.: US 2022/0381790 A1
(43) Pub. Date: Dec. 1, 2022

(54) SELECTION AND BLOCKADE OF FERTILIZATION COMPETENT MALE AND FEMALE GAMETES

(71) Applicant: University of Virginia Patent Foundation, Charlottesville, VA (US)

(72) Inventors: Jeffrey J. Lysiak, Crozet (US); Kodimangalam S. Ravichandran, Earlysville (US)

(21) Appl. No.: 17/429,745

(22) PCT Filed: Feb. 11, 2020
(86) PCT No.: PCT/US20/17697

§ 371 Date: Aug. 10, 2021

Related U.S. Application Data
(60) Provisional application No. 62/803,702, filed on Feb. 11, 2019.

(51) Int. Cl. G01N 33/68; C12N 5/071

(52) U.S. Cl. CPC G01N 33/689; C12N 5/0612; G01N 2405/04; C12N 2500/36

(57) ABSTRACT

The presently disclosed subject matter relates to compositions and methods useful for assessing fertilization competency in male and female gametes. In some embodiments, methods, kits, and compositions for identifying, selecting, and functionally evaluating fertilization competent sperm cells are provided, wherein the methods, kits, and compositions employ a reagent that selectively binds phosphatidylserine (PS).

### CROSS REFERENCE TO RELATED APPLICATIONS

This application claims benefit of U.S. Provisional Application Ser. No. 62/803,702, filed Feb. 11, 2019, the disclosure of which is incorporated herein by reference in its entirety.

### GRANT STATEMENT

This invention was made with government support under Grant Nos. GM064709, MH096484, and HL120840 awarded by The National Institutes of Health. The government has certain rights in the invention.

### TECHNICAL FIELD

The presently disclosed subject matter relates to compositions and methods useful for assessing fertilization competency in male and female gametes. In some embodiments, methods, kits, and compositions for identifying, selecting, and functionally evaluating fertilization competent sperm cells are provided.

### BACKGROUND

Sexual reproduction requires a productive fusion between the haploid male and female gametes^{1, 2}. Prior to the fusion of the gametes, a critical step is the proper recognition between specific ligand(s) on the sperm and appropriate binding partner(s) on the egg. Recent studies both at the functional and structural levels have established a role for the sperm surface protein Izumol and the corresponding GPI-anchored receptor Juno on the oocyte, with blocking or loss of either protein affecting fertilization^{3, 4} Signaling downstream of Juno in oocytes is yet to be defined, as Juno is a GPI-anchored protein. Further, 3D structure studies^{5, 6} suggest that the Izumo:Juno interaction is unlikely to lead to fusion and, when Izumol was exogenously expressed on Cos-7 cells⁷, oocyte binding to these cells occurred but did not proceed to fusion. The tetraspanin family member CD9 on the oocyte has also been linked to mammalian fertilization⁸. CD9 has no known ligand and it is thought to modify the membrane curvature^{9, 10}. Thus, it has been suggested that other players on both sperm and the oocyte likely contribute toward gamete fusion (after the Izumo:Juno interaction)^{1, 2}. But, such players remain to be identified.

In accordance with the presently disclosed subject, using complimentary approaches on the oocyte and sperm, it is shown that phosphatidylserine (PtdSer or PS), exposed on viable sperm, is recognized by specific receptors located on the microvilli of the oocyte to promote sperm:egg fusion. The signaling pathway ELMO1/RAC1, downstream of the PtdSer receptors BAI1/3, also participates in this event. This pathway is also conserved in the fusion of sperm with myoblasts. Thus, in some aspects, the presently disclosed subject matter provides insights into the molecular mechanism of sperm:egg fusion.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

In some embodiments, the presently disclosed subject matter provides a method for detecting fertilization competent sperm cells. In some embodiments, the method comprises: (a) providing a sample comprising sperm cells from a subject; (b) mixing the sample with a reagent that selectively binds phosphatidylserine (PS) on the sperm cells; and (c) detecting living sperm cells bound to the reagent in the sample, whereby fertilization-competent sperm cells are detected. In some embodiments, the sample is formed by concentrating a semen sample from the subject.

In some embodiments, the subject is a human subject or a non-human animal subject. In some embodiments, the non-human animal subject is selected from the group consisting of bovine, equine, porcine, ovine, canine, feline, and avian.

In some embodiments, the reagent that selectively binds PS is selected from the group consisting of Annexin V, GST-BAI1-TSR, and derivatives thereof. In some embodiments, the reagent that selectively binds PS is labeled for detection.

In some embodiments, detecting living sperm cells comprises counting a total number of cells in the sample, counting apoptotic and/or necrotic cells, and counting cells bound to the reagent minus the apoptotic and/or dead cells. In some embodiments, counting apoptotic and/or necrotic cells comprises staining the sample with 7-aminoactinomycin D (7AAD), cleaved caspase 3 reagent, and similar apoptotic detection reagents. In some embodiments, identifying the sample as a fertile sample if an amount of living sperm cells bound to the reagent in the semen sample exceeds a predetermined number.

In some embodiments, the presently disclosed subject matter provides a method of, or kit for, detecting a fertilization competent oocyte in a sample. In some embodiments, the method comprises: (a) providing a sample comprising an oocyte from a subject; (b) mixing the sample with a reagent that selectively binds at least one phosphatidylserine (PS) receptor on the oocyte; and (c) detecting an oocyte bound to the reagent in the sample, whereby a fertilization-competent oocyte is detected. In some embodiments, the kit comprises a reagent that selectively binds at least one phosphatidylserine (PS) receptor; and instructional material for detecting a fertilization-competent oocyte in a sample.

In some embodiments, the subject is a human subject or a non-human animal subject. In some embodiments, the non-human animal subject is selected from the group consisting of bovine, equine, porcine, ovine, canine, feline, and avian.

In some embodiments, the reagent that selectively binds the at least one PS receptor comprises an antibody and/or modified bead, e.g., carboxylate modified bead, wherein the antibody and/or modified bead has an affinity for a PS receptor selected from the group consisting of CD36, BAI1, BAI3, Tim4, Mer-TK, and derivatives thereof. In some embodiments, the reagent that selectively binds the at least one PS receptor is labeled for detection.

In some embodiments, the presently disclosed subject matter provides a method of isolating fertilization competent sperm. In some embodiments, the method comprises (a) providing a sample comprising sperm cells from a subject; (b) mixing the sample with a reagent that selectively and/or reversibly binds phosphatidylserine (PS) on the sperm cells; and (c) isolating sperm cells bound to the reagent that selectively and/or reversibly binds PS, whereby fertilization competent sperm are isolated. In some embodiments, the sample is formed by concentrating a semen sample from the subject.

In some embodiments, the subject is a human subject or a non-human animal subject. In some embodiments, the non-human animal subject is selected from the group consisting of bovine, equine, porcine, ovine, canine, feline, and avian.

In some embodiments, the reagent that selectively and/or reversibly binds PS is selected from the group consisting of Annexin V, GST-BAI1-TSR, and derivatives thereof. In some embodiments, the reagent that selectively and/or reversibly binds PS is labeled for detection.

In some embodiments, isolating sperm bound to the reagent that selectively and/or reversibly binds PS comprises employing flow cytometry (FCM) or fluorescence-activated cell sorting (FACS). In some embodiments, the reagent that selectively and/or reversibly binds PS further comprises a capture moiety and isolating the sperm cells comprises isolating the capture moiety. In some embodiments, the capture moiety comprises a substrate and the reagent that selectively and/or reversibly binds PS is bound to the substrate. In some embodiments, the substrate comprises a microbead or a nanobead. In some embodiments, the method comprises eluting the sperm cells from the reagent that selectively and/or reversibly binds PS on the sperm cells.

In some embodiments, the presently disclosed subject matter provides a kit for detecting and/or isolating fertilization competent sperm cells in a sample. In some embodiments, the kit comprises a reagent that selectively and/or reversibly binds phosphatidylserine (PS); and instructional material for detecting and/or isolating fertilization-competent sperm cells in a sample.

In some embodiments, the reagent that selectively and/or reversibly binds PS is selected from the group consisting of Annexin V, GST-BAI1-TSR, and derivatives thereof. In some embodiments, the reagent that selectively and/or reversibly binds PS is detectably labeled. In some embodiment, the reagent selectively and reversibly binds PS.

In some embodiments, the kit comprises a reagent for staining apoptotic and/or necrotic cells. In some embodiments, the reagent for staining apoptotic and/or necrotic cells comprises 7AAD, cleaved caspase 3, or similar apoptotic detecting reagents. In some embodiments, the instruction material includes instructions for identifying the sample as a fertile sample if an amount of living sperm cells bound to the reagent in the semen sample exceeds a predetermined number.

In some embodiments, the kit comprises a reagent for concentrating sperm cells in a sample. In some embodiments, the kit comprises one or more reagents for carrying out flow cytometry (FCM) or fluorescence-activated cell sorting (FACS).

In some embodiments, the reagent that selectively and/or reversibly binds PS further comprises a capture moiety for use in isolating the sperm. In some embodiments, the capture moiety comprises a substrate and the reagent that selectively and/or reversibly binds PS is bound to the substrate. In some embodiments, the substrate comprises a microbead or a nanobead. In some embodiments, the kit further comprises a reagent and/or an apparatus for eluting the sperm cells from the reagent that selectively and/or reversibly binds PS.

In some embodiments, the presently disclosed subject matter provides a method of assessing fusion competency in sperm cells. In some embodiments, the method comprises: (a) providing sperm cells from a subject; (b) labeling the sperm cells with a reagent that can be subsequently tracked to assess fusion; (c) adding the sperm cells to a culture comprising myoblasts or other fusion competent cells; and (d) assessing fusion competency of the sperm cells by detecting the label transfer to myoblasts (or similar fusion-competent cells that can used as in vitro surrogates for oocytes) in the culture. In some embodiments, the sample is formed by concentrating a semen sample from the subject. In some embodiments, the reagent that can be subsequently tracked to assess fusion is a calcein or a DiI dye. In some embodiments, the fusion-competent cell culture comprises about 10,000 to about 50,000 fusion-competent cells.

In some embodiments, the subject is a human subject or a non-human animal subject. In some embodiments, the non-human animal subject is selected from the group consisting of bovine, equine, porcine, ovine, canine, feline, and avian.

In some embodiments, the method comprises adding a contrast agent to the culture. In some embodiments, the contrast agent comprises a Hoechst counterstaining agent.

In some embodiments, the presently disclosed subject matter provides a kit for assessing fusion competency in sperm cells. In some embodiments, the kit comprises a detectable reagent that selectively binds phosphatidylserine (PS) on the sperm cells; a culture comprising myoblasts (e.g. C2C12 mouse myoblast line or primary myoblasts), or other fusion competent cells (e.g. trophoblast cells); and instructional material for assessing fusion competency in the sperm cells by detecting the detectable reagent in the culture. In some embodiments, the sample is formed by concentrating a semen sample from the subject. In some embodiments, the fusion-competent cell culture comprises about 10,000 to about 50,000 fusion-competent cells.

In some embodiments, the reagent that selectively binds PS is selected from the group consisting of Annexin V, GST-BAI1-TSR, and derivatives thereof. In some embodiments, the detectable reagent comprises a reagent that can be subsequently tracked to assess fusion. In some embodiments, the reagent that can be subsequently tracked to assess fusion is calcein or a DiI dye.

In some embodiments, the kit comprises a contrast agent. In some embodiments, the contrast agent comprises a Hoechst counterstaining agent.

In some embodiments, the presently disclosed subject matter provides a method of blocking fertilization. In some embodiments, the method comprises providing a reagent that blocks PS binding between a sperm cell and an oocyte, and administering a composition comprising the reagent topically to a subject at a time of sexual intercourse. In some embodiments, the administering comprises applying the composition comprising the reagent to a contraceptive device. In some embodiments, the reagent that blocks PS binding between a sperm cell and an oocyte is selected from the group consisting of Annexin V, GST-BAI1-TSR, and derivatives thereof.

In some embodiments, the presently disclosed subject matter provides a composition comprising a reagent that blocks PS binding between a sperm cell and an oocyte; and a pharmaceutically acceptable carrier. In some embodiments, the reagent that blocks PS binding between a sperm cell and an oocyte is selected from the group consisting of Annexin V, GST-BAI1-TSR, and derivatives thereof.

In some embodiments, the presently disclosed subject matter provides a contraceptive device comprising a coating or lubricant comprising a reagent that blocks PS binding between a sperm cell and an oocyte. In some embodiments, the reagent that blocks PS binding between a sperm cell and an oocyte is selected from the group consisting of Annexin V, GST-BAI1-TSR, and derivatives thereof.

Accordingly, it is an object of the presently disclosed subject matter to provide methods and compositions for assessing fertilization competency in male and female gametes. This and other objects are achieved in whole or in part by the presently disclosed subject matter. Further, an object of the presently disclosed subject matter having been stated above, other objects and advantages of the presently disclosed subject matter will become apparent to those skilled in the art after a study of the following description, Figures, and Examples.

### BRIEF DESCRIPTION OF THE FIGURES

FIGS. 1A-1N depict that phosphatidylserine on live sperm is important for in vitro fertilization. FIG. 1A is a depiction of the mouse testis and epididymis. FIG. 1B depicts sperm from different regions of the epididymis that were allowed to swim/disperse in TYH+BSA medium, stained with Annexin V and Hoechst, and evaluated by microscopy. FIGS. 1C and 1D depict Annexin V staining of sperm. Asterisks denote sperm heads, and arrows midpiece. Scale bar, 20 μm. FIG. 1E depicts percentage of Annexin V+ sperm from the caput (n=9 mice), corpus (n=8 mice) and cauda (n=15 mice) epididymis, with each dot representing one mouse (6 independent experiments). *p<0.05, ***p<0.001 (One-way non-parametric ANOVA was followed by Kruskal Wallis test for multiple comparisons). FIGS. 1F and 1G depict sperm from cauda epididymis were incubated with 50 μg/ml GST only (FIG. 1F) or GST-BAI1-TSR (FIG. 1G), washed, fixed and visualized by GST immunofluorescence. Scale bar, 20 μm. FIG. 1H depicts snap shots of a movie depicting motility of live Annexin V+ (green) sperm (t: time in min). The trajectory of a single sperm is traced by a white dotted line. Scale bar, 30 μm. FIG. 1I is a schematic of the in vitro fertilization assay: cumulus oocyte complexes isolated from wild type (wt) super-ovulated females were incubated with caudal sperm previously capacitated, in the presence or absence of 10 μg/ml Annexin V, 50 μg/ml GST or 50 μg/ml GST-BAI1-TSR. The percentage of fertilized eggs (2-cell embryos) was evaluated after 24 h. FIG. 1J depicts multiple 2-cell embryos fertilization with control sperm (left panel, arrows), while fewer fertilized eggs were observed with Annexin V (right panel, arrows). Scale bar, 100 μm. FIGS. 1K and 1L depict Annexin V masking of PtdSer on sperm (FIG. 1K, n=4 experiments) or GST-BAI1-TSR (FIG. 1L, n=4 experiments) reduces fertilization. The total number of eggs analyzed is shown in parentheses. Each line represents one experiment and the matching experiments are shown (shape: square, circle, triangle, diamond; and color: (black square, red circle, green triangle, blue diamond)). Error bars are s.e.m. *p<0.05, **p<0.01 (Two tailed unpaired Student's t-test). FIGS. 1M and 1N depict greater unfertilized oocytes (asterisks) seen after competition with O-Phospho-L-Serine (bottom panel in FIG. 1M) compared to O-Phospho-D-Serine (top panel in FIG. 1M). Scale bar, 100 μm. Each line in FIG. 1N represents one experiment and matching experiments are shown with the same shape (square, circle, triangle, diamond) and color (n=4 experiments). Error bars are s.e.m. **p<0.01 (Two tailed unpaired Student's t-test).

FIGS. 2A-2L depict BAI1/3 and CD36 are expressed on oocytes and contribute to fertilization. FIG. 2A is a schematic for the assay to determine PtdSer recognition moieties on mouse oocytes. FIG. 2B depicts oocytes bound multiple untreated carboxylate beads within the microvillar region (left panel), but not the BAI1-TSR treated beads (right panel). Scale bar, 20 μm. FIG. 2C is a summary of 3 experiments using carboxylate binding oocytes. ***p<0.001 (Two tailed unpaired Student's t-test). FIG. 2D depicts PtdSer expression (qPCR) on cumulus-free Metaphase II oocytes from wild type female mice. (n=4 experiments). FIG. 2E depicts Concanavalin A (ConA) staining (marking microvilli) overlap with staining for BAI1/3 and CD36 on ZP-free oocytes. Scale bar, 20 μm. FIG. 2F shoes immunofluorescence (top panel) and immunohistochemistry (bottom panel) staining of mouse ovaries with BAI1/3 antibodies. Connexin-43 (green) marks follicular cells, ovarian follicles (dotted lines) are indicated. Arrows: oocytes in secondary or tertiary follicles; arrowheads: oocytes in primary follicles. Scale bar, 50 μm. FIG. 2G is a schematic of in vitro fertilization assays assessing the role of BAI1/3 and CD36. FIGS. 2H and 2I show multiple 2-cell embryos are observed in the control group (left panel, arrows) but reduced after CD36 and BAI1/3 antibody treatment (right panel). Scale bar, 100 μm. The compilation of data from 7 independent experiments is shown in FIG. 2I. Each dot represents one experiment, with matching experiments shown (shape and color, (black square, circle, triangle, diamond on the left; red or gray square, circle, triangle, diamond on the right)). sIgG, sheep IgG, mIgA, mouse IgA. **p<0.05 (Two tailed unpaired Student's t-test). FIG. 2J is a schematic of in vitro fertilization assays to test BAI1/3 and CD36 in sperm entry. Untreated or antibody-treated oocytes were loaded with DAPI, and mixed with sperm. The percentage of oocytes with decondensed sperm DNA was evaluated after 3 h. FIGS. 2K and 2L are representative images. A representative image of a fertilized oocyte with one decondensed sperm DNA is shown in FIG. 2K. Scale bar: 20 μm. Quantitation of oocytes with decondensed sperm DNA after blocking with CD36 and BAI1/3 antibodies is shown in FIG. 2L. Each dot represents one experiment (n=3 independent experiments) and the matching experiments are shown (shape and color (black square, circle, triangle on the left; red or gray square, circle, triangle on the right)). **p<0.01 (Two tailed unpaired Student t-test). Data are presented as mean±s.e.m.

FIGS. 3A-3K depict genetic testing of PtdSer receptors and cytoplasmic signaling in oocytes. FIG. 3A is a schematic of PtdSer receptors BAI1/3, the downstream ELMO-DOCK-RAC1 signaling pathway, and other receptors on oocytes. FIGS. 3B and 3C show that the PtdSer receptors Tim-4, BAI1 and Mer-TK participate in fertilization. ZP-intact oocytes from wt or Tim-4−/− mice (FIG. 3B) Mer-tk+/+ Bai1+/+, Mer-tk−/− Bai1+/+, or Mer-tk−/− Bai1+/− or Mer-tk−/− Bai1−/− (FIG. 3C) were mixed with wt sperm, and 2-cell embryos evaluated at 24 h. Fertilization index is the percentage of fertilized eggs from the experimental group divided by percentage of fertilized eggs from the control group (wt mice). Each dot represents one mouse (FIG. 3B: n=6 experiments including 17 wt mice and 9 Tim4−/− mice (black circles on the left; green or gray squares on the right, FIG. 3C: n=5 experiments including 15 wt mice, 6 Mer-tk−/− Bai1+/+ and 6 Mer-tk−/− Bai1−/−)(black circles on the left; blue or dark gray squares in the middle, red or light gray triangles on the right), total number of eggs (parentheses). *p<0.05 (FIG. 3B, Two tailed unpaired Student t-test), **p<0.01 (FIG. 3C, One-way ANOVA followed by Dunnet's multiple comparisons test). FIG. 3D depicts Elmo1 and Elmo2 mRNA on cumulus-free Metaphase II oocytes. FIG. 3E depicts intracellular ELMO1 in isolated Metaphase II (MII) ZP-free oocytes. Scale bar, 20 um. FIG. 3F is a schematic for generation of oocyte-specific Elmo1-deficient mice. FIG. 3G shows the percentage of fertilized eggs after incubation of control (Elmo1^{fl/fl}) or Elmo1-deficient (Ddr4-Cre/Elmo1^{fl/fl}) oocytes with wt sperm (n=6 independent experiments including 15 Elmo1^{fl/fl} mice and 12 Ddx4-Cre/Elmo1^{fl/fl} mice)(black circles on the left; red or gray squares on the right. Each dot represents 1 or 2 pooled mice. *p<0.05 (Two tailed unpaired Student's t-test). FIG. 3H is a schematic for the evaluation early sperm entry into oocytes via DAPI staining of decondensed sperm DNA. ZP-free wt oocytes were incubated with RAC1 inhibitor (EHT-1864, 80 AM) and loaded with DAPI. After several washes, sperm were added, and the presence of internalized sperm with decondensed nuclei was evaluated after 1 h. FIG. 3I depicts control oocytes displaying the decondensation of the sperm DNA incorporated into the oocyte (arrow), while the sperm tail has not yet been internalized. DAPI also highlights the oocyte chromosomes in anaphase II indicating the resumption of meiosis II. Scale bar, 20 μm. FIG. 3J depicts decreased percentage of oocytes with decondensed sperm DNA after RAC1 inhibition (n=4 independent experiments). Numbers in parentheses reflect total number of eggs scored. **p<0.01 (Two tailed unpaired Student's t-test). FIG. 3K depicts that sperm motility was not affected by RAC1 inhibition, p>0.05 (Two tailed unpaired Student's t-test). Data are presented as mean±s.e.m.

FIGS. 4A-4F depict sperm:myoblast fusion via PtdSer and the BAI3-ELMO2-RAC1 signaling axis. FIG. 4A shows that oocytes and myoblasts express similar molecules. FIG. 4B shows that Juno expression is readily detected on oocytes but not myoblasts. Bars represent mean±s.e.m. FIG. 4C is a schematic of the sperm:myoblast fusion assay. Caudal epididymal sperm were labeled with Calcein-AM (shaded more darkly or red) and co-cultured with murine C2C12 myoblasts. After 4 h, myoblasts were washed, stained with Hoechst dye (to stain nuclei) and the percentage of Calcein-AM⁺ myoblasts was evaluated by microscopy. FIG. 4D shows representative images depicting myoblasts that fuse with sperm to become Calcein-AM⁺ (3-10% per field) under control conditions (left panel), while this is greatly reduced when the sperm was pretreated with BAI1-TSR to mask PtdSer (middle panel) or after treatment of the myoblasts with the RAC1 inhibitor (right panel). Scale bar, 50 μm. FIG. 4E shows the detection of sperm inside the myoblasts. YFP⁺ sperm were co-incubated with C2C12 myoblasts for 4 h. Myoblasts were washed, fixed and stained by immunofluorescence with antibodies to YFP/GFP (green) and Izumol (pink). Phalloidin (red) and Hoechst (blue) were used to stain the actin cytoskeleton and DNA, respectively. On the left panel, the dense sperm nucleus contained within the phalloidin⁺ cytoplasm is shown on the cross-sectional plane. White arrows: sperm nucleus; green or gray arrow: sperm tail; dotted line: outline of the sperm head. Scale bar: 5 μm. FIG. 4F depicts reduced percentage of Calcein-AM⁺ myoblasts after masking of PtdSer on sperm using BAI1-TSR, antibody to BAI3, shRNA mediated knockdown of Elmo2 in myoblasts, treatment of myoblasts with the RAC1 inhibitor EHT-1864 or after pretreatment of myoblasts with cytoskeletal disruption via cytochalasin D (Cyto. D) or paraformaldehyde (PFA) fixation. (n=3 independent experiments for each condition except for RAC1 inhibitor, n=4 independent experiments). Bar charts show mean±s.e.m. *p<0.05, **p<0.001, ***p<0.0001 (One-way ANOVA followed by Dunnet's multiple comparisons test and two-tailed unpaired Student's t-test). Each dot represents one experiment.

FIGS. 5A-5F depict analysis of PtdSer exposure on sperm. FIG. 5A shows PtdSer exposure on sperm surface is enhanced during capacitation. Experimental design (left) showing the isolation and incubation of caudal sperm in non-capacitating (top, light gray or green) or capacitating medium containing CaCl₂), BSA and NaHCO₃ (bottom, dark gray or blue). In each mouse, one cauda (right) was used for the non-capacitating medium while the other cauda (left), was placed on capacitating medium. An increased percentage of Annexin V+ sperm is observed when capacitating medium was used (right). Each dot represents one mouse (n=8 mice) from 4 independent experiments. Mean s.e.m. *p=0.02 (Two tailed Mann Whitney non-parametric t-test). FIG. 5B shows that phosphatidylethanolamine (PtdEtn) was stained in caudal sperm using biotinylated Duramycin followed by streptavidin-Texas Red. Asterisks point to sperm head and arrows denote Duramycin+ sperm midpieces. Scale bar, 20 μm. FIGS. 5C and 5D show “fresh” caudal sperm (FIG. 5C) and sperm cultured for 24 h (FIG. 5D) were stained with Annexin V (red), fixed, permeabilized and stained with a Cleaved Caspase 3 (CC3 Cell Signaling, 9661S) Ab (green) by immunofluorescence. FIG. 5C: No CC3+ sperm were detected. FIG. 5D: Arrowhead: CC3+ sperm. Asterisks point to sperm head and arrows to Annexin V+ sperm midpiece (FIGS. 5C and 5D). Scale bar, 20 μm. FIGS. 5E and 5F show that to rule out effects on sperm vitality due to the buffers or the reagents used in in vitro fertilization assays (such as Annexin V, GST, or GST-BAI1-TSR), progressive motility was assessed. No significant differences were detected (n=4 experiments). Bar charts show mean±s.e.m. (FIG. 5E: p>0.05, two-tailed unpaired Student's t-test; FIG. 5F: p>0.05 one-way ANOVA).

FIGS. 6A-6J depict co-localization of PtdSer and Izumol on acrosome reacted sperm. Three different sperm are shown (FIGS. 6A-6C; FIGS. 6D-6F; FIGS. 6G-6J) with staining of PtdSer and Izumol on the equatorial region of the sperm head. FIGS. 6H-6J: higher magnification of boxed region in FIG. 6G. After capacitation of caudal sperm in TYH+BSA medium, the acrosome reaction was induced with the Ca⁺² ionophore A23187. Live sperm were stained with anti Izumol polyclonal antibody (which is exposed after acrosome reaction; ProSci, 8233) and Annexin V (conjugated with Alexa Fluor 568; red). Sperm were washed and fixed with 4% paraformaldehyde and placed on slides. Izumol antibody was detected with an anti-rabbit IgG-Alexa Fluor 488 (green, Invitrogen, A21208). Hoechst was used to stain nuclei. Samples were analyzed in an LSM 700 Zeiss Confocal microscope. Bar: 5 μm.

FIGS. 7A-7F depict analysis of mouse and human oocytes. FIG. 7A shows staining of CD9 and Juno on mouse Metaphase II ZP-free oocytes. FIG. 7B shows staining of BAI1/3 (top) and CD36 (bottom) on human oocytes by immunofluorescence. Arrows indicate residual cumulus cells. Scale bar, 20 μm. FIG. 7C shows staining of BAI1/3 in a biopsy of human ovary by immunohistochemistry (arrows indicate oocytes). Scale bar, 50 μm. FIG. 7D shows antibodies targeting either BAI1/3 or CD36 alone do not affect fertilization. Fertilization index was calculated as percentage of fertilized eggs from the antibody treated group (isotype controls or CD36 or BAI1/3 antibodies) divided by percentage of fertilized eggs from the untreated group. Numbers in parentheses are total number of eggs from 2 independent experiments (sIgG and BAI1/3 Ab) and from 5 independent experiments (mIgA and CD36 Ab). Bar charts show mean±s.e.m. sIgG: sheep IgG, mIgA: mouse IgA. (p>0.05; two-tailed unpaired Student's t-test). FIG. 7E shows oocytes from mice deficient in BAI1 show comparable fertilization to wt mice in the in vitro fertilization assays with wt sperm. Numbers in parentheses are total number of eggs, from 2 independent experiments (n=3 wt mice and n=3 BAI1−/− mice). Bar charts show mean±s.e.m. (p>0.05; two-tailed unpaired Student's t-test). FIG. 7F shows fertilization is potently blocked with CD9 or Juno antibodies. ZP-intact oocytes from wt mice were pre-incubated with CD9 or Juno antibodies and inseminated with wt sperm (Juno Ab: n=2 independent experiments and CD9 Ab: n=3 independent experiments. After 24 h, the percentage of fertilized eggs was evaluated (mean±s.e.m).

FIGS. 8A-8D depict transfer of the dye DiI from the sperm to the myoblasts. FIGS. 8A-8C show that C2C12 myoblasts were incubated with DiI-labeled sperm for 4 h. After the co-culture, multiple DiI+ myoblasts were observed (FIG. 8A). This was blocked when myoblasts were fixed with paraformaldehyde (PFA) before co-incubation with sperm (FIG. 8B), or by the addition of the RAC1 inhibitor (EHT-1864) (FIG. 8C). Scale bar, 50 μm. FIG. 8D shows the percentage of DiI+ myoblasts from 3 independent experiments is summarized. Bar charts show mean±s.e.m. **p<0.01 (One-way ANOVA followed by Dunnet's multiple comparisons test).

FIGS. 9A-9C show detection of sperm inside the myoblasts by electron microscopy. FIGS. 9A and 9B show electron microscopic images of sperm alone showing the typical structures of the midpiece with multiple mitochondria, the electron dense sperm nucleus, and the tail. Scale bar, 1 μm (FIG. 9A), 2 μm (FIG. 9B). FIG. 9C shows that after co-incubation of sperm with C2C12 myoblasts, the midpiece and tails of several sperm can be observed inside the myoblasts. Scale bar, 1 μm.

FIGS. 10A-10C depict primary myoblasts express PtdSer receptors and fuse with sperm. FIG. 10A shows primary myoblasts incubated with BAI1/3 (left) or CD36 (right) antibodies. Cells were fixed and sequentially stained with biotinylated antibodies, streptavidin-Texas Red, and Hoechst. Scale bar, 50 μm. FIG. 10B shows primary myoblasts co-cultured with Calcein-AM labeled sperm for 4 h. Arrows: transfer of the calcein dye from sperm to the myoblasts was visualized. Asterisks: residual sperm that has not fused. Scale bar, 50 μm. FIG. 10C shows primary myoblasts co-cultured with DiI-labeled sperm for 4 h. Arrows: transfer of the DiI dye from sperm to the myoblasts. Scale bar, 50 μm.

FIG. 11 depicts that bone marrow derived murine macrophages ‘phagocytose’ sperm. Calcein-AM labeled sperm was co-incubated with bone marrow derived macrophages. After 4 h, multiple sperm were observed within the macrophages. Please note that the dye transfer was not uniform within the cytoplasm as seen with myoblasts, and appears to be within endosomes. Asterisks: sperm heads. Scale bar, 50 μm.

FIGS. 12A and 12B depict that CD9 on myoblasts and participates in sperm:myoblast fusion. FIG. 12A shows that live C2C12 myoblasts (cultured in growth medium that does not support differentiation into myotubes) were stained with anti-CD9 antibody (clone KMC8), fixed, and stained with a secondary antibody (red) and the nuclei were stained with Hoechst (blue). Bar: 50 μm. FIG. 12B shows that CD9 antibody inhibits sperm:myoblast fusion. Calcein-AM labeled sperm were co-cultured with C2C12 myoblasts previously incubated with a CD9 antibody (clone KMC8). Fusion was evaluated as specified in FIGS. 4A-4F. Each dot represents on experiment (n=4 independent experiments). Mean±s.em.; *p<0.05 (Two tailed unpaired Student's t test).

FIG. 13 is a flow chart of an ART decision tree with the use of positive sperm for a more personalized treatment plan for a subject.

FIGS. 14A and 14B are digital images showing that PS is exposed on human sperm. Merged images of human sperm stained with annexin V (red) and DAPI (blue). Arrows indicate PS positive sperm (pink) from (FIG. 14A) a fertile male (normal semen analysis; proven paternity) and (FIG. 14B) from an infertile male (abnormal semen analysis; no offspring).

FIGS. 15A and 15B are a graph and a digital image, respectively, showing that human sperm fuse with mouse myoblasts. Following the procedure described herein below freshly isolated human ejaculated sperm fuse with C2C12 myoblasts (n=2). FIG. 15A) Fusion increased over time from 1 to 4 hr. FIG. 15B) Image showing fused myoblast (red; arrowheads) and stained sperm (red; arrows).

FIG. 16 is a schematic showing a sperm selection procedure.

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

### I. GENERAL CONSIDERATIONS

Infertility is a common problem in the United States. About 11% of women and 9% of men have reported problems with fertility. Currently, 20% of all infertility cases have no identifiable cause. With this high incidence of fertility issues, many couples resort to an Assisted Reproduction Technique (ART). In fact, 72,913 babies were born using ART in 2015 in the United States. This represents approximately 1 in 50 births in the United States, and in other countries such as Japan numbers are even greater, 1 in 20 births. The average cost of a single round of in vitro fertilization (IVF) in the United States is $12,400.00 and only 30% of IVF cycles produce a live birth. Thus, infertility affects large numbers of both females and males and contributes significantly to overall health care costs.

Fertilization requires the fusion of haploid gametes, sperm and egg, leading to creation of a new diploid organism. Although the process of fertilization has been studied for decades, relatively little is known about the molecules and mechanisms involved in sperm entry into the oocyte, a key step in fertilization. Understanding this step is critical not only at a fundamental scientific level but also to develop potential diagnostics and treatments for infertile couples. Currently, ART mainly involves IVF or IVF with intra-cytoplasmic sperm injection (IVF/ICSI). The development of additional and/or better tools for selecting appropriate gametes will improve IVF rates and the success of fertility treatments. This can be achieved using different approaches: 1) select or enrich for the “good” sperm; 2) identify the “fertile” oocytes, and 3) improve the conditions for the interaction of the oocytes and sperm.

Fertilization is essential for species survival. Although Izumol and Juno are critical for initial interaction between gametes, additional molecules necessary for sperm:egg fusion on both the sperm and the oocyte remain to be defined. In accordance with aspects of the presently disclosed subject matter, it is disclosed herein that phosphatidylserine (PtdSer or PS) is exposed on the head region of viable and motile sperm, with PtdSer exposure progressively increasing during sperm transit through the epididymis. Functionally, masking phosphatidylserine on sperm via three different approaches inhibits fertilization. On the oocyte, phosphatidylserine recognition receptors BAI1, CD36, Tim-4, and Mer-TK contribute to fertilization. Further, oocytes lacking the cytoplasmic ELMO1, or functional disruption of RAC1 (both of which signal downstream of BAI1/BAI3), also affect sperm entry into oocytes. Intriguingly, mammalian sperm could fuse with skeletal myoblasts, requiring PtdSer on sperm and BAI1/3, ELMO2, RAC1 in myoblasts. Collectively, these data identify phosphatidylserine on viable sperm and PtdSer recognition receptors on oocytes as players in sperm:egg fusion.

### II. DEFINITIONS

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. Mention of techniques employed herein are intended to refer to the techniques as commonly understood in the art, including variations on those techniques or substitutions of equivalent techniques that would be apparent to one of skill in the art. Thus, unless defined otherwise, all technical and scientific terms and any acronyms used herein have the same meanings as commonly understood by one of ordinary skill in the art in the field of the presently disclosed subject matter. Although any compositions, methods, kits, and means for communicating information similar or equivalent to those described herein can be used to practice the presently disclosed subject matter, particular compositions, methods, kits, and means for communicating information are described herein. It is understood that the particular compositions, methods, kits, and means for communicating information described herein are exemplary only and the presently disclosed subject matter is not intended to be limited to just those embodiments.

Following long-standing patent law convention, the terms “a”, “an”, and “the” refer to “one or more” when used in this application, including the claims. Thus, in some embodiments the phrase “a peptide” refers to one or more peptides.

The term “about”, as used herein to refer to a measurable value such as an amount of weight, time, dose (e.g., therapeutic dose), etc., is meant to encompass in some embodiments variations of ±20%, in some embodiments ±10%, in some embodiments ±5%, in some embodiments ±1%, in some embodiments ±0.5%, in some embodiments ±0.1%, and in some embodiments ±0.01% from the specified amount, as such variations are appropriate to perform the disclosed methods.

As used herein, the term “and/or” when used in the context of a list of entities, refers to the entities being present singly or in any and every possible combination and subcombination. Thus, for example, the phrase “A, B, C, and/or D” includes A, B, C, and D individually, but also includes any and all combinations and subcombinations of A, B, C, and D. It is further understood that for each instance wherein multiple possible options are listed for a given element (i.e., for all “Markush Groups” and similar listings of optional components for any element), in some embodiments the optional components can be present singly or in any combination or subcombination of the optional components. It is implicit in these forms of lists that each and every combination and subcombination is envisioned and that each such combination or subcombination has not been listed simply merely for convenience. Additionally, it is further understood that all recitations of “or” are to be interpreted as “and/or” unless the context clearly requires that listed components be considered only in the alternative (e.g., if the components would be mutually exclusive in a given context and/or could not be employed in combination with each other).

As used herein, the term “subject” refers to an individual (e.g., human, animal, or other organism) to be assessed, evaluated, and/or treated by the methods or compositions of the presently disclosed subject matter. Subjects include, but are not limited to, mammals (e.g., murines, simians, equines, bovines, porcines, canines, felines, and the like), and includes humans. As used herein, the terms “subject” and “patient” are used interchangeably, unless otherwise noted.

As used herein, the terms “effective amount” and “therapeutically effective amount” are used interchangeably and refer to the amount that provides a therapeutic effect, e.g., an amount of a composition that is effective to treat or prevent pathological conditions in a subject.

As used herein, the term “adjuvant” as used herein refers to an agent which enhances the pharmaceutical effect of another agent.

As used herein, amino acids are represented by the full name thereof, by the three letter code corresponding thereto, or by the one-letter code corresponding thereto, as indicated in the following table:

| | 3- | 1- | Functionally |
|---|---|---|---|
| | Letter | Letter | Equivalent |
| Full Name | Code | Code | Codons |
| Aspartic Acid | Asp | D | GAC GAU |
| Glutamic Acid | Glu | E | GAA GAG |
| Lysine | Lys | K | AAA AAG |
| Arginine | Arg | R | AGA AGG CGA CGC |
| | | | CGG CGU |
| Histidine | His | H | CAC CAU |
| Tyrosine | Tyr | Y | UAC UAU |
| Cysteine | Cys | C | UGC UGU |
| Asparagine | Asn | N | AAC AAU |
| Glutamine | Gin | Q | CAA CAG |
| Serine | Ser | s | ACG AGU UCA UCC |
| | | | UCG UCU |
| Threonine | Thr | T | ACA ACC ACG ACU |
| Glycine | Gly | G | GGA GGC GGG GGU |
| Alanine | Ala | A | GCA GCC GCG GCU |
| Valine | Val | V | GUA GUC GUG GUU |
| Leucine | Leu | L | UUA UUG CUA CUC |
| | | | CUG CUU |
| Isoleucine | He | I | AUA AUC AUU |
| Methionine | Met | M | AUG |
| Proline | Pro | P | CCA CCC CCG CCU |
| Phenylalanine | Phe | F | UUC UUU |
| Tryptophan | Trp | W | UGG |

The expression “amino acid” as used herein is meant to include both natural and synthetic amino acids, and both D- and L-amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage can be present or absent in the peptides of the presently disclosed subject matter.

The term “amino acid” is used interchangeably with “amino acid residue”, and can refer to a free amino acid and to an amino acid residue of a peptide. It will be apparent from the context in which the term is used whether it refers to a free amino acid or a residue of a peptide.

The term “antibody”, as used herein, refers to an immunoglobulin molecule which is able to specifically or selectively bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter may exist in a variety of forms. The term “antibody” refers to polyclonal and monoclonal antibodies and derivatives thereof (including chimeric, synthesized, humanized and human antibodies), including an entire immunoglobulin or antibody or any functional fragment of an immunoglobulin molecule which binds to the target antigen and or combinations thereof. Examples of such functional entities include complete antibody molecules, antibody fragments, such as Fᵥ, single chain Fᵥ, complementarity determining regions (CDRs), V_{L} (light chain variable region), V_{H} (heavy chain variable region), Fab, F(ab′)₂ and any combination of those or any other functional portion of an immunoglobulin peptide capable of binding to target antigen.

Antibodies exist, e.g., as intact immunoglobulins or as a number of well characterized fragments produced by digestion with various peptidases. Thus, for example, pepsin digests an antibody below the disulfide linkages in the hinge region to produce F(ab′)₂ a dimer of Fab which itself is a light chain joined to V_{H}-C_{H1} by a disulfide bond. The F(ab′)₂ may be reduced under mild conditions to break the disulfide linkage in the hinge region, thereby converting the F(ab′)₂ dimer into an Fab₁ monomer. The Fab₁ monomer is essentially an Fab with part of the hinge region (see Paul, 1993). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies.

An “antibody heavy chain”, as used herein, refers to the larger of the two types of polypeptide chains present in all antibody molecules.

An “antibody light chain”, as used herein, refers to the smaller of the two types of polypeptide chains present in all antibody molecules.

The term “single chain antibody” refers to an antibody wherein the genetic information encoding the functional fragments of the antibody are located in a single contiguous length of DNA. For a thorough description of single chain antibodies, see Bird et al., 1988; Huston et al., 1988).

The term “humanized” refers to an antibody wherein the constant regions have at least about 80% or greater homology to human immunoglobulin. Additionally, some of the nonhuman, such as murine, variable region amino acid residues can be modified to contain amino acid residues of human origin. Humanized antibodies have been referred to as “reshaped” antibodies. Manipulation of the complementarity-determining regions (CDR) is a way of achieving humanized antibodies. See for example, Jones et al., 1986; Riechmann et al., 1988, both of which are incorporated by reference herein. For a review article concerning humanized antibodies, see Winter & Milstein, 1991, incorporated by reference herein. See also U.S. Pat. Nos. 4,816,567; 5,482,856; 6,479,284; 6,677,436; 7,060,808; 7,906,625; 8,398,980; 8,436,150; 8,796,439; and 10,253,111; and U.S. Patent Application Publication Nos. 2003/0017534, 2018/0298087, 2018/0312588, 2018/0346564, and 2019/0151448, each of which is incorporated by reference in its entirety.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically-competent cells, or both. An antigen can be derived from organisms, subunits of proteins, whole cells, cellular components or cellular lysates.

As used herein, the term “antisense oligonucleotide” means a nucleic acid polymer, at least a portion of which is complementary to a nucleic acid which is present in a normal cell or in an affected cell. The antisense oligonucleotides of the presently disclosed subject matter include, but are not limited to, phosphorothioate oligonucleotides and other modifications of oligonucleotides. Methods for synthesizing oligonucleotides, phosphorothioate oligonucleotides, and otherwise modified oligonucleotides are well known in the art (see e.g., U.S. Pat. No. 5,034,506 to Summerton and Weller; Nielsen et al. (1991) Science 254:1497-1500). The term “antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence can be complementary to regulatory sequences specified on the coding strand of a DNA molecule encoding a protein, which regulatory sequences control expression of the coding sequences.

As used herein, the term “biologically active fragments” or “bioactive fragment” of a polypeptide encompasses natural or synthetic portions of the full-length protein that are capable of specific binding to their natural ligand or of performing the function of the protein.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. As used herein, the terms “complementary” or “complementarity” are used in reference to polynucleotides (i.e., a sequence of nucleotides) related by the base-pairing rules. For example, for the sequence “A-G-T”, is complementary to the sequence “T-C-A.”

The term “complex”, as used herein in reference to proteins, refers to binding or interaction of two or more proteins. Complex formation or interaction can include such things as binding, changes in tertiary structure, and modification of one protein by another, such as phosphorylation.

A “compound”, as used herein, refers to any type of substance or agent that is commonly considered a chemical, drug, or a candidate for use as a drug, as well as combinations and mixtures of the above. The term compound further encompasses molecules such as peptides and nucleic acids.

As used herein, a “derivative” of a compound refers to a chemical compound that can be produced from another compound of similar structure in one or more steps, such as in replacement of H by an alkyl, acyl, or amino group. Similarly, a “derivative” of a peptide (or of a polypeptide) is a compound that can be produced from or has a biological activity similar to a peptide (or a polypeptide) but that differs in the primary amino acid sequence of the peptide (or the polypeptide) to some degree. By way of example and not limitation, a derivative of a subject peptide of the presently disclosed subject matter is a peptide that has a similar although not identical primary amino acid sequence as the subject peptide (for example, has one or more amino acid substitutions) and/or that has one or more other modifications (e.g., N-terminal, C-terminal, and/or internal modifications) as compared to the subject peptide. Thus, the term “derivative” compasses the term “modified peptide” and vice versa, in the context of peptides. In some embodiments, a derivative of a peptide is a C-terminal amidated peptide. Derivative of antibodies can include but are not limited to antibody fragments, and chimeric, synthesized, humanized and human antibodies.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA can include introns.

As used herein, an “essentially pure” preparation of a particular protein or peptide is a preparation wherein at least about 95%, and preferably at least about 99%, by weight, of the protein or peptide in the preparation is the particular protein or peptide.

A “fragment” or “segment” is a portion of an amino acid sequence, comprising at least one amino acid of the amino acid sequence, or a portion of a nucleic acid sequence comprising at least one nucleotide. The terms “fragment” and “segment” are used interchangeably herein.

As used herein, a “functional” biological molecule is a biological molecule in a form in which it exhibits a property or activity by which it is characterized. A functional enzyme, for example, is one which exhibits the characteristic catalytic activity by which the enzyme is characterized.

The terms “formula” and “structure” are used interchangeably herein.

The term “identity” as used herein relates to the similarity between two or more sequences. Identity is measured by dividing the number of identical residues by the total number of residues and multiplying the product by 100 to achieve a percentage. Thus, two copies of exactly the same sequence have 100% identity, whereas two sequences that have amino acid deletions, additions, or substitutions relative to one another have a lower degree of identity. Those skilled in the art will recognize that several computer programs, such as those that employ algorithms such as BLAST (Basic Local Alignment Search Tool, Altschul et al. (1993) J Mol Biol 215:403-410) are available for determining sequence identity.

In some embodiments, “identity” can be expressed as a “percent identity”. As used herein, the phrase “percent identity” in the context of two nucleic acid or polypeptide sequences, refers to two or more sequences or subsequences that have in some embodiments 60%, in some embodiments 70%, in some embodiments 75%, in some embodiments 80%, in some embodiments 85%, in some embodiments 90%, in some embodiments 92%, in some embodiments 94%, in some embodiments 95%, in some embodiments 96%, in some embodiments 97%, in some embodiments 98%, in some embodiments 99%, and in some embodiments 100% nucleotide or amino acid residue identity, respectively, when compared and aligned for maximum correspondence, as measured using one of the following sequence comparison algorithms or by visual inspection. The percent identity exists in some embodiments over a region of the sequences that is at least about 50 residues in length, in some embodiments over a region of at least about 100 residues, and in some embodiments, the percent identity exists over at least about 150 residues. In some embodiments, the percent identity exists over the entire length of the sequences.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm disclosed in Smith & Waterman (1981) 2 Adv Appl Math 482-489; by the homology alignment algorithm disclosed in Needleman & Wunsch (1970) 48 J Mol Biol 443-453; by the search for similarity method disclosed in Pearson Pearson & Lipman (1988) Proc Natl Acad Sci USA 85:2444-2448; by computerized implementations of these algorithms (GAP, BESTFIT, FASTA, and TFASTA in the GCG® WISCONSIN PACKAGE®, available from Accelrys, Inc., San Diego, Calif., United States of America), or by visual inspection. See generally, Altschul et al. (1990) 215 J Mol Biol 403-410; Ausubel et al. (2002) Short Protocols in Molecular Biology, Fifth ed. Wiley, New York, N.Y., United States of America; and Ausubel et al. (2003) Current Protocols in Molecular Biology, John Wylie & Sons, Inc, New York, N.Y., United States of America.

One example of an algorithm that is suitable for determining percent sequence identity and sequence similarity is the BLAST algorithm, which is described in Altschul et al. (1990) 215 J Mol Biol 403-410. Software for performing BLAST analysis is publicly available through the website of the National Center for Biotechnology Information. This algorithm involves first identifying high scoring sequence pairs (HSPs) by identifying short words of length W in the query sequence, which either match or satisfy some positive valued threshold score T when aligned with a word of the same length in a database sequence. T is referred to as the neighborhood word score threshold. See generally, Altschul et al. (1990) 215 J Mol Biol 403-410. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=4, and a comparison of both strands. For amino acid sequences, the BLASTP program uses as defaults a wordlength (W) of 3, an expectation (E) of 10, and the BLOSUM62 scoring matrix. See Henikoff & Henikoff (1992) 89 Proc Natl Acad Sci USA 10915-10919.

In addition to calculating percent sequence identity, the BLAST algorithm also performs a statistical analysis of the similarity between two sequences (see e.g., Karlin & Altschul (1993) 90 Proc Natl Acad Sci USA 5873-5877). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in some embodiments less than about 0.1, in some embodiments less than about 0.01, and in some embodiments less than about 0.001.

The term “inhibit”, as used herein, refers to the ability of a compound or any agent to reduce or impede a described function or pathway. For example, inhibition can be by at least 10%, by at least 25%, by at least 50%, by at least 75%, by at least 80%, by at least 85%, by at least 90%, by at least 95%, by at least 97%, by at least 99%, or more.

As used herein, an “instructional material” includes a publication, a recording, a diagram, or any other medium of expression which can be used to communicate the usefulness of the presently disclosed subject matter in the kit for effecting alleviation of the various diseases or disorders recited herein. Optionally, or alternately, the instructional material can describe one or more methods of assessing fertilization competency of a gamete from a subject. The instructional material of the kit of the presently disclosed subject matter can, for example, be affixed to a container which contains a reagent of the presently disclosed subject matter or be shipped together with a container which contains the reagent. Alternatively, the instructional material can be shipped separately from the container with the intention that the instructional material and the reagent be used cooperatively by the recipient.

An “isolated” compound/moiety is a compound/moeity that has been removed from components naturally associated with the compound/moiety. For example, an “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

The term “modulate”, as used herein, refers to changing the level of an activity, function, or process. The term “modulate” encompasses both inhibiting and stimulating an activity, function, or process.

The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in an animal. In some embodiments, a pharmaceutically acceptable carrier is pharmaceutically acceptable for use in a human.

The term “polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides (e.g., a polypeptide of in some embodiments at least 50 amino acids, in some embodiments at least 75 amino acids, in some embodiments at least 100 amino acids, in some embodiments at least 200 amino acids, in some embodiments at least 300 amino acids, in some embodiments at least 500 amino acids, and in some embodiments more than 500 amino acids).

A peptide encompasses a sequence of 2 or more amino acids wherein the amino acids are naturally occurring or synthetic (non-naturally occurring) amino acids.

The term “linked” or like terms refers to a connection between two entities. The linkage can comprise a covalent, ionic, or hydrogen bond or other interaction that binds two compounds or substances to one another.

As used herein the term “peptidomimetic” refers to a chemical compound having a structure that is different from the general structure of an existing peptide, but that functions in a manner similar to the existing peptide, e.g., by mimicking the biological activity of that peptide. The term “modified peptide” encompasses a peptidomimetic. Peptidomimetics typically comprise naturally-occurring amino acids and/or unnatural amino acids, but can also comprise modifications to the peptide backbone. For example, a peptidomimetic can include one or more of the following modifications:

1. Peptides wherein one or more of the peptidyl —C(O)NR— linkages (bonds) have been replaced by a non-peptidyl linkage such as a —CH₂-carbamate linkage (—CH₂OC(O)NR—), a phosphonate linkage, a —CH₂-sulfonamide (—CH₂—S(O)₂NR—) linkage, a urea (—NHC(O)NH—) linkage, a —CH₂-secondary amine linkage, an azapeptide bond (CO substituted by NH), or an ester bond (e.g., depsipeptides, wherein one or more of the amide (—CONHR—) bonds are replaced by ester (COOR) bonds) or with an alkylated peptidyl linkage (—C(O)NR—) wherein R is C₁-C₆ alkyl;

2. Peptides wherein the N-terminus is derivatized to a —NRR1 group, to a —NRC(O)R group, to a —NRC(O)OR group, to a —NRS(O)₂R group, to a —NHC(O)NHR group where R and R1 are hydrogen or C₁-C₆ alkyl with the proviso that R and R1 are not both hydrogen;

3. Peptides wherein the C terminus is derivatized to —C(O)R2 where R2 is selected from the group consisting of C₁-C₆ alkoxy, and —NR3R4 where R3 and R4 are independently selected from the group consisting of hydrogen and C₁-C₄ alkyl;

4. Modification of a sequence of naturally-occurring amino acids with the insertion or substitution of a non-peptide moiety, e.g., a retroinverso fragment.

The term “permeability”, as used herein, refers to transit of fluid, cell, or debris between or through cells and tissues.

A “sample”, as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, gamete cells, or fluid of interest. A sample can also be obtained from cell or tissue culture.

By the terms “specifically binds” or “selectively binds”, as used herein, is meant a compound which recognizes and binds a specific target molecule, but does not substantially recognize or bind other molecules in a sample, or it means binding between two or more molecules as in part of a cellular regulatory process, where said molecules do not substantially recognize or bind other proteins in a sample.

The term “standard”, as used herein, refers to something used for comparison. For example, it can be a known standard agent or compound which is administered or added to a control sample and used for comparing results when measuring said compound in a test sample. Standard can also refer to an “internal standard”, such as an agent or compound which is added at known amounts to a sample and is useful in determining such things as purification or recovery rates when a sample is processed or subjected to purification or extraction procedures before a marker of interest is measured.

The term “symptom”, as used herein, refers to any morbid phenomenon or departure from the normal in structure, function, or sensation, experienced by the patient and indicative of disease. In contrast, a sign is objective evidence of disease. For example, a bloody nose is a sign. It is evident to the patient, doctor, nurse and other observers.

As used herein, the term “treating” includes prophylaxis of the specific disorder or condition, or alleviation of the symptoms associated with a specific disorder or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease.

A “therapeutic” treatment is a treatment administered to a subject who exhibits signs of pathology for the purpose of diminishing or eliminating those signs.

As used herein an “amino acid modification” refers in some embodiments to a substitution, addition, or deletion of an amino acid, and includes substitution with, or addition of, any of the 20 amino acids commonly found in human proteins, as well as unusual or non-naturally occurring amino acids such as but not limited to D-amino acids. Commercial sources of unusual amino acids include Sigma-Aldrich (Milwaukee, Wis., United States of America), ChemPep Inc. (Miami, Fla., United States of America), and Genzyme Pharmaceuticals (Cambridge, Mass., United States of America). Unusual amino acids can be purchased from commercial suppliers, synthesized de novo, or chemically modified or derivatized from naturally occurring amino acids. Amino acid modifications include linkage of an amino acid to a conjugate moiety, such as a hydrophilic polymer, acylation, alkylation, and/or other chemical derivatization of an amino acid. The term “modified peptide” encompasses any amino acid modification as described herein.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the presently disclosed subject matter are not limited to products of any of the specific exemplary processes listed herein.

Substitutions can be designed based on, for example, the model of Dayhoff et al. (in Atlas of Protein Sequence and Structure 1978, National Biomedical Research Foundation, Washington D.C., United States of America).

In some embodiments, an amino acid substitution is a conservative amino acid substitution. As used herein, the term “conservative amino acid substitution” is defined in some embodiments as exchanges within one of the following five groups:

I. Small aliphatic, nonpolar or slightly polar residues: Ala, Ser, Thr, Pro, Gly;

II. Polar, charged residues and their amides: Asp, Asn, Glu, Gln, His, Arg, Lys;

III. Large, aliphatic, nonpolar residues: Met Leu, Ile, Val, Cys

IV. Large, aromatic residues: Phe, Tyr, Trp

Conservative substitutions are likely to be phenotypically silent. Typically seen as conservative substitutions are the replacements, one for another, among the aliphatic amino acids Ala, Val, Leu, and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr. Guidance concerning which amino acid changes are likely to be phenotypically silent are found in Bowie et al. (1990) Science 247:1306-1310.

For example, the hydropathic index of amino acids may be considered (Kyte & Doolittle (1982) J Mol Biol 157:105-132). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle (1982) J Mol Biol 157:105-132), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, the use of amino acids whose hydropathic indices are within +/−2 is preferred, within +/−1 are more preferred, and within +/−0.5 are even more preferred.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Replacement of amino acids with others of similar hydrophilicity is preferred.

Other considerations include the size of the amino acid side chain. For example, in some embodiments an amino acid with a compact side chain, such as glycine or serine, would not be replaced with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet, or reverse turn secondary structure has been determined and is known in the art (see e.g., Chou & Fasman (1974) Biochemistry 13:222-245; Chou & Fasman (1978) Ann Rev Biochem 47: 251-276; Chou & Fasman (1979) Biophys J 26:367-384).

Based on such considerations and extensive empirical study, tables of conservative amino acid substitutions have been constructed and are known in the art. By way of example and not limitation, the following substitutions can be made: arginine and lysine; glutamate and aspartate; serine and threonine; glutamine and asparagine; and valine, leucine, and isoleucine. Alternatively, Table 1 lists exemplary conservative amino acid substitutions.

TABLE 1
Exemplary Conservative Amino Acid Substitutions

| Amino Acid | Possible Substitution(s) |
|---|---|
| Ala (A) | Leu, Ile, Val |
| Arg (R) | Gln, Asn, Lys |
| Asn (N) | His, Asp, Lys, Arg, Gln |
| Asp (D) | Asn, Glu |
| Cys (C) | Ala, Ser |
| Gln (Q) | Glu, Asn |
| Glu (E) | Gln, Asp |
| Gly (G) | Ala |
| His (H) | Asn, Gln, Lys, Arg |
| Ile (I) | Val, Met, Ala, Phe, Leu |
| Leu (L) | Val, Met, Ala, Phe, Ile |
| Lys (K) | Gln, Asn, Arg |
| Met (M) | Phe, Ile, Leu |
| Phe (F) | Leu, Val, Ile, Ala, Tyr |
| Pro (P) | Ala |
| Ser (S) | Thr |
| Thr (T) | Ser |
| Trp (W) | Phe, Tyr |
| Tyr (Y) | Trp, Phe, Thr, Ser |
| Val (V) | Ile, Leu, Met, Phe, Ala |

In some embodiments, another consideration for amino acid substitutions include whether or not the residue is located in the interior of a protein or is solvent exposed. For interior residues, conservative substitutions can include in some embodiments: Asp and Asn; Ser and Thr; Ser and Ala; Thr and Ala; Ala and Gly; Ile and Val; Val and Leu; Leu and Ile; Leu and Met; Phe and Tyr; Tyr and Trp. For solvent exposed residues, conservative substitutions can include in some embodiments: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr. Various matrices have been constructed to assist in selection of amino acid substitutions, such as the PAM250 scoring matrix, the Dayhoff matrix, the Grantham matrix, the McLachlan matrix, the Doolittle matrix, the Henikoff matrix, the Miyata matrix, the Fitch matrix, the Jones matrix, the Rao matrix, the Levin matrix, and the Risler matrix (summarized in, for example, Johnson & Overington (1993) J Mol Biol 233:716-738; see also the PROWL resource available at the website of The Rockefeller University, New York, N.Y., United States of America).

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded peptide sequence are well known and a matter of routine experimentation for the skilled artisan, for example by the technique of site-directed mutagenesis or by synthesis and assembly of oligonucleotides encoding an amino acid substitution and splicing into an expression vector construct.

### III. METHODS, COMPOSITIONS, DEVICES, AND KITS

Fertilization has been studied for decades, yet the understanding of the molecular events responsible for the incorporation of the sperm into the oocyte is incomplete. Conventional semen analysis, which simply counts sperm number and assesses sperm motility and morphology, is a testament to this lack of understanding, and leading experts agree that there exists little evidence that correlates sperm fertility with these factors. Thus, current practices are to label men who have “normal” sperm numbers and good motility as “fertile”, regardless of their sperm's actual ability to fertilize an oocyte. In these instances, the burden of fertility treatment is passed to the woman, where health care costs can skyrocket into the tens of thousands.

The diagnostic weakness of a semen analysis means that, of the men undergoing a fertility evaluation, up to 40% will have an unknown cause. In the United States, an estimated 7 million couples per year seek infertility care and a male factor contributes to 50% of cases. Often, the results of the semen analysis determine the assisted reproductive technology (ART) treatment pathway (e.g., intra-uterine insemination (IUI), in vitro fertilization (IVF), or intra-cytoplasmic sperm injection (ICSI)). For these procedures, sperm are selected based on the presence of motility and morphology parameters that do not predict fertilization, or the use of staining techniques that render the sperm unusable. Better tools to assess sperm fertility are needed to improve the quality of information that millions of couples currently lack when determining their ART treatments. Moreover, male fertility is a barometer of men's health with clear associations to diabetes, heart disease, and mortality.

More information about male infertility is needed, such as the need for more specific fertility tests. In some aspects the presently disclosed subject matter provides tests that provide information about the potential for a sperm to fuse with an egg. In some aspects, the presently disclosed subject matter uses expression of a novel biomarker, phosphatidylserine (PS), on sperm, to characterize the potential for sperm to fuse with an egg. In some aspects, the presently disclosed subject matter provides:

Detection of PS on human sperm: Conventional semen analysis does not provide insight about the molecule(s) on sperm essential for fusion with the oocyte. Assay for PS on a sperm's surface in accordance with the presently disclosed subject matter demonstrate the feasibility of using PS as a biomarker of fertility. PS is already expressed on the surface of sperm from the cauda epididymis and human ejaculated sperm, making it a useful biomarker, unlike Izumol that only is expressed to the cell surface after the acrosome reaction that occurs in the female reproductive tract.

Aiding in assisted-reproductive technique (ART) decisions: Determining if sperm express PS guides infertility experts to better decisions on the ART specific for the individual. If sperm possess PS and are high in numbers, intrauterine insemination (IUI) may be the applicable ART. Knowing this information ultimately saves couples money and emotional distress.

Companion diagnostic kit: Companion diagnostic or at-home test kits are a rapidly growing market. A notable clinical barrier in the field is the reticence of men to visit a doctor for potential fertility issues. In some embodiments, the presently disclosed subject matter provides an at-home kit that test for PS positive sperm in a disposable “app” driven test kit. This kit substantially reduces patient anxiety and confers to men the option of utilizing an at-home diagnostic that can help to guide their future healthcare decisions more quickly and effectively for, ultimately, improved fertility outcomes.

Aiding in assisted-reproductive technique (ART) decisions: Determining the level of PS expressing sperm guides infertility experts to better decisions on the ART specific for the individual (see FIG. 13). If sperm possess PS and are high in numbers, intrauterine insemination (IUI) may be the applicable ART. Knowing this information can ultimately save couples money and emotional distress.

Selection of PS-positive sperm: Further evidence about the role of PS in human sperm fusion provides for selection of the PS-positive sperm that then can be used directly in the appropriate ART. An assay that allows for the enrichment or selection of “more fertile” sperm to improve the efficiency of in-vitro fertilization is also provided.

Contraception: Since PS is directly involved in sperm:egg fusion, i.e. fertilization, PS-blocking agents as a non-hormonal, reversible, contraceptive compositions are provided.

Representative sequences for GST-BAI1-TSR (Park et al. 2007. Nature 450:430-434):

GST (SEQ ID NO: 1):
MSPILGYWKIKGLVQPTRLLLEYLEEKYEEHLYERDEGDKWRNKKFELG
LEFPNLPYYIDGDVKLTQSMAIIRYIADKHNMLGGCPKERAEISMLEGA
VLDIRYGVSRIAYSKDFETLKVDFLSKLPEMLKMFEDRLCHKTYLNGDH
VTHPDFMLYDALDVVLYMDPMCLDAFPKLVCFKKRIEAIPQIDKYLKSS
KYIAWPLQGWQATFGGGDHPPK
BAI1-TSR (residues 202-585)
(SEQ ID NO: 2)
SSHPCGIMQTPCACLGGDVGDPASSPLVPRGDVCLRDGVAGGPENCLTS
LTQDRGGHGSAGGWKLWSLWGECTRDCGGGLQTRTRTCLPTLGVEGGGC
EGVLEEGRLCNRKACGPTGRSSSRSQSLRSTDARRREEFGDELQQFGFP
SPQTGDPAAEEWSPWSVCSSTCGEGWQTRTRFCVSSSYSTQCSGPLREQ
RLCNNSAVCPVHGAWDEWSPWSLCSSTCGRGFRDRTRTCRPPQFGGNPC
EGPEKQTKFCNIALCPGRAVDGNWNEWSSWSTCSASCSQGRQQRTRECN
GPSYGGAECQGHWVETRDCFLQQCPVDGKWQAWASWGSCSVTCGGGSQR
RERVCSGPFFGGAACQGPQDEYRQCGAQRCPEPHEICDEDN

In some embodiments, the presently disclosed subject matter provides derivatives of GST-BAI1-TSR that comprise isolated and purified peptides that comprise an amino acid sequence selected from the group consisting of SEQ ID NOs: 1 and 2, a fragment thereof, a peptide having an amino acid sequence that is approximately 95% identical to the sequence of any one of SEQ ID NOs: 1 and 2, a fragment thereof, and substantially homologous amino acid sequences of any of the foregoing sequences. In some embodiments, the amino acid sequence comprises at least one modification selected from the group consisting of an amino acid deletion, an amino acid addition, an amino acid substitution, and combinations thereof.

### III.A. METHODS AND KITS FOR DETECTING FERTILIZATION COMPETENT SPERM CELLS

In some embodiments, the presently disclosed subject matter provides methods for detecting fertilization competent sperm cells. By “fertilization competent” it is meant a sperm capable of fusion with an egg and the subsequent fertilization of the egg by the sperm. In some embodiments, the method comprises: (a) providing a sample comprising sperm cells from a subject; (b) mixing the sample with a reagent that selectively binds phosphatidylserine (PS) on the sperm cells; and (c) detecting living sperm cells bound to the reagent in the sample, whereby fertilization-competent sperm cells are detected.

In some embodiments, the subject is a human subject or a non-human animal subject. In some embodiments, the non-human animal subject is selected from the group consisting of bovine, equine, porcine, ovine, canine, feline, and avian.

In some embodiments, the sample is formed by concentrating a semen sample from the subject. Approaches, reagents, and/or devices for concentrating a semen sample are disclosed elsewhere herein and include, but are not limited to, enriching sperm in the sample using an isolation gradient. Indeed, any suitable approach, reagent, and/or devices for concentrating sperm in a sample as would be apparent to one of ordinary skill in the art upon a review of the instant disclosure is provided in accordance with the presently disclosed subject matter. The sperm swim-up technique is another method for enriching sperm that is recognized in the art. Briefly, the sperm swim-up technique involves liquefaction of a semen sample followed by centrifugation of the semen sample and discarding of the supernatant. The pellet is suspended in a pre-warmed medium, such as Ham's F-10 culture medium, and then centrifuged again. Medium is gently over-layered on the resulting pellet in the tube, which is sealed. The tube is inclined at 45 degrees and kept at 37° C. for 60-90 minutes under carbon dioxide, such as 5% CO₂. A supernatant containing actively motile sperms is removed by sterile techniques, such as via a sterile Pasteur pipette. See Jameel, T., J Pak Med Assoc. 2008 February; 58(2):71-4; Volpes et al., J Assist Reprod Genet. 2016 June; 33(6): 765-770.

In some embodiments, the reagent that selectively binds PS is chosen from the group consisting of Annexin V, GST-BAI1-TSR, an anti-PS antibody, and derivatives thereof. In some embodiments, the reagent that selectively binds PS is labeled for detection (such as fluorescence, biotinylation, etc.).

In some embodiments, detecting living sperm cells comprises counting a total number of cells in the sample, counting apoptotic and/or necrotic cells, and counting cells bound to the reagent minus the apoptotic and/or dead cells. In some embodiments, counting apoptotic and/or necrotic cells comprises staining the sample with 7AAD, cleaved caspase 3 reagent, and similar apoptotic detection reagents. In some embodiments, the method comprises identifying the sample as a fertile sample if an amount of living sperm cells bound to the reagent in the semen sample exceeds a predetermined number. By way of example and not limitation, fertile samples contain at least about ˜50% PS+ cells. By way of elaboration and not limitation, ˜50% percent PS positive sperm were detected from sperm samples from the cauda epididymis of normal fertile male mice.

In some embodiments, the presently disclosed subject matter provides a kit for detecting and/or isolating fertilization competent sperm cells in a sample. In some embodiments, the kit comprises a reagent that selectively binds phosphatidylserine (PS); and instructional material for detecting and/or isolating fertilization-competent sperm cells in a sample.

In some embodiments, the reagent that selectively binds PS is chosen from the group consisting of Annexin V, GST-BAI1-TSR, an anti-PS antibody, and derivatives thereof. In some embodiments, the reagent that selectively binds PS is detectably labeled, (such as fluorescence, biotinylation, etc.).

In some embodiments, the kit comprises a reagent for staining apoptotic and/or necrotic cells. In some embodiments, the reagent for staining apoptotic and/or necrotic cells comprises 7AAD, cleaved caspase 3, or similar apoptotic detecting reagents. In some embodiments, the instruction material includes instructions for identifying the sample as a fertile sample if an amount of living sperm cells bound to the reagent in the semen sample exceeds a predetermined number. By way of example and not limitation, fertile samples contain ˜50% PS+ cells.

In some embodiments, the kit comprises a reagent and/or device for concentrating sperm cells in a sample. Reagents for concentrating a semen sample are disclosed elsewhere herein and include, but are not limited to, reagents and/or devices enriching sperm in the sample using an isolation gradient. Indeed, any suitable reagent, device, and/or approach for concentrating sperm in a sample as would be apparent to one of ordinary skill in the art upon a review of the instant disclosure is provided in accordance with the presently disclosed subject matter. The sperm swim-up technique is another method for enriching sperm, described elsewhere herein.

In some embodiments, the presently disclosed subject matter provides a method of, or kit for, detecting a fertilization competent oocyte in a sample. In some embodiments, the method comprises: (a) providing a sample comprising an oocyte from a subject; (b) mixing the sample with a reagent that selectively binds at least one phosphatidylserine (PS) receptor on the oocyte; and (c) detecting an oocyte bound to the reagent in the sample, whereby a fertilization-competent oocyte is detected. In some embodiments, the kit comprises a reagent that selectively binds at least one phosphatidylserine (PS) receptor; and instructional material for detecting a fertilization-competent oocyte in a sample.

In some embodiments, the subject is a human subject or a non-human animal subject. In some embodiments, the non-human animal subject is selected from the group consisting of bovine, equine, porcine, ovine, canine, feline, and avian.

In some embodiments, the reagent that selectively binds the at least one PS receptor comprises an antibody and/or modified bead, e.g., carboxylate modified bead, wherein the antibody and/or modified bead has an affinity for a PS receptor selected from the group consisting of CD36, BAI1, BAI3, Tim4, Mer-TK, and derivatives thereof. In some embodiments, the reagent that selectively binds the at least one PS receptor is labeled for detection.

In some embodiments, the method further comprises using the results of the gamete analysis to determine an assisted reproductive technology (ART) treatment pathway (e.g., intra-uterine insemination (IUI), in vitro fertilization (IVF), or intra-cytoplasmic sperm injection (ICSI)) for a subject. In some embodiments, the instruction material includes instructions for determining an assisted reproductive technology (ART) treatment pathway (e.g., intra-uterine insemination (IUI), in vitro fertilization (IVF), or intra-cytoplasmic sperm injection (ICSI)) for a subject. Thus, the presently disclosed subject matter provides tools to assess sperm fertility to improve the quality of information that millions of couples currently lack when determining their ART treatments. Moreover, male fertility is a barometer of men's health with clear associations to diabetes, heart disease, and mortality. Thus, in some embodiments, the method further comprises discussing and/or suggesting additional health care interventions to a male patient.

### III.B. METHODS AND KITS FOR ISOLATING FERTILIZATION COMPETENT SPERM CELLS

In some embodiments, the presently disclosed subject matter provides methods of isolating fertilization competent sperm. In some embodiments, the method comprises: (a) providing a sample comprising sperm cells from a subject; (b) mixing the sample with a reagent that selectively and/or reversibly binds phosphatidylserine (PS) on the sperm cells; and (c) isolating sperm cells bound to the reagent that selectively and/or reversibly binds PS, whereby fertilization competent sperm are isolated.

In some embodiments, the subject is a human subject or a non-human animal subject. In some embodiments, the non-human animal subject is selected from the group consisting of bovine, equine, porcine, ovine, canine, feline, and avian.

In some embodiments, the sample is formed by concentrating a semen sample from the subject. Approaches, reagents, and/or devices for concentrating a semen sample are disclosed elsewhere herein and include, but are not limited to, approaches, reagents, and/or devices enriching sperm in the sample using an isolation gradient. Indeed, any suitable reagent, device, and/or approach for concentrating sperm in a sample as would be apparent to one of ordinary skill in the art upon a review of the instant disclosure is provided in accordance with the presently disclosed subject matter.

In some embodiments, the reagent that selectively and/or reversibly binds PS on the sperm cells is chosen from the group consisting of Annexin V, GST-BAI1-TSR, an anti-PS antibody, and derivatives thereof. In some embodiments, the reagent that selectively and/or reversibly binds PS on the sperm cells is detectably labeled (such as fluorescence, biotinylation, etc.).

In some embodiments, isolating sperm bound to the reagent that that selectively binds PS comprises employing flow cytometry (FCM) or fluorescence-activated cell sorting (FACS). Any suitable FCM or FACS technique as would be apparent to one of ordinary skill in the art upon a review of the instant disclosure is provided in accordance with the presently disclosed subject matter. By way of example and not limitation, sperm cells can be labeled with Annexin V as described herein and FACS performed.

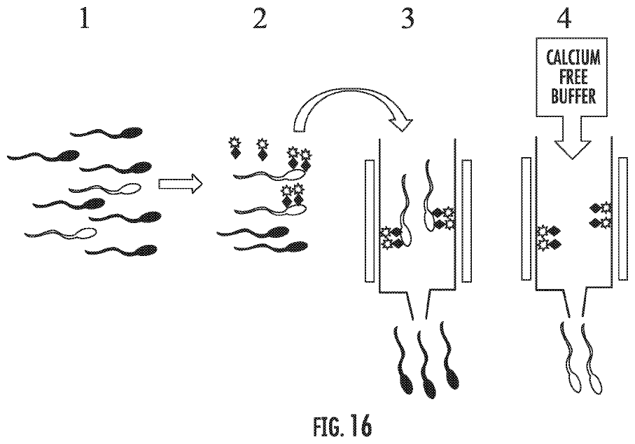

In some embodiments, the reagent that selectively and/or reversibly binds PS further comprises a capture moiety and isolating the sperm cells comprises isolating the capture moiety. In some embodiments, the capture moiety comprises a substrate. The reagent that binds PS can be reversible. Thus, in some embodiments, the reagent that selectively and/or reversibly binds PS is bound to the substrate. In some embodiments, the substrate comprises a microbead or a nanobead. In some embodiments, the method further comprises eluting the sperm cells from the reagent that selectively and/or reversibly binds PS on the sperm cells. By way of example and not limitation, a spermatozoa-microbeads suspension is loaded on a column containing a coated cell-friendly matrix containing iron balls, which was fitted in a magnet (MiniMACS; Miltenyi Biotec). The power of magnetic field is measured as about 0.5 Tesla between the poles and up to about 1.5 Tesla within the iron globes of the column. The fraction containing the PS+ sperm (due to binding to Annexin V beads) is retained in the column, and the then sperm will be eluted with a Ca⁺⁺ free buffer. As the binding of Annexin V to PS is Ca⁺⁺ dependent, the Ca⁺⁺ free buffer releases the PS+ sperm from the Annexin microbeads. See Example 8 and FIG. 16.

In some embodiments, the presently disclosed subject matter provides a kit for detecting and/or isolating fertilization competent sperm cells in a sample. In some embodiments, the kit comprises a reagent that selectively and/or reversibly binds phosphatidylserine (PS); and instructional material for detecting and/or isolating fertilization-competent sperm cells in a sample.

In some embodiments, the reagent that selectively and/or reversibly binds PS is chosen from the group consisting of Annexin V, GST-BAI1-TSR, an anti-PS antibody, and derivatives thereof. In some embodiments, the reagent that selectively and/or reversibly binds PS is detectably labeled (such as fluorescence, biotinylation, etc.).

In some embodiments, the kit comprises a reagent for staining apoptotic and/or necrotic cells. In some embodiments, the reagent for staining apoptotic and/or necrotic cells comprises 7AAD, cleaved caspase 3, or similar apoptotic detecting reagents. In some embodiments, the instruction material includes instructions for identifying the sample as a fertile sample if an amount of living sperm cells bound to the reagent in the semen sample exceeds a predetermined number.

In some embodiments, the kit comprises a reagent for concentrating sperm cells in a sample. Approaches, reagents, and/or devices for concentrating a semen sample are disclosed elsewhere herein and include, but are not limited to, approaches, reagents, and/or devices enriching sperm in the sample using an isolation gradient. Indeed, any suitable reagent, device, and/or approach for concentrating sperm in a sample as would be apparent to one of ordinary skill in the art upon a review of the instant disclosure is provided in accordance with the presently disclosed subject matter.

In some embodiments, the kit comprises one or more reagents for carrying out flow cytometry (FCM) or fluorescence-activated cell sorting (FACS). In some embodiments, the reagent that selectively and/or reversibly binds PS further comprises a capture moiety for use in isolating the sperm. In some embodiments, the capture moiety comprises a substrate and the reagent that selectively and/or reversibly binds PS is bound to the substrate. In some embodiments, the substrate comprises a microbead or a nanobead. In some embodiments, the kit further comprises a reagent and an apparatus (e.g., buffer and column) for eluting the sperm cells from the reagent that selectively and/or reversibly binds PS.

In some embodiments, the reagent that selectively and/or reversibly binds PS on the sperm cells is chosen from the group consisting of Annexin V, GST-BAI1-TSR, an anti-PS antibody, and derivatives thereof. In some embodiments, the reagent that selectively and/or reversibly binds PS on the sperm cells is detectably labeled. In some embodiments, isolating sperm bound to the reagent that that selectively and/or reversibly binds PS comprises employing flow cytometry (FCM) or fluorescence-activated cell sorting (FACS). Any suitable FCM or FACS technique as would be apparent to one of ordinary skill in the art upon a review of the instant disclosure is provided in accordance with the presently disclosed subject matter. By way of example and not limitation, sperm cells can be labeled with Annexin V as described herein and FACS performed.

In some embodiments, the reagent that selectively and/or reversibly binds PS further comprises a capture moiety and isolating the sperm cells comprises isolating the capture moiety. In some embodiments, the capture moiety comprises a substrate. The reagent that binds PS can be reversible. Thus, in some embodiments, the reagent that selectively and/or reversibly binds PS is bound to the substrate. In some embodiments, the substrate comprises a microbead or a nanobead. In some embodiments, the method further comprises eluting the sperm cells from the reagent that selectively and/or reversibly binds PS on the sperm cells. By way of example and not limitation, a spermatozoa-microbeads suspension is loaded on a column containing a coated cell-friendly matrix containing iron balls, which was fitted in a magnet (MiniMACS; Miltenyi Biotec). The power of magnetic field is measured as about 0.5 Tesla between the poles and up to about 1.5 Tesla within the iron globes of the column. The fraction containing the PS+ sperm (due to binding to Annexin V beads) is retained in the column, and the then sperm will be eluted with a Ca⁺⁺ free buffer. As the binding of Annexin V to PS is Ca⁺⁺ dependent, the Ca⁺⁺ free buffer releases the PS+ sperm from the Annexin microbeads. See Example 8 and FIG. 16.

In some embodiments, the method further comprises using the results of the sperm cell analysis to determine an assisted reproductive technology (ART) treatment pathway (e.g., intra-uterine insemination (IUI), in vitro fertilization (IVF), or intra-cytoplasmic sperm injection (ICSI)) for a subject. In some embodiments, the instruction material includes instructions for determining an assisted reproductive technology (ART) treatment pathway (e.g., intra-uterine insemination (IUI), in vitro fertilization (IVF), or intra-cytoplasmic sperm injection (ICSI)) for a subject. Thus, the presently disclosed subject matter provides tools to assess sperm fertility to improve the quality of information that millions of couples currently lack when determining their ART treatments. Moreover, male fertility is a barometer of men's health with clear associations to diabetes, heart disease, and mortality. Thus, in some embodiments, the method further comprises discussing and/or suggesting additional health care interventions to a male patient.

### III.C. METHODS AND KITS FOR ASSESSING FUSION COMPETENCY SPERM CELLS

In some embodiments, the presently disclosed subject matter provides methods of assessing fusion competency in sperm cells. In some embodiments, the method comprises (a) providing sperm cells from a subject; (b) labeling sperm with a reagent that can be subsequently tracked to assess fusion (such as calcein or DiI dyes, or other examples disclosed elsewhere herein); (c) adding the sperm cells to a culture comprising myoblasts (e.g. C2C12 mouse myoblast line or primary myoblasts), or other fusion competent cells (e.g. trophoblast cells or osteoclast cells); and (d) assessing fusion competency of the sperm cells by detecting the label transfer to myoblasts (or similar fusion-competent cells that can used as in vitro surrogates for oocytes) in the culture. In some embodiments, the fusion-competent cell culture comprises any suitable number of cells, such as but not limited to about 10,000 to about 50,000 cells.

In some embodiments, the subject is a human subject or a non-human animal subject. In some embodiments, the non-human animal subject is selected from the group consisting of bovine, equine, porcine, ovine, canine, feline, and avian.

In some embodiments, the sample is formed by concentrating a semen sample from the subject. Approaches, reagents, and/or devices for concentrating a semen sample are disclosed elsewhere herein and include, but are not limited to, approaches, reagents, and/or devices enriching sperm in the sample using an isolation gradient. Indeed, any suitable reagent, device, and/or approach for concentrating sperm in a sample as would be apparent to one of ordinary skill in the art upon a review of the instant disclosure is provided in accordance with the presently disclosed subject matter.

In some embodiments, the method further comprises adding a contrast agent to the culture. In some embodiments, the contrast agent comprises a Hoechst counterstaining agent.

In some embodiments, a kit for assessing fusion competency in sperm cells is provided. In some embodiments, the kit comprises a reagent that can be subsequently tracked to assess fusion (such as calcein or DiI dyes, or other examples disclosed elsewhere herein); a culture comprising myoblasts (e.g. C2C12 mouse myoblast line or primary myoblasts), or other fusion competent cells (e.g. trophoblast or osteoclast cells); and instructional material for assessing fusion competency in the sperm cells by detecting the reagent in the culture. In some embodiments, the fusion-competent cell culture comprises any suitable number of cells, such as but not limited to about 10,000 to about 50,000 cells.

In some embodiments, the kit comprises a reagent for concentrating sperm cells in a sample. Approaches, reagents, and/or devices for concentrating a semen sample are disclosed elsewhere herein and include, but are not limited to, approaches, reagents, and/or devices enriching sperm in the sample using an isolation gradient. Indeed, any suitable reagent, device, and/or approach for concentrating sperm in a sample as would be apparent to one of ordinary skill in the art upon a review of the instant disclosure is provided in accordance with the presently disclosed subject matter.

In some embodiment, the kit further comprises a contrast agent for the culture. In some embodiments, the contrast agent comprises a Hoechst counterstaining agent.

In some embodiments, the method further comprising using the results of the sperm cell analysis to determine the assisted reproductive technology (ART) treatment pathway (e.g., intra-uterine insemination (IUI), in vitro fertilization (IVF), or intra-cytoplasmic sperm injection (ICSI)) for a subject. Thus, the presently disclosed subject matter provides tools to assess sperm fertility to improve the quality of information that millions of couples currently lack when determining their ART treatments. Moreover, male fertility is a barometer of men's health with clear associations to diabetes, heart disease, and mortality. Thus, in some embodiments, the method further comprises discussing and/or suggesting additional health care interventions to a male patient.

### III.D. CONTRACEPTIVE METHODS, COMPOSITIONS AND DEVICES

The presently disclosed subject matter provides new insights into the fusion mechanism between the sperm and the oocyte. In some aspects the presently disclosed subject matter shows that fusion, and thus fertilization, depends on the presence of phosphatidylserine (PS) on sperm, a lipid whose exposure has been studied in cells undergoing apoptosis. PS is prominently exposed on the head region of viable and motile murine sperm. Masking the PS on sperm via different approaches prior to mixing with oocytes strongly inhibits fertilization without affecting sperm motility. Complementarily, oocytes express PS receptors and fertilization was affected by targeting the oocyte PS receptors. Further, oocytes from mice lacking ELMO1 (which functions downstream of the PS receptor BAI1), or interference with RAC1 in oocytes (which is activated downstream of BAI1/3 and ELMO), also affected sperm entry into oocytes.

In some embodiments, the presently disclosed subject matter provides contraceptive methods. In some embodiments, a method of blocking fertilization is provided. In some embodiments, the method comprises providing a reagent that blocks PS binding between a sperm cell and an oocyte, and administering a composition comprising the reagent topically to a subject at a time of sexual intercourse. In some embodiments, the administering comprising applying the composition comprising the reagent to a contraceptive device. In some embodiments, the reagent selectively binds PS. In some embodiments, the reagent that blocks PS binding between a sperm cell and an oocyte blocks one or more individual PtdSer receptors on the oocyte, such as via antibody-mediated blocking. In some embodiments, the reagent that blocks PS binding between a sperm cell and an oocyte is selected from the group consisting of Annexin and GST-BAI-TSR, and derivatives thereof.

In some embodiments, the reagent that blocks binding of PS on the sperm cell with PS receptors on the oocyte comprises an oocyte PS receptor blocking agent. In some embodiments, the oocyte PS receptor blocking agent comprises (a) an antibody against a PS receptor selected from the group consisting of CD36, BAI1, BAI3, Tim4, and Mer-TK; (b) any combination of one or more antibodies against a PS receptor selected from the group consisting of CD36, BAI1, BAI3, Tim4, and Mer-TK, and (c) a derivative of an antibody of (a) or (b). In some embodiments, the oocyte PS receptor blocking agent comprises a modified bead, such as a carboxylate modified bead.

In some embodiments, the presently disclosed subject matter provides a contraceptive device comprising a coating or lubricant comprising a reagent that blocks PS binding between a sperm cell and an oocyte. In some embodiments, the reagent that blocks PS binding between a sperm cell and an oocyte is chosen from the group consisting of Annexin V, GST-BAI1-TSR and derivatives thereof. Representative contraceptive devices include but are not limited to a diaphragm, male condom, female condom, or other device as would be apparent to one of ordinary skill in the art upon a review of the instant disclosure.

In some embodiments, the reagent that blocks binding of PS on the sperm cell with PS receptors on the oocyte comprises an oocyte PS receptor blocking agent. In some embodiments, the oocyte PS receptor blocking agent comprises (a) an antibody against a PS receptor selected from the group consisting of CD36, BAI1, BAI3, Tim4, and Mer-TK; (b) any combination of one or more antibodies against a PS receptor selected from the group consisting of CD36, BAI1, BAI3, Tim4, and Mer-TK, and (c) a derivative of an antibody of (a) or (b). In some embodiments, the oocyte PS receptor blocking agent comprises a modified bead, such as a carboxylate modified bead.

In some embodiments, the presently disclosed subject matter provides a composition comprising a reagent that blocks PS binding between a sperm cell and an oocyte; and a pharmaceutically acceptable carrier. In some embodiments, the reagent that blocks PS binding between a sperm cell and an oocyte is selected from the group consisting of Annexin V, GST-BAI1-TSR, and derivatives thereof.

In some embodiments, the reagent that blocks binding of PS on the sperm cell with PS receptors on the oocyte comprises an oocyte PS receptor blocking agent. In some embodiments, the oocyte PS receptor blocking agent comprises (a) an antibody against a PS receptor selected from the group consisting of CD36, BAI1, BAI3, Tim4, and Mer-TK; (b) any combination of one or more antibodies against a PS receptor selected from the group consisting of CD36, BAI1, BAI3, Tim4, and Mer-TK, and (c) a derivative of an antibody of (a) or (b). In some embodiments, the oocyte PS receptor blocking agent comprises a modified bead, such as a carboxylate modified bead.

Thus, the disclosed compositions can be employed by administration to a subject in need thereof. In some embodiments, the disclosed pharmaceutical compositions can be administered in vivo in a pharmaceutically acceptable carrier. By “pharmaceutically acceptable” is meant a material that is not biologically or otherwise undesirable, i.e., the material can be administered to a subject, along with a peptide composition of the presently disclosed subject matter, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art. The materials can be in solution and/or in suspension. Suitable carriers and their formulations are described in Remington et al. (1975) Remington's Pharmaceutical Sciences. 15th ed., Mack Pub. Co., Easton, Pa., United States of America. It will be apparent to those persons skilled in the art that certain carriers can be selected depending upon, for instance, the route of administration and/or concentration of composition being administered.

Pharmaceutical compositions can include carriers, thickeners, diluents, buffers, preservatives, surface active agents, and the like, in addition to the molecule of choice. Pharmaceutical compositions can also include one or more active ingredients such as antimicrobial agents, anti-inflammatory agents, and the like.

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

### Materials and Methods for Examples 1-4

Mice. C57BL/6 mice (stock 000664) and Ddx4-Cre mice (stock 006974) were purchased from the Jackson Laboratory (Bar Harbor, Me., United States of America) and bred in our facilities. BAI1 deficient and Elmo1^{fl/fl} mice were previously generated in our laboratory¹¹. Mer-tk deficient mice (stock 011122) were purchased from the Jackson Laboratory and crossed with BAI1 deficient mice in our facilities. Tim-4 deficient mice were kindly provided by Dr. Vijay Kuchroo (Brigham and Women's Hospital, Massachusetts, United States of America). Yellow fluorescent protein (YFP) expressing mice (stock 006148) were crossed to E2A-Cre mice (stock 003724), both from the Jackson Laboratory. All animal procedures were approved by and performed according to the guidelines of the Institutional Animal Care and Use Committee (IACUC) at the University of Virginia, Charlottesville, Va., United States of America.

Sperm staining. To stain sperm with Annexin V, the caput, corpus and cauda epididymis of adult (>10 week-old) male mice were dissected and the sperm were allowed to disperse for 15 min in capacitating medium [TYH+BSA [119 mM NaCl, 4.7 mM KCl, 1.71 mM CaCl₂), 1.2 mM KH₂PO₄, 25.1 mM NaHCO₃, 5.56 mM glucose, 0.51 mM Na pyruvate, 1% phenol red, supplemented with 4 mg/ml BSA, penicillin and streptomycin]⁶⁶]. For capacitation studies, caudal sperm were allowed to swim in capacitating medium (described elsewhere herein) or non-capacitating medium (lacking CaCl₂), NaHCO₃ (replaced with HEPES) and 4 mg/ml BSA) and incubated in the respective medium for additional 90 min. Sperm were counted and 1×10⁶ cells were washed in 1× binding buffer and stained with Alexa-Fluor 568 conjugated Annexin V (Life Technologies, Waltham, Mass., United States of America) for 15 min. After washing with binding buffer and staining with Hoechst, sperm were placed on slides and 6-8 photographs were taken per sample. The percentage of Annexin V+ sperm (stained on head, midpiece or midpiece and head) was calculated.

To visualize the motility of Annexin V⁺ sperm, caudal sperm were obtained as described above, capacitated for 90 min in TYH+BSA at 37° C., 5% CO₂ and stained with Annexin V conjugated with Alexa-Fluor 488 (Life Technologies) for 15 min at room temperature. Sperm aliquots were placed on 20 μm chamber slides (Leja) and analyzed in a Ziess LSM 880 confocal microscope (Microscope Facility Core, University of Virginia, Charlottesville, Va., United States of America). To detect PtdSer, sperm was also incubated with BAI1-TSR-GST or GST only (50 μg/ml), washed and fixed with 2% paraformaldehyde and placed on slides. GST was detected with a specific antibody (GST Ab, Santa Cruz, sc-138B) followed by a biotinylated secondary antibody and NA-Texas Red.

To test the staining sperm with Duramycin, sperm from the cauda epididymides were obtained as described above, incubated with Duramycin-LC-biotin conjugate (Molecular Targeting Technologies), washed and stained with NA-Texas Red (Southern Biotech). After an additional wash, sperm were stained with Hoechst and mounted in coverslips.

To stain sperm with the dyes Calcein-AM or DiI, caudal sperm were allowed to swim in TYH+0.75 mM Methyl-beta-cyclodextrin (MBCD)⁶⁷ for 15 min, counted and stained with 4 μM Calcein red orange-AM (Invitrogen) for 30 min in PBS at room temperature or with 5 μg/ml DiI (Invitrogen) for 30 min at 37° C. in HBSS, as previously described⁶⁰. Cells were washed, counted and resuspended in DMEM medium supplemented with 20% heat inactivated FBS.

In vitro fertilization (IVF) assays. IVF was performed as previously described⁶⁸. Briefly, 4-5 week old female mice were super-ovulated by intraperitoneal injections of 5 IU of pregnant mare serum gonadotropin (PMSG, Prospec) and 5 IU of human chorionic gonadotropin (hCG, Sigma) 48 hours (h) later. Metaphase II oocytes were recovered 13 h after hCG, and were: 1) directly used as cumulus oocyte complexes (COC), 2) freed from cumulus cells by a brief incubation with 3 mg/ml hyaluronidase (Sigma) in 0.01% poly vinyl alcohol (PVA)/FHM medium (EMD Millipore). These oocytes were labeled as Zona pellucida (ZP)-intact oocytes; or 3) freed from cumulus cells and ZP using Tyrode's solution (Sigma) (ZP-free oocytes). Sperm were collected from the cauda epididymides of adult (>10 week-old) wild-type male mice and capacitated for 90 min in TYH+BSA medium (described above, Sperm staining section). All the incubations were performed in medium drops under paraffin oil (Sigma) at 37° C., 5% CO₂ atmosphere.

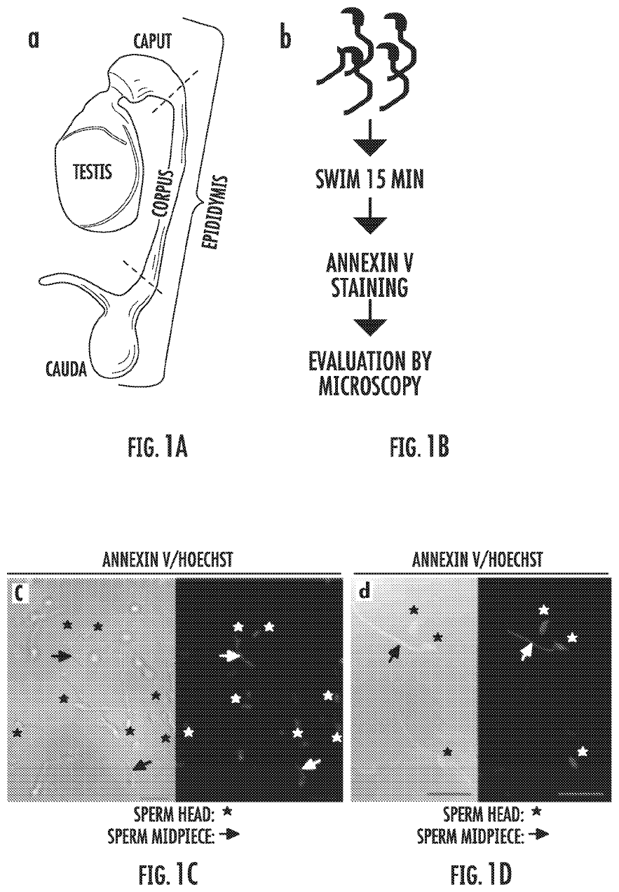

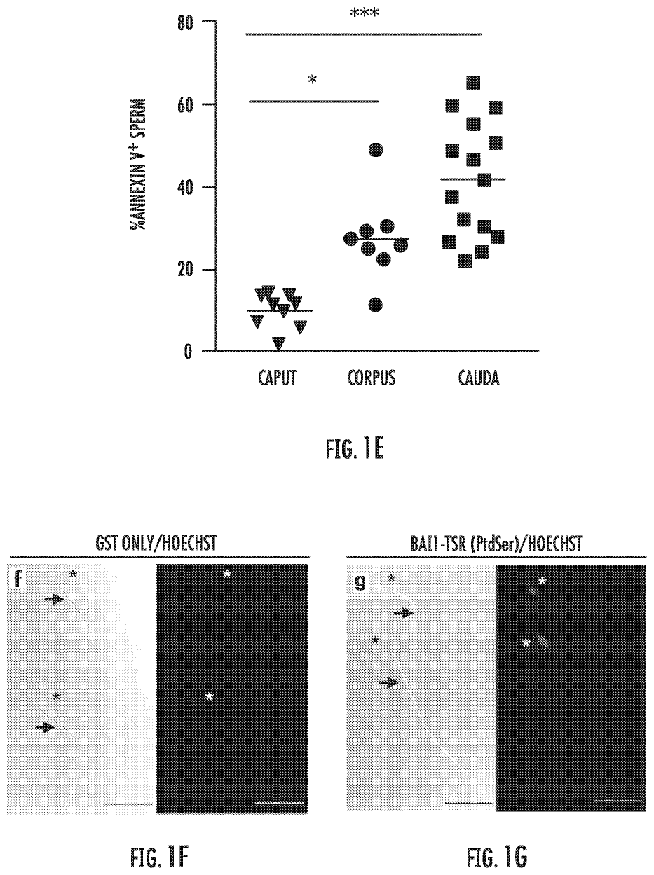

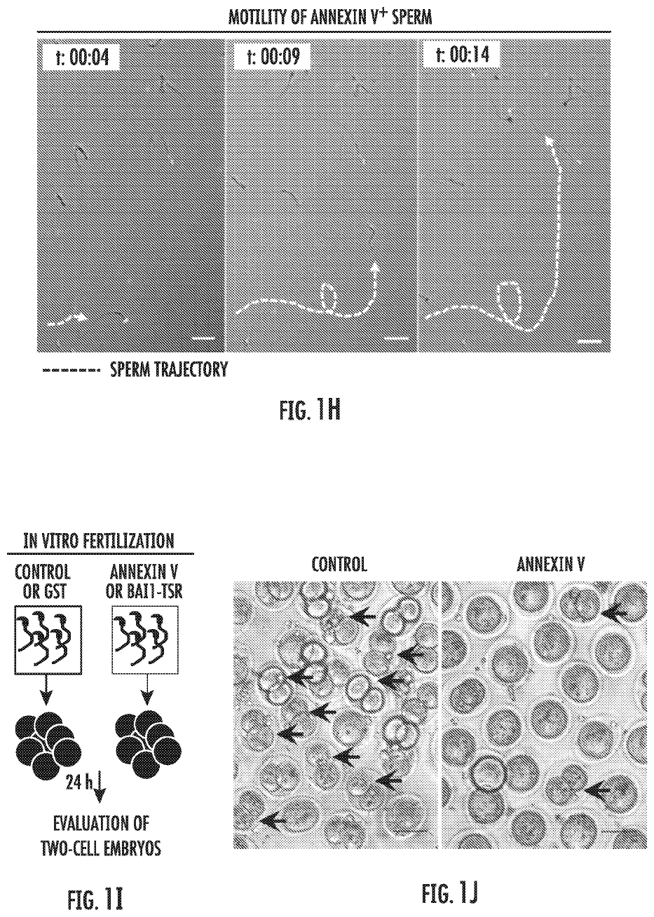

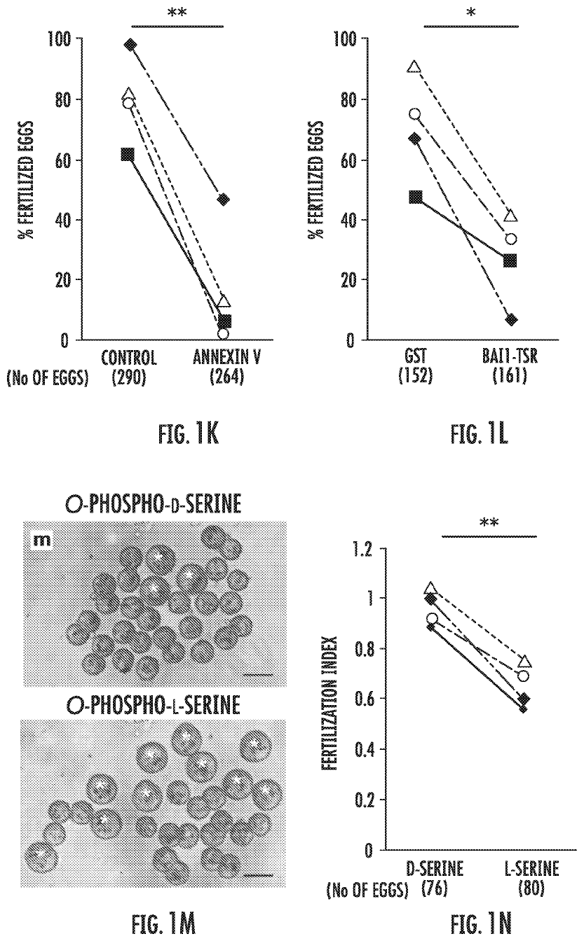

To evaluate the role of PtdSer exposed on sperm, cumulus oocyte complexes (n=30-60) were inseminated with 2-4×10⁴ sperm capacitated for 60-90 min in TYH+BSA (in most of the experiments) or in TYH+MBCD⁶⁷ (in the experiment to test the effect of Annexin V in fertilization, FIG. 1K). During the last 30 min of capacitation, sperm were incubated with 10 μg/ml Annexin V (Ebiosciences), 50 μg/ml GST or 50 μg/ml GST-BAI1-TSR. After co-culture of oocytes and sperm in 100 μl drops of high calcium human tubal fluid (HTF) supplemented with 1 mM reduced glutathione (GSH) medium for 4 hours⁶⁷, eggs were washed several times and transferred to KSOM medium (EMD Millipore). The percentage of 2-cell embryos (fertilized eggs) was determined at 24 hours post insemination. Sperm motility upon incubation with control medium, Annexin V, GST or GST-BAI1-TSR was graded as progressive motility, non-progressive motility, and immotility, according to the criteria of the 5^{th} Edition of the World Health Organization (WHO) Laboratory Manual for the Examination and Processing of Human Semen. The non-progressive motility and immotility numbers were grouped together. To test the soluble head group of PtdSer, O-Phospho-L-Serine (Sigma) and its control O-Phospho-D-Serine (Sigma) in IVF assays, ˜20 ZP-free oocytes isolated from wt mice were incubated 1×10³ capacitated sperm in 20-μl droplets for 1 h in the presence of 1 mM O-Phospho-L-Serine or O-Phospho-D-Serine. After washes and an overnight incubation the percentage of 2-cell embryos was evaluated.

To genetically test the role of PtdSer recognition receptors in the fertilization assays, BAI1−/−, ELMO1−/−, Tim-4−/−, Mer-tk−/−, Mer-tk−/− BAI1−/− and wild-type female mice were super-ovulated as described above and cumulus oocytes complexes were inseminated with 2×10⁵ capacitated sperm in 100 μl drops of TYH+BSA for 4 hours. After several washes, oocytes were transferred to KSOM medium and the percentage of 2-cell embryos was evaluated at 24 h post insemination.

To evaluate the role of BAI1/3 and CD36 using blocking antibodies (Abs), we incubated cumulus free ZP-intact oocytes with BAI1/3 (50 μg/ml, R&D Systems, AF4969), CD36 (10 μg/ml, clone CFR D-2712, Hycult, HM1074), Juno (10 μg/ml, clone TH6, Biolegend, 125102), CD9 (50 μg/ml, clone KMC8, BD Biosciences, 553758) antibodies or isotype controls: mouse IgA, (10 μg/ml, Southern Biotechnologies, 0106-14) or sheep IgG, (50 μg/ml, R&D systems, 5-001-A) in TYH+BSA+5% FBS (to avoid ZP hardening) for 1 h at 37° C., 5% CO₂. After several washes in TYH+BSA (to eliminate the FBS) 25-30 oocytes were inseminated with 4×10⁴ capacitated sperm in 100 μl TYH+BSA in the presence or absence of specific antibodies or isotype controls for 3 h. Oocytes were washed in TYH+BSA and transferred to KSOM medium for an overnight incubation at 37° C., 5% CO₂. The fertilization index was calculated as the percentage of fertilized eggs (2-cell embryos) in the experimental group divided the percentage of fertilized eggs observed in the control group.

To score fertilization via the sperm DNA decondensation assay, we incubated cumulus free ZP-intact oocytes with CD36, BAI1/3 or the isotype controls as described above, and then loaded the oocytes with 10 μg/ml DAPI (BioRad) for 20 min at 37° C., 5% CO₂, and washed several times in TYH+BSA³. Oocytes were inseminated for 3 h, washed and fixed with 0.25% glutaraldehyde and 0.1% paraformaldehyde for 20 min at room temperature. The percentage of oocytes with DAPI⁺ decondensed sperm nuclei (typically 1-2/oocyte) was evaluated by microscopy.

To evaluate the role of RAC1, ZP-free oocytes were prepared as described above, loaded with 10 μg/ml DAPI (BioRad) for 20 min at 37° C., 5% CO₂, and washed several times in TYH+BSA 3. Twenty oocytes, either untreated or previously incubated with RAC1 inhibitor (80 μM, EHT-1864, Sigma) for 1-2 h were inseminated with 1000 capacitated sperm for 1 h in 20 μl drops of TYH+BSA medium at 37° C., 5% CO₂. RAC1 inhibitor was also present the fertilization steps. After 3 washes, oocytes were fixed in 0.25% glutaraldehyde and 0.1% paraformaldehyde for 20 min at room temperature, washed and mounted. The percentage of oocytes with DAPI⁺ decondensed sperm nuclei (typically 1-2/oocyte) was evaluated by microscopy.

Staining of mouse/human eggs with antibodies. ZP-free oocytes were obtained from wild-type female mice after removal of cumulus cells and the ZP via treatment with hyaluronidase and Tyrode's solution, respectively. Live oocytes were incubated with antibodies to BAI1/3 (R&D Systems), CD36 (Hycult), CD9 (BD Pharmingen, 553758), Juno (Biolegend, 125102), or both CD36+BAI1/3 Abs in TYH+BSA drops under paraffin oil at 37° C., 5% CO₂ for 1 h. Antibodies concentrations were the same as described above (IVF section). For the staining of microvilli, oocytes were incubated with 50 μg/ml FITC-Concanavalin A³⁷ (Vector, FL-1001) for 5 min. Cells were fixed in 4% PFA+1% BSA for 30 min at room temperature, washed and incubated with biotinylated secondary or Alexa-Fluor 488 secondary antibodies for 1 h at room temperature. After washing, cells were stained with NA-Texas Red and Hoechst in 1% BSA. For ELMO1 staining, oocytes were fixed in 4% PFA+1% BSA and permeabilized with 0.5% triton X-100+5% BSA and the stained with an ELMO1 antibody (Abcam, ab2239) overnight at 4° C. ZP-intact human oocytes were stained with BAI1/3 or CD36 antibodies and fixed as described with mouse oocytes. Informed consent was obtained from female IVF patients undergoing treatment at the Reproductive Medicine and Surgery Center of Virginia to use discarded, de-identified unfertilized human oocytes following approval by the Sentara Martha Jefferson Hospital Institutional Review Board (IRB #06-017).

The presence of functional PtdSer receptors on mouse ZP-free wild-type oocytes was evaluated by incubation with 2 μm carboxylate modified red (580/605) beads (1:400, Invitrogen, FluoSpheres) for 2 h in TYH+BSA at 37° C., 5% CO₂. Beads were pre-treated with medium only, 50 μg/ml GST or 50 μg/ml GST-BAI1-TSR for 30 min. Oocytes were washed 6 times in TYH+BSA, stained with CD9 antibody for 30 min at 37° C., 5% CO₂ in TYH+BSA, fixed in 4% PFA+1% BSA and stained with Hoechst. The percentage of oocytes with bound beads was determined by microscopy. We scored oocytes with >5 bound beads, since accurately counting individual beads, particularly in oocytes of the control groups that bound multiple beads was unfeasible.

Immunostaining of ovarian tissue. Mouse ovaries obtained from 4-6 week-old wild-type female mice were fixed in Bouin's solution for 6 h and embedded in paraffin (Histology Core, University of Virginia). Sections were deparaffinized and hydrated, and stained with BAI1/3, Connexin-43 (Abcam, ab11370) or ELMO1 antibodies as described above for isolated oocytes. Human ovary was stained with BAI1/3 Ab (R&D Systems) by the Biorepository and Tissue Research Facility at the University of Virginia.

Sperm:myoblast fusion experiments. C2C12 murine skeletal muscle myoblasts (ATCC) were maintained at sub-confluent densities in DMEM medium supplemented with 20% heat inactivated FBS at 37° C., 8.5% CO₂. Cells were trypsinized, counted and plated in 4-well LabTek II Permanox chamber slides (25,000 cells/well). After 24 h, myoblasts were treated with appropriate buffer alone, or 5 μg/ml Cytochalasin D (Sigma), 80 μM RAC1 inhibitor (EHT-1864), or incubated with Abs (BAI3 [R&D Systems, MAB39651] or CD9 [BD Biosciences]) for 30 min at 37° C., 8.5% CO₂ prior to the sperm co-incubation. Alternatively, myoblasts were fixed with 4% PFA for 20 min and washed several times. Calcein-AM+ sperm (250,000 sperm/well) were added and incubated for 4 h at 37° C., 8.5% CO₂. The blocking agents were also present during the co-incubation except for Cytochalasin D, which was used only during the preincubation to avoid affecting the sperm. Sperm were pretreated with 50 μg/ml GST or 50 μg/ml GST-BAI1-TSR for 30 min and then added to the myoblasts. C2C12 myoblasts with shRNA-mediated Elmo2 knockdown have been described previously²⁵. After the 4 h co-incubation, myoblasts were washed, stained with Hoechst and mounted. Cells were analyzed the same day and 6-8 fields/well were photographed using the Axio Imager 2 with Apotome (Zeiss). Image J cell counter plugin software was used to quantitate nuclei number. The fusion index was calculated as the percentage of Calcein AM⁺ myoblasts in the experimental group normalized to the control untreated cells.

Primary myoblasts were isolated from 3-6 week-old mice as previously described^{69, 70} and plated in Matrigel-coated dishes. Detection of BAI1/3, CD36 and CD9 was performed by immunofluorescence as described above. For the detection of YFP sperm within myoblasts, YFP+ sperm was co-cultured with C2C12 myoblasts for 4 h as described above, and fixed in 3.7% formaldehyde for 5 min at room temperature. Cells were permeabilized with 0.5 Triton X-100 for 10 min and incubated overnight at 4° C. with antibodies to GFP/YFP (Abcam, ab6673) and Izumol (ProSci, 8233) diluted in 0.1% Tween-20 and 0.5% BSA. Secondary antibodies conjugated with Alexa Fluor 647 or biotin were used. Streptavidin-Texas Red, Phalloidin (Invitrogen) and Hoechst were also used. Myoblasts were analyzed with a LSM880 confocal microscope. The YFP/GFP antibody staining was necessary as the endogenous fluorescence of YFP was too weak to detect after fixation. For electron microscopy, unlabeled sperm were co-cultured with C2C12 myoblasts for 4 h. Cells were washed to remove the residual unfused sperm, trypsinized, and washed again before fixation with 2.5% glutaraldehyde and 2% paraformaldehyde, and processing for analysis via electron microscopy.

Quantitative RT-PCR. Total RNA was extracted from cumulus free ZP-intact oocytes from wild-type female mice using Trizol (Ambion) and the cDNA was synthesized using QuantiTect Reverse Transcription Kit (Qiagen) according to manufacturer's instructions. qPCR for mouse Bai1, Bai2, Bai3, Tim4, Stab2, Elmo1, Elmo2, cd36, cd52, Juno or housekeeping gene Gapdh was performed using Taqman probes (Applied Biosystems) using StepOnePlus Real Time PCR System (ABI). Cd52 was used to determine oocyte contamination with cumulus cells.

Statistical analysis. Statistical significance was determined using GraphPad Prism 5 or 6 using unpaired Student's two-tailed t-test, one-sample t-test, Mann-Whitney test, or one-way ANOVA as according to test requirements. No inclusion/exclusion criteria were pre-established. A p-value of <0.05 (*), <0.01 (**), or <0.001 (***) were considered significant.

### Example 1

### Phosphytidyl Serine (PtdSer) Exposed on Viable Sperm is Required for Fertilization

Annexin V staining was observed on freshly isolated sperm from the cauda epididymis (FIGS. 1A, 1B) suggestive of PtdSer exposure (FIGS. 1C, 1D). Although PtdSer has been noted on sperm previously¹²⁻¹⁴, it was considered to mark dead or non-viable sperm because PtdSer is a key eat-me signal on cells undergoing apoptosis, that facilitates recognition and uptake by phagocytes¹⁵. The relevance of PtdSer exposure on the sperm was further investigated.

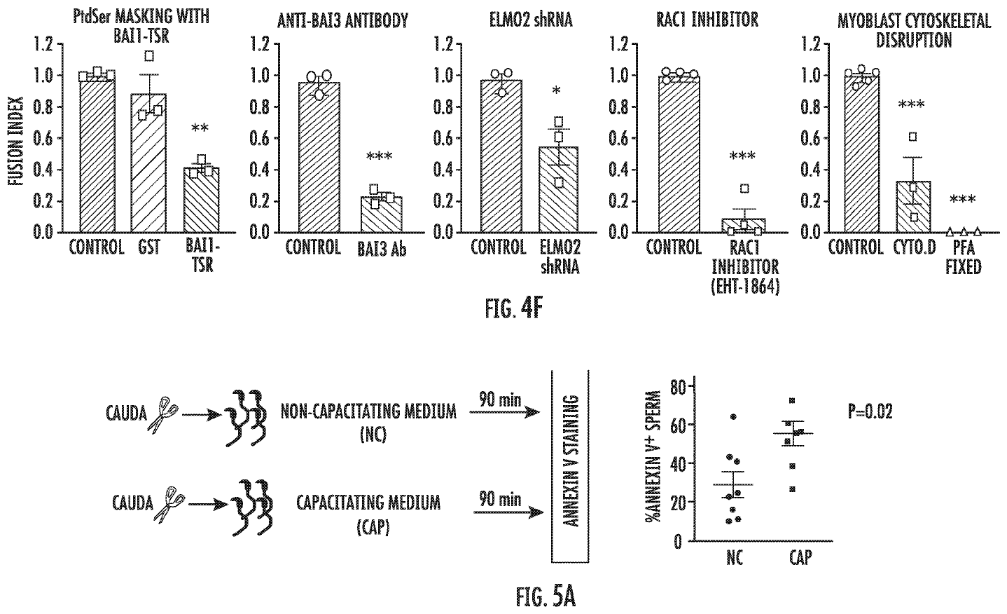

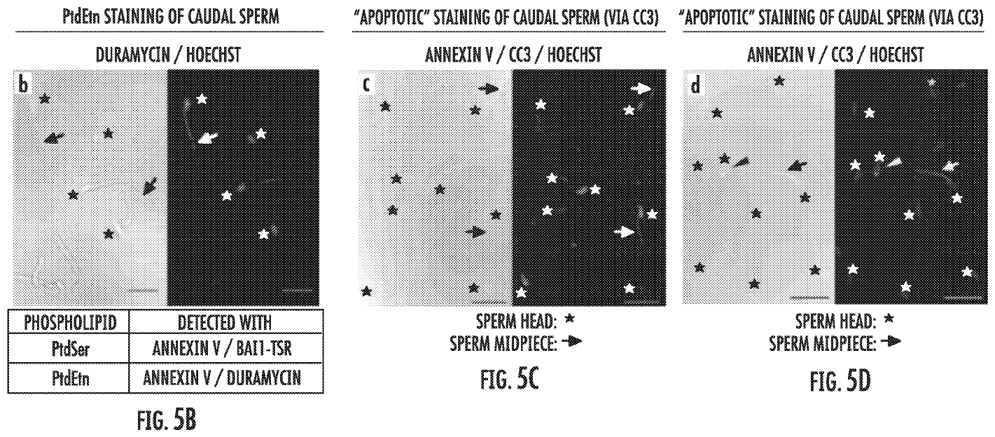

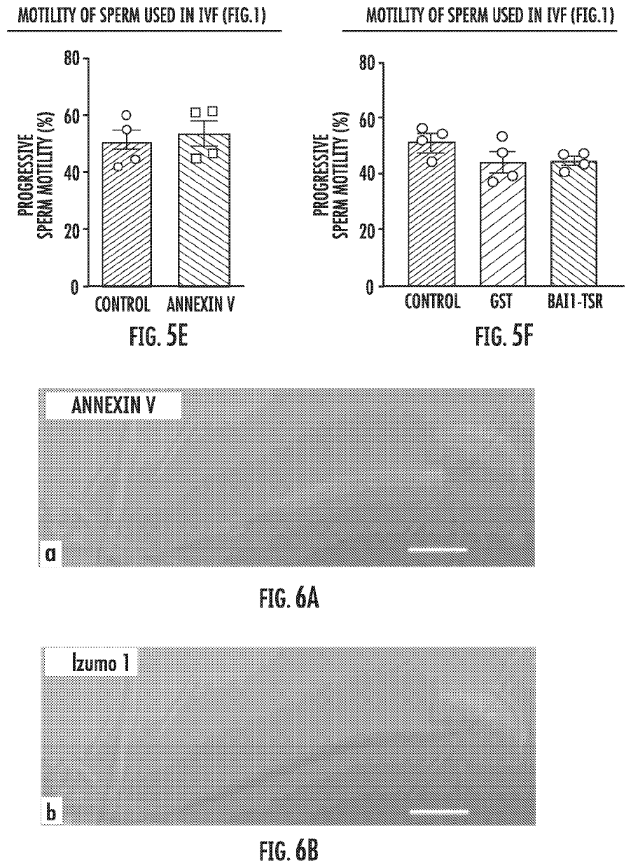

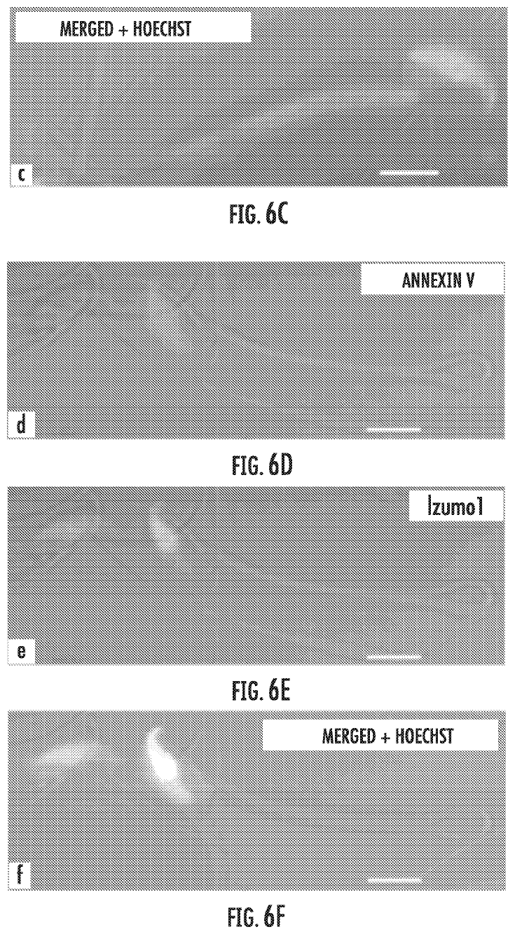

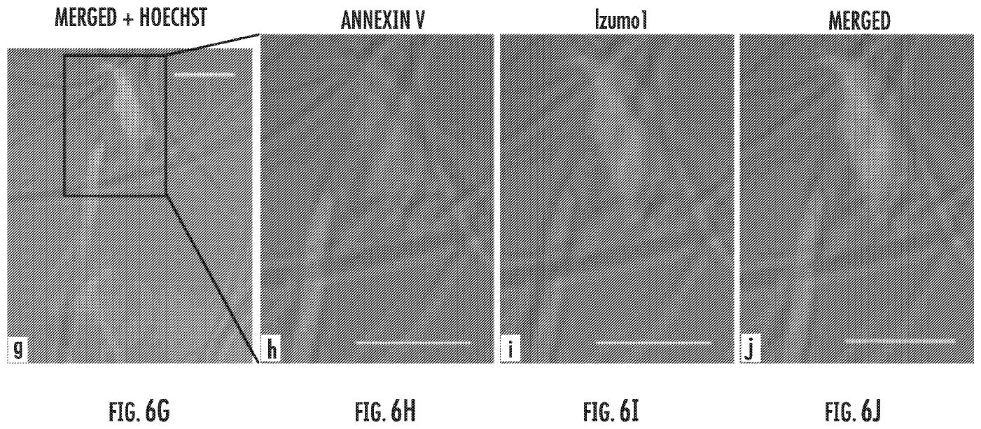

Annexin V staining was prominently seen both on the sperm head and the midpiece, but was absent in the tail (FIGS. 1C, 1D). During spermatogenesis, after exiting the testis, sperm transits through different segments of the epididymis: the caput, the corpus, and the cauda (FIG. 1A). Classical experiments have shown that only the caudal sperm is capable of fertilization²⁰. Therefore, PtdSer exposure on sperm was assessed as it transits through the epididymis. A progressive increase in PtdSer exposure on sperm isolated from different segments of the epididymis was observed, with the cauda epididymis, which contains the fertilization-competent sperm, displaying the highest percentage of PtdSer-positive sperm (FIG. 1E). This also indicates that PtdSer externalization is not merely an effect of sperm isolation. When we addressed whether PtdSer exposure on sperm changes with capacitation, a process known to occur in the female reproductive tract²¹, we found a further increase in the percentage of PtdSer-positive sperm after capacitation in vitro (FIG. 5A). The acrosome reaction is another process that occurs on sperm in the female tract. When we induced the acrosome reaction in vitro with the ionophore A23187, PtdSer continued to be detectable on sperm. Further, as Izumol is exposed on caudal sperm after the acrosome reaction and is a central player in fertilization⁴, we asked whether PtdSer colocalizes with Izumol on sperm. Izumol (detected via antibody), as well as PtdSer (via Annexin V), were colocalized in the equatorial region of the sperm head (known to be involved in sperm:oocyte fusion²) (FIG. 6). Since Annexin V can bind both PtdSer and phosphatidylethanolamine (PtdEtn)²², we tested PtdSer exposure on the sperm using a second reagent. We have previously established that the soluble extracellular fragment of the PtdSer recognition receptor BAI1 (BAI1-TSR fused to the glutathione-S-transferase [GST]) binds PtdSer but not PtdEtn²³; BAI1-TSR (but not the control GST protein) preferentially bound to the sperm head without significant midpiece binding (FIGS. 1F, 1G). Consistent with this observation, when we used Duramycin, which binds PtdEtn but not PtdSer 2, the binding was noted prominently in the midpiece with much less binding to the head (FIG. 5B). These data suggested specific exposure of PtdSer on the heads of freshly isolated sperm from the cauda epididymis.

To address whether the sperm with PtdSer exposed on their surface are viable, we performed time-lapse microscopy, revealing that the PtdSer⁺ sperm were motile (FIG. 1H). Further, when we analyzed the expression on the caudal sperm of cleaved caspase 3 (CC3), a marker for apoptosis, the Annexin V⁺ sperm were negative for CC3 (FIG. 5C). Only after 24 h incubation ex vivo, some of the sperm showed CC3 staining (FIG. 5D). These data suggested that PtdSer is exposed on the surface of viable and motile sperm.

We next asked whether the PtdSer exposure on sperm is functionally important for fertilization. To test this, we performed in vitro fertilization using capacitated caudal sperm and oocytes from super-ovulated C57BL/6 female mice, and quantified the emergence of 2-cell embryos (see schematic in FIG. 1I). We deliberately chose three different reagents to target PtdSer, as each have unique features that are complementary, and can inform us better about the role of PtdSer during fertilization: 1) Annexin V (which has the highest affinity); 2) BAI1-TSR peptide derived from the extracellular region of the PtdSer receptor BAI1 fused to the GST (lower affinity than Annexin V but greater specificity for PtdSer); and 3) the soluble head group of the lipid phosphatidylserine, which has the lowest affinity of the three blocking agents but can act as a competitive inhibitor for PtdSer recognition receptors. Pleasingly, all three reagents supported the hypothesis that PtdSer on sperm contributes to fertilization. Annexin V caused >85% reduction in fertilization in three out of four independent experiments (FIGS. 1J, 1K). Masking PtdSer on sperm with BAI1-TSR also significantly reduced fertilization, and consistent with the BAI1-TSR being of lower affinity than annexin V, the inhibition of fertilization with BAI1-TSR was less pronounced (FIG. 1L). Addition of the soluble Phospho-L-Serine head group also resulted in significant reduction in fertilization in every experiment (FIGS. 1M, 1N). The partial inhibition (30-40%) was expected as the monomeric soluble head groups of PtdSer have to compete against multi-valent PtdSer recognition on the sperm surface. The advantage of using the Phospho-L-serine blocking is that we could directly compare its effect against the stereoisomer Phospho-D-Serine as a control (carrying the same charge), and this did not inhibit fertilization. Of note, we confirmed that progressive motility of the sperm was not affected after masking PtdSer with Annexin V or BAI1-TSR (FIGS. 5E, 5F). Collectively, these three approaches suggest that recognition of PtdSer on sperm (in addition to the well-described Izumol) can contribute to in vitro fertilization.

### Example 2

### PtdSer Receptors on Oocytes Contribute to Fertilization

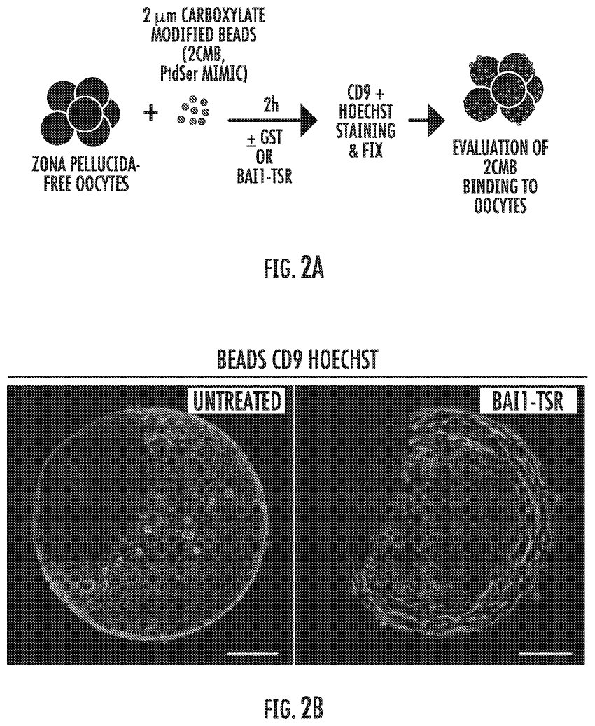

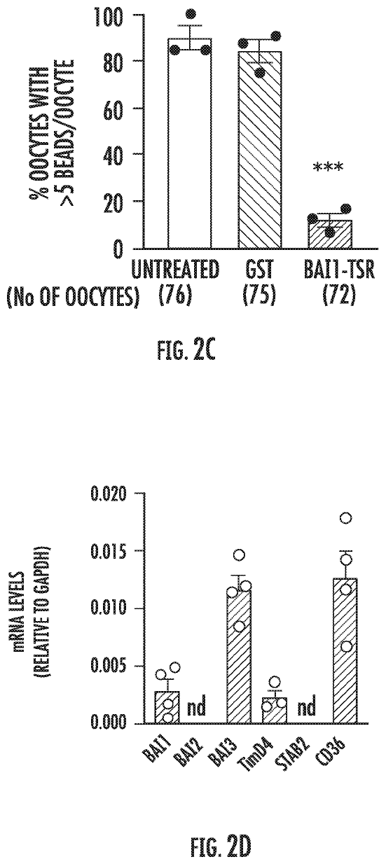

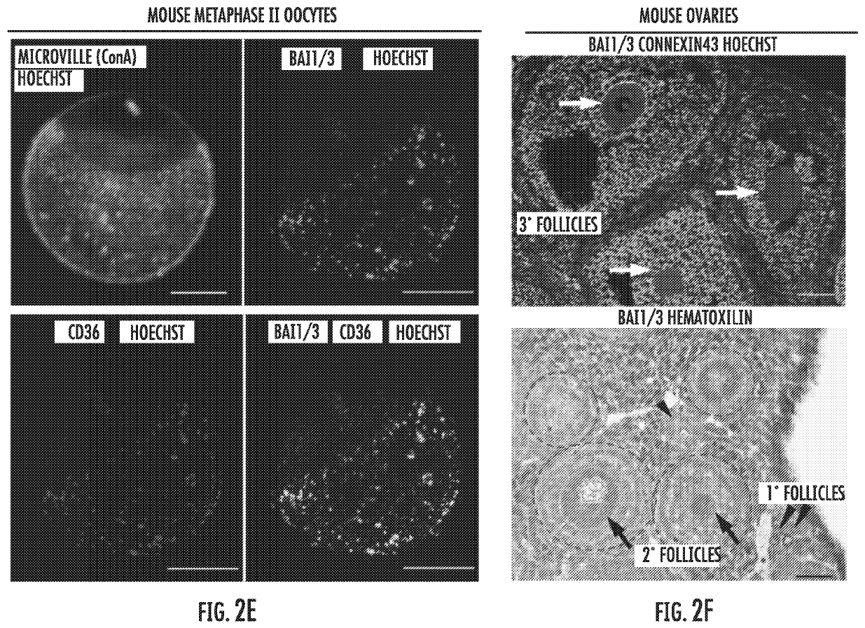

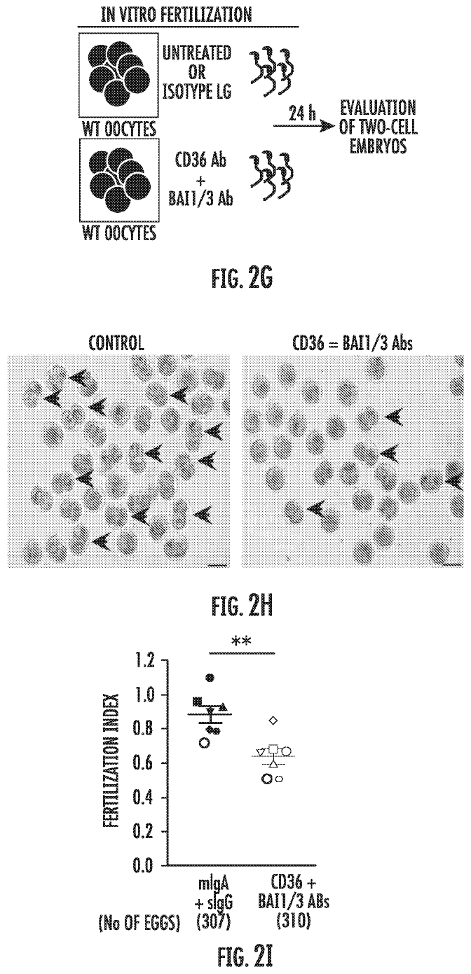

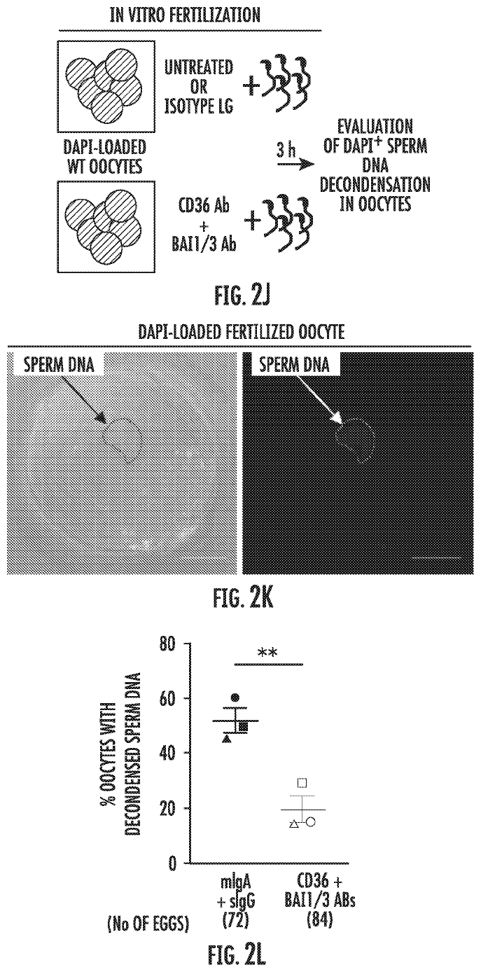

To test whether the surface of oocytes contains potential PtdSer binding sites, we took a simplified approach using the binding of the fluorescently labeled 2 μm carboxylate modified beads (2CMB), which are known to bind Annexin V and compete with PtdSer-exposing apoptotic cells²³. Zona pellucida-free oocytes were isolated from wild-type C57BL/6 female mice and were incubated with red-fluorescent 2CMB for 2 hours. At the end of the incubation period, the oocytes were stained with CD9 antibody to identify the microvillar region that is known to interact with sperm, fixed, and analyzed by microscopy (FIG. 2A). Oocytes readily bound multiple beads, and this interaction was restricted to the microvillus region of the oocytes (FIG. 2B). Importantly, this binding was significantly decreased when the beads were pretreated with the PtdSer masking agent BAI1-TSR (FIGS. 2B, 2C). Although PtdSer is an eat-me signal on apoptotic cells, extensive confocal sectioning of the bead-bound oocytes did not reveal obvious internalization of the beads under these conditions. These data indicated the existence of potential PtdSer binding molecules in the microvillar region on the surface of oocytes. As masking PtdSer on sperm significantly reduced the fertilization rate, we hypothesized that the PtdSer recognition receptors on the oocytes may contribute to the steps toward sperm:egg fusion.

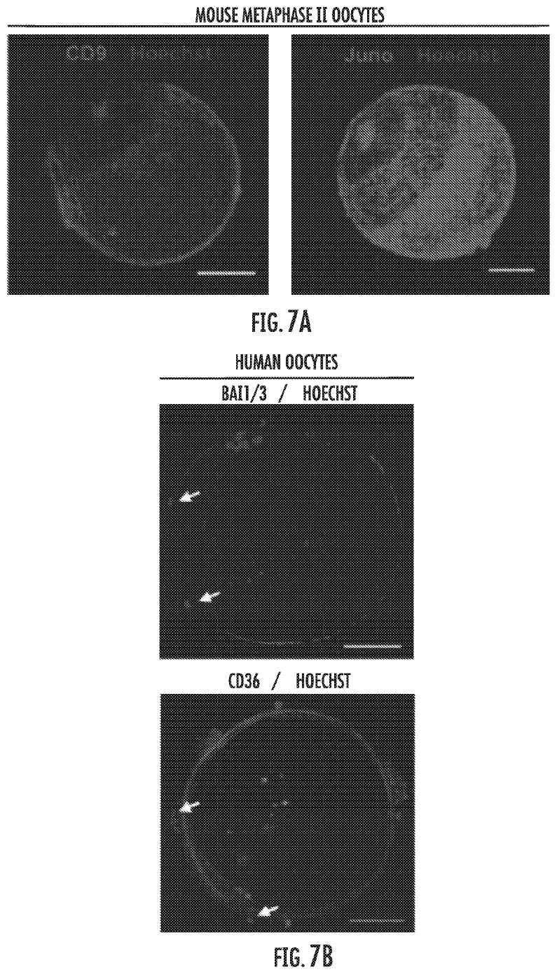

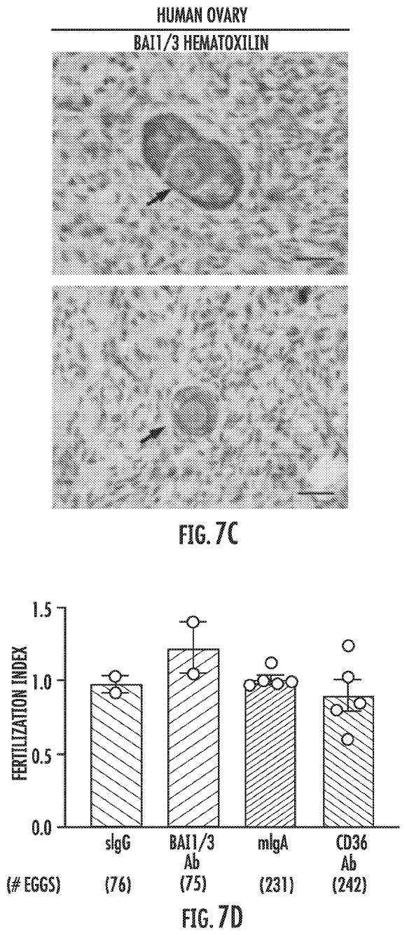

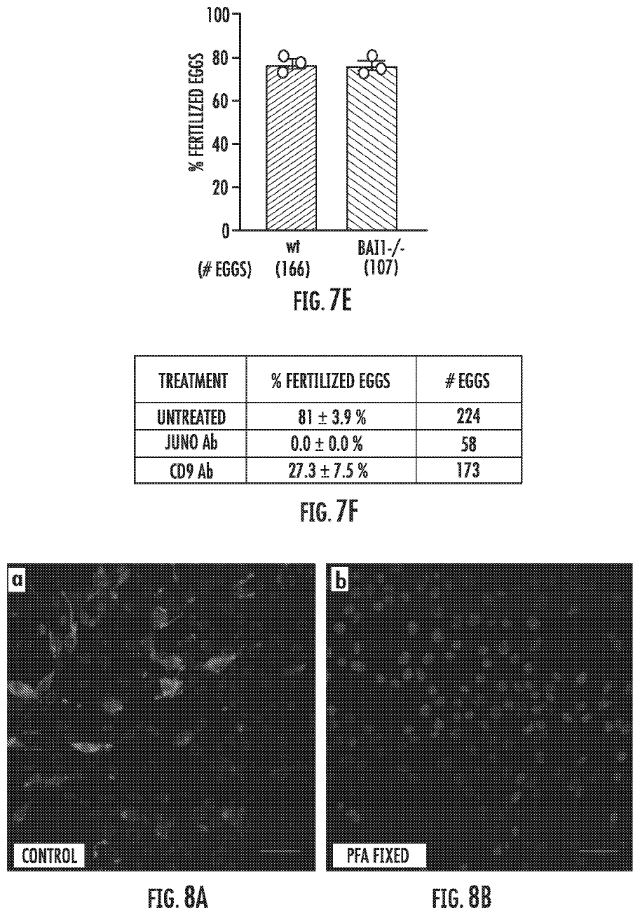

Several PtdSer recognition receptors with redundant functions have been identified on phagocytes to engage the PtdSer exposed on the apoptotic targets²³⁻²⁶. Therefore, we hypothesized that one or more such PtdSer recognition receptor(s) on the oocytes may engage the sperm during fertilization. In a previous bioinformatics analysis of oocyte genes, members of the BAI family as well as CD36 were reported to be expressed in both mouse and human oocytes²⁷. When we assessed the mRNA expression of BAI family members and CD36, we found readily detectable expression of BAI1, BAI3 and CD36 in mouse oocytes (FIG. 2D). BAI members belong to the type II adhesion family of GPCRs (hence also referred to as ADGBR family) with long extracellular region containing domains capable of directly binding PtdSer^{23, 25, 28-32}; CD36 is a member of the scavenger receptor family, and has also been linked to the binding of PtdSer^{24, 33-35} CD36 is also reported to function cooperatively with BAI1 on endothelial cells³⁶. Immunofluorescence microscopy using antibodies that recognize both BAI1 and BAI3 (referred to from here onwards as BAI1/3) or CD36, gave a prominent signal in the sperm-binding microvillar region (FIG. 2E); this staining pattern was also similar to the staining previously noted with concanavalin A³⁷ (FIG. 2E), Juno and CD9^{7, 9} (FIG. 7). When we assessed the expression of other known direct PtdSer binding receptors, we found detectable expression of the message for Timd4 but not Stab2 (FIG. 2D). Among the TAM family of receptors that can also recognize PtdSer (indirectly, via the bridging molecules Gas6 or protein S^{26, 38}), Mertk, but not Tyro3 and Axl were noted on oocytes³⁹. Immunohistochemistry of whole mouse ovaries revealed that BAI1 expression is detectable in oocytes from the earliest stages of folliculogenesis, with positive staining from primordial follicles through tertiary follicles (FIG. 2F). Similarly, we could readily detect staining for BAI1/3 and CD36 on human oocytes (discarded/unused oocytes acquired from clinical in vitro fertilization procedures) (FIG. 7B). Furthermore, expression of BAI1/3 on oocytes was detectable via immunohistochemistry on tissue sections of human ovary (FIG. 7C).

In the efferocytosis field, the PtdSer recognition by PtdSer receptors is known to include redundant mechanisms, as the charged head group of the lipid PtdSer can be recognized in a polyvalent fashion by multiple receptors to provide sufficient avidity and specificity^{38, 40, 41}. Therefore, we decided to test all of the five potential PtdSer receptors detected in oocytes—CD36, BAI1, BAI3, Tim4 and Mer-TK—via approaches that target them either singly or in combination. Interestingly, CD36 has been shown to cooperatively function with BAI1 in endothelial cells³⁶. Therefore, to test the potential multi-pronged interaction involving both CD36 and BAI1/3, we tested the effect of antibodies targeting CD36 or BAI1/3 (via antibody that recognizes both BAI1/BAI3), either alone or combination (see schematic in FIG. 2G). While antibodies to either BAI1/3 or CD36 alone did not inhibit fertilization (FIG. 7D), a combination of antibodies targeting both BAI1/3 and CD36 caused a reproducible and statistically significant inhibition of fertilization in vitro (FIGS. 2H, 2I). As a positive control, antibody to Juno could strongly inhibit fertilization (FIG. 7F). Next, we used a more direct assay for the sperm entry into oocytes. During fertilization, the nucleus of the sperm decondenses after entry into the oocyte cytoplasm⁴²⁻⁴⁴. This early step of fertilization can be scored using oocytes loaded with the DNA binding dye DAPI, and the appearance of DAPI-stained decondensed sperm DNA (FIG. 2J)⁴²⁻⁴⁴. After establishing and validating this assay, we tested the efficacy of the BAI1/3 and CD36 blocking antibodies on the sperm entry into the oocyte. We found a 67% decrease in the percentage of oocytes with decondensed sperm DNA (FIGS. 2K, 2L), suggesting that blocking three of the five PtdSer receptors (BAI1/BAI3, and CD36) expressed on oocytes can also impair fertilization in vitro, complementary to the masking of PtdSer on the sperm.

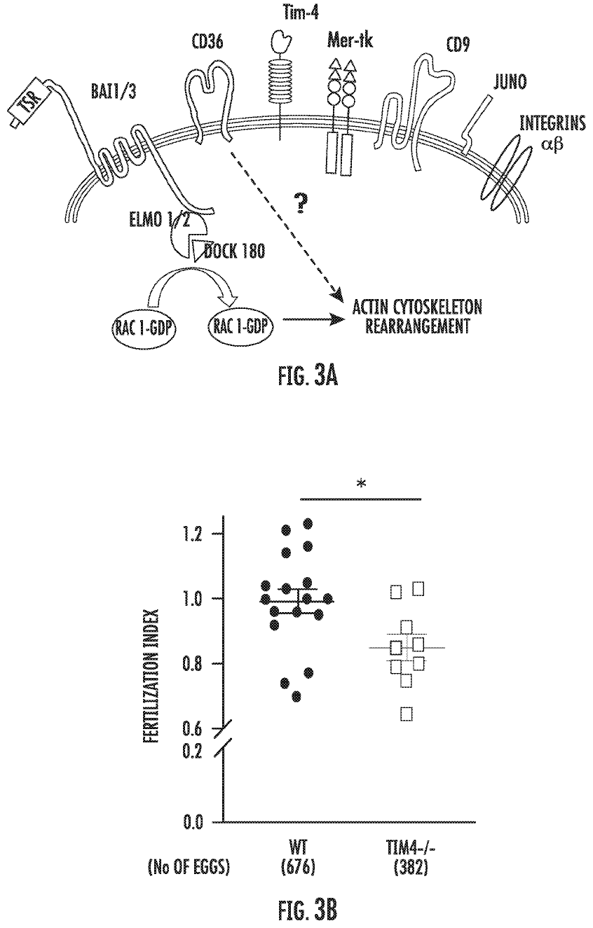

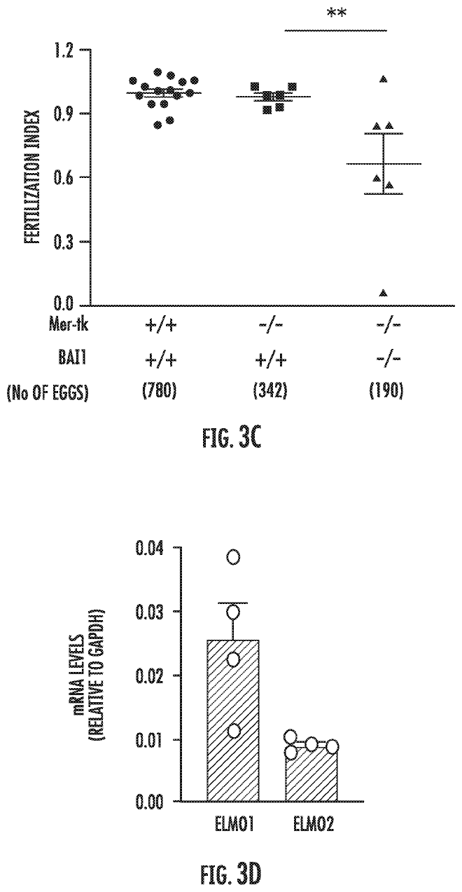

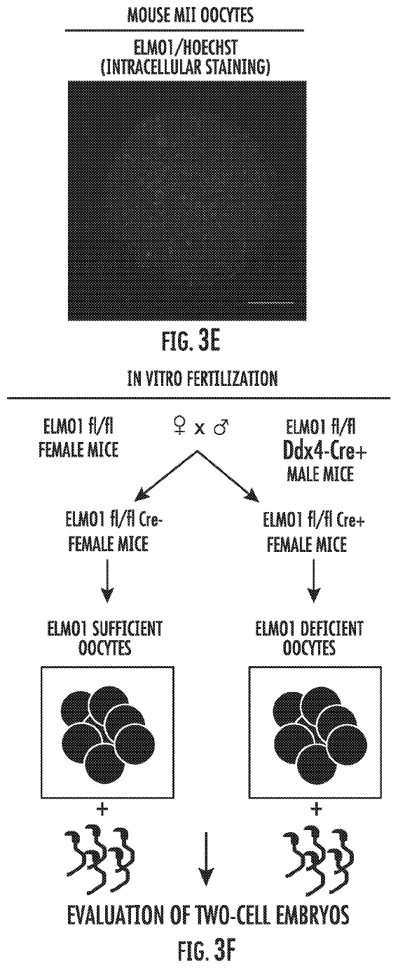

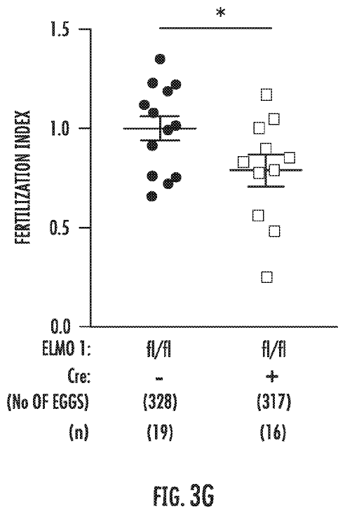

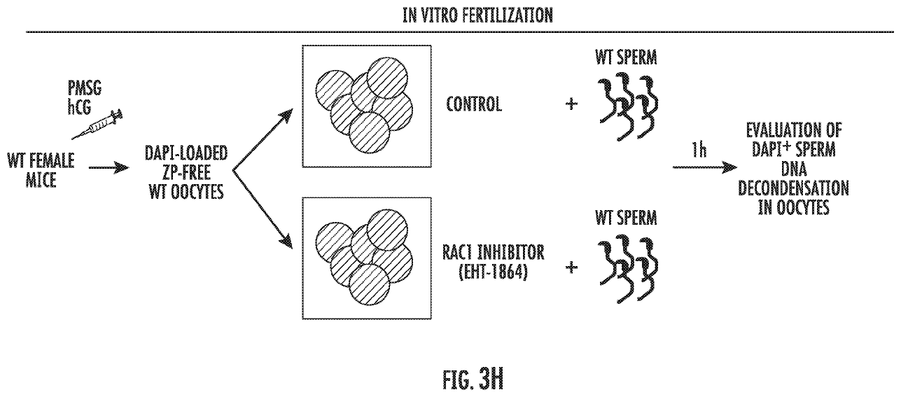

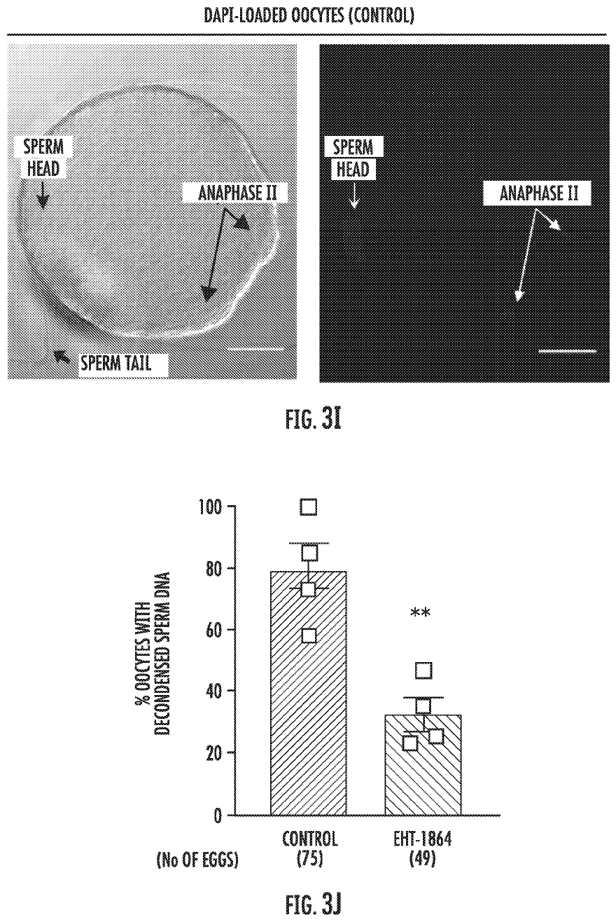

Next, we wanted to genetically test the contribution of oocyte PtdSer receptors to fertilization. Because of the extensive functional redundancy among PtdSer recognition receptors it is widely reported that single knockout of PtdSer receptors often show partial defects in apoptotic cell clearance, and defects are better revealed by deletion of more than one receptor⁴⁵⁻⁴⁸. We tested three of the PtdSer receptors expressed on oocytes using single or double knockout mice: Tim-4, BAI1 and Mer-TK. Tim-4 directly binds PtdSer while Mer-TK binds PtdSer indirectly through the bridging molecules Gas6 or Protein S [note: oocytes also express Gas6³⁹]. Mice deficient in Tim-4 alone showed a modest but statistically significant reduction in the percentage of fertilized eggs (FIG. 3B). We then tested the role of Mertk and Bai1 genetically. We performed in vitro fertilization assays with oocytes isolated either from Mertk^{-/-} or Bai1^{-/-} mice, or mice double deficient for both Mertk and Bai1. While oocytes isolated from single deficient mice (either Mertk^{-/-} or Bai1^{-/-}) could be fertilized similar to wild type eggs (FIG. 3C and FIG. 7E), the double deficient Mertk^{-/-}Bai1^{-/-} oocytes show a significant reduction in fertilization (FIG. 3C). Collectively, these data suggest that even with the considerable redundancy among the PtdSer receptor family, a statistically significant effect can be observed in in vitro fertilization with oocytes deficient in specific PtdSer recognition receptors.

### Example 3

### The BAI1/3-ELMO1-RAC1 Signaling Axis Affects Fertilization

With respect to signaling downstream of PtdSer recognition receptors, CD36 has a rather short cytoplasmic tail without an obvious direct signaling, and CD36 can cooperatively signal with BAI1³⁶. Among the PtdSer receptors, signaling downstream of the BAI family members is one of the best characterized^{23, 25, 49}. Both BAI1 and BAI3 have long cytoplasmic tails that associate with the adapter proteins ELMO1 and/or ELMO2 (depending on the cell type), with subsequent signaling (in complex Dock family proteins) and activation of the small GTPase RAC1^{23, 49-52}. GTP-bound active RAC1 promotes actin cytoskeletal remodeling during adhesion, phagocytosis, and cell:cell fusion events (FIG. 3A). In oocytes, we detected both Elmo1 and Elmo2 mRNA, with Elmo1 expression higher than Elmo2 (FIG. 3D). At the protein level, ELMO1 expression was readily detected by immunofluorescence in isolated oocytes (FIG. 3E). To assess the importance of ELMO1 in fertilization, we crossed mice carrying floxed Elmo1 alleles (Elmo1^{fl/fl}) with Ddx4-Cre mice⁵³, which express the Cre recombinase specifically in oocytes from the earliest stages (FIG. 3F). We super-ovulated the Ddx4-Cre/Elmo^{fl/fl} female mice, isolated the oocytes, and performed in vitro fertilization assays using caudal sperm from wild type mice. We noted a significant reduction in in vitro fertilization with oocytes from Ddr4-Cre/Elmo1^{fl/fl} mice, compared to control mice (FIG. 3G). The partial reduction is consistent with the continued expression of ELMO2, which can substitute for ELMO1¹¹. Incidentally, female nematodes lacking the ELMO homologue ced-12 have been shown to have lower fecundity, with fewer progeny produced s.

ELMO proteins (together with Dock family members) function as upstream activators of the small GTPase RAC1, which regulates actin cytoskeletal rearrangements⁵¹ (FIG. 3A). Since genetically testing the requirement for RAC1 is not feasible due to the role of RAC1 during oocyte development and other steps after the sperm entry⁵⁴, we took a pharmacological approach and used the sperm DNA decondensation assay to more directly score the sperm entry into oocytes. We treated oocytes with the RAC1 inhibitor EHT-1864 (see methods) and also added EHT-1864 during co-incubation of sperm and oocytes (note that both sperm and oocytes were harvested from wild type mice) (FIGS. 3H, 3I). EHT-1864 caused a significant reduction in the number of oocytes with decondensed sperm DNA (FIG. 3J). Importantly, this effect did not appear to be due to the RAC1 inhibitor affecting sperm, as sperm incubated with EHT-1864 alone under the assay conditions showed no reduction in the motility (FIG. 3K). These data suggest that oocyte BAI1/3 and CD36, as well as the ELMO-RAC1 signaling module downstream of BAI1/3 contribute to the functional steps of fertilization.

### Example 4

### PtdSer-Dependent Fusion of Sperm with Skeletal Myoblasts

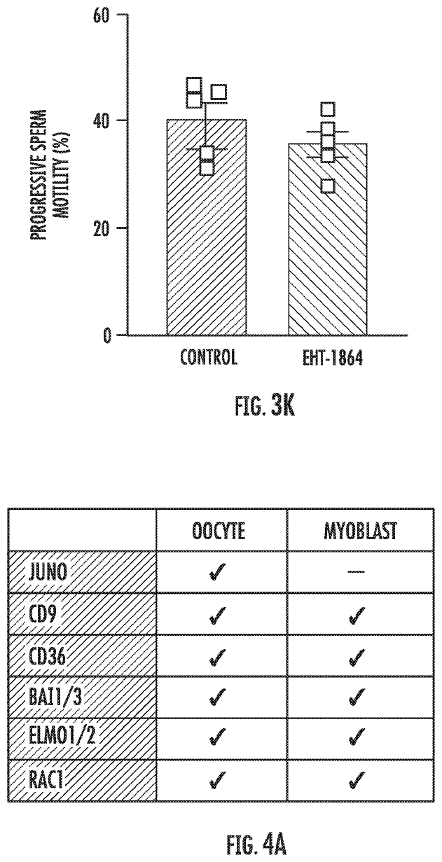

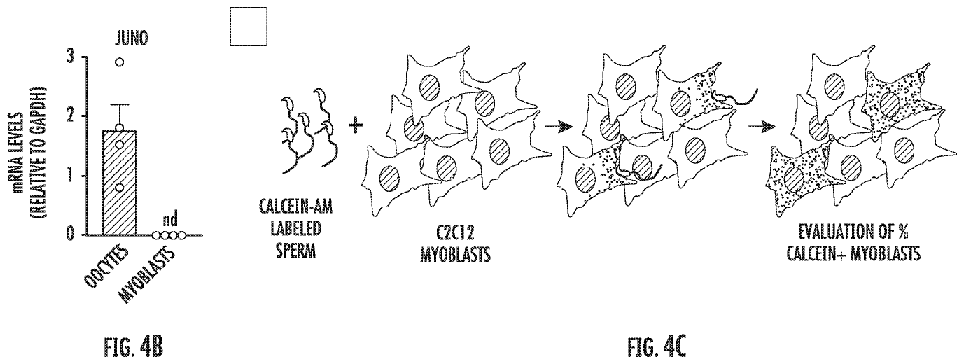

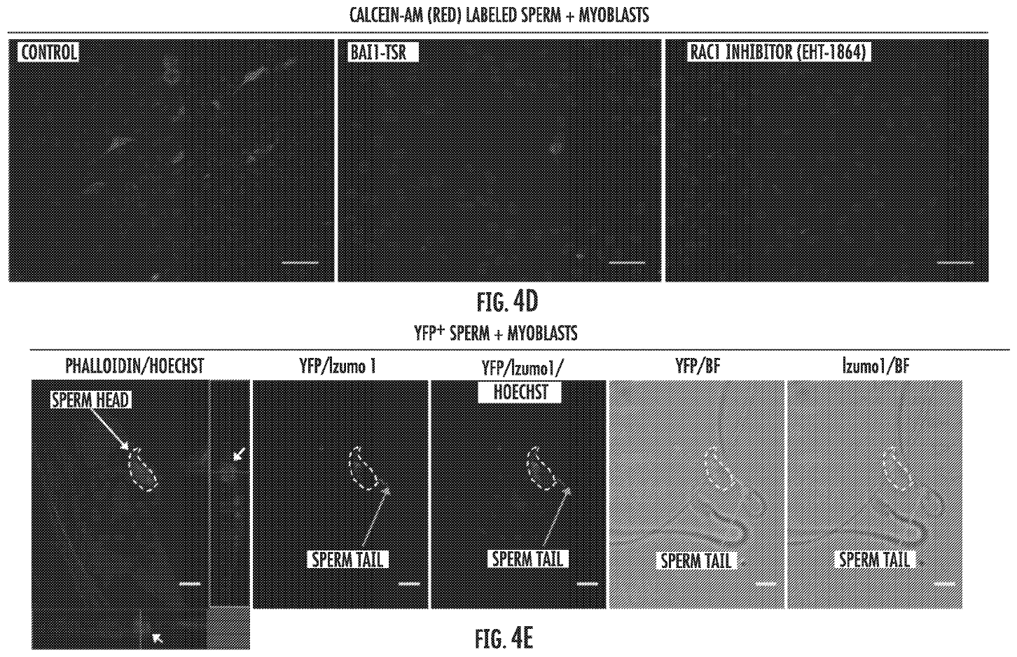

Our results up to this point suggest that PtdSer on the sperm and its receptors BAI1/3, CD36, Tim-4 and Mer-TK on the oocyte can promote fusion via the ELMO-RAC1 signaling pathway. Our lab and others^{19, 25, 49} have previously demonstrated that PtdSer exposure on skeletal myoblasts is important for the fusion between myoblasts to form myotubes, and this occurs in a BAI1/3-ELMO-RAC1 dependent manner^{19, 25, 49, 55}. Intriguingly, when we examined the expression of genes linked to the sperm:egg fusion in myoblasts, we found that oocytes and myoblasts both expressed the membrane proteins CD9, CD36, BAI1, and BAI3, as well as cytoplasmic ELMO2, and RAC1 (FIG. 4A)^{8, 25, 49, 56-59}, while Juno expression was not detected in myoblasts (FIG. 4B). Therefore, we asked whether caudal sperm could fuse with skeletal myoblasts, as it has been shown with other somatic cells^{60, 61}, and whether this Juno-independent fusion was mediated by the BAI1-ELMO1-RAC1 module expressed by myoblasts.

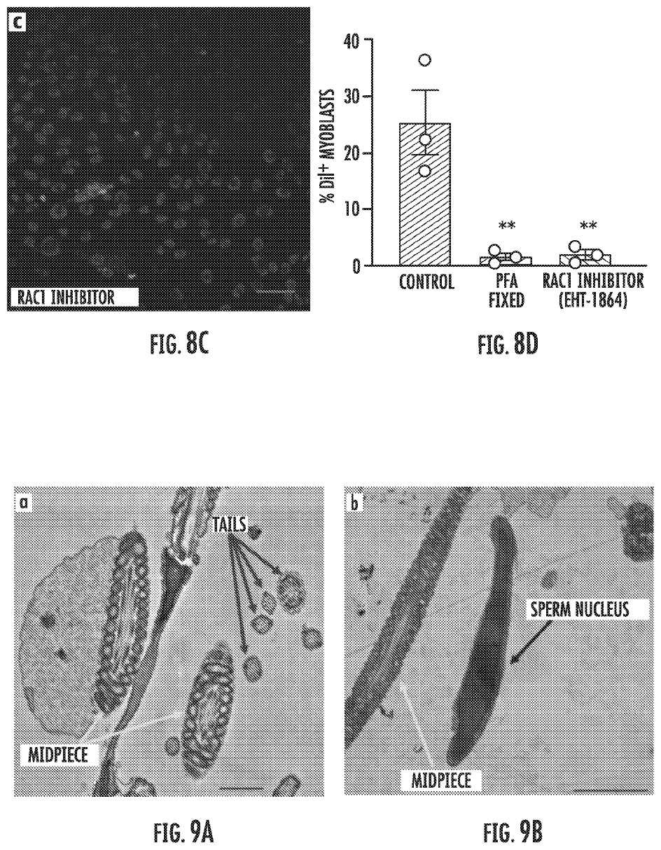

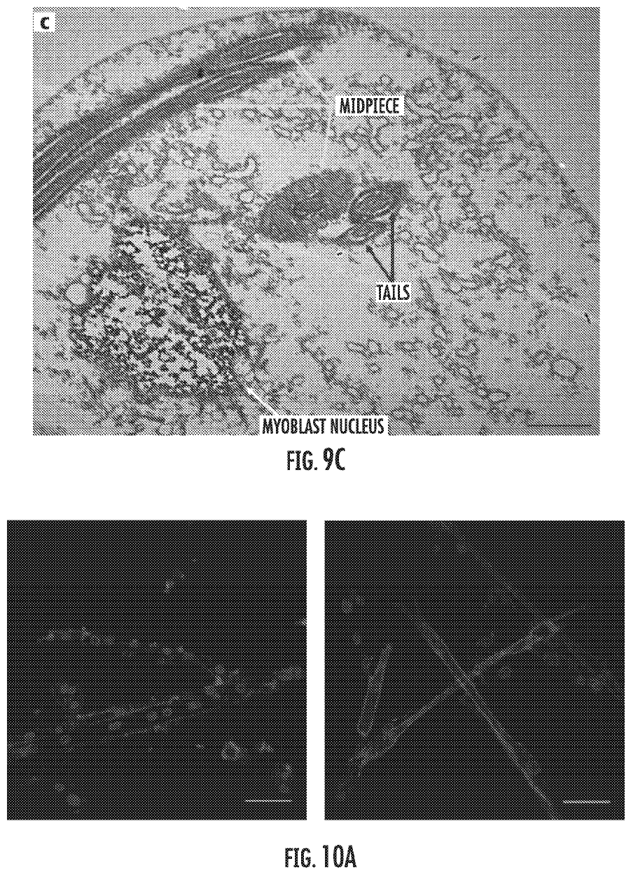

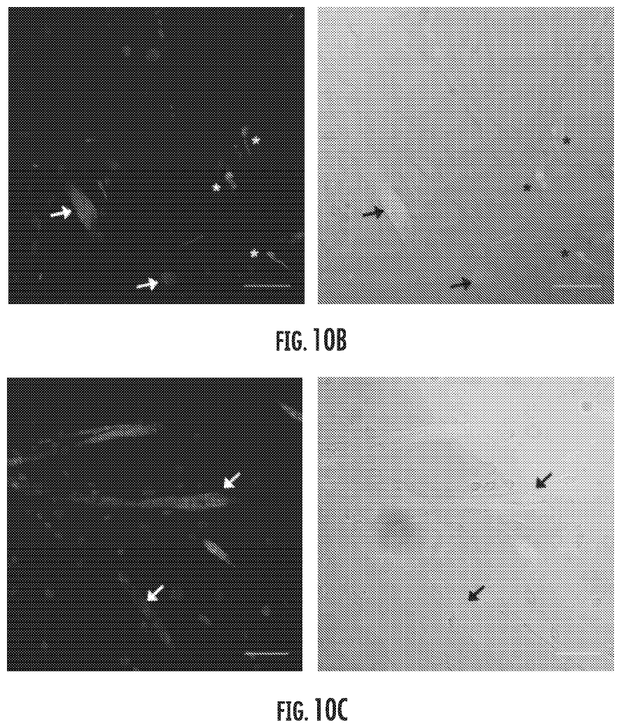

We labeled caudal sperm with a red-fluorescent cytoplasmic dye (Calcein-AM)⁶⁰, incubated them with C2C12 mouse myoblasts, and looked for myoblasts that acquire the sperm-derived Calcein-AM staining (FIG. 4C). Of note, there was no myoblast:myoblast fusion when they were in growth medium in a non-confluent state. However, some of these myoblasts are known to be poised for fusion²⁵. Remarkably, we could readily detect transfer of sperm-derived Calcein-AM into few of the myoblasts in a quantifiable manner (FIG. 4D). To further address the fusion between sperm and skeletal myoblasts, we took three additional approaches. First, labeling sperm with another dye (DiI) produced similar results as scored by fusion with myoblasts (FIG. 8). Second, we isolated sperm from mice expressing transgenic YFP and, after incubating them with myoblasts, we detected for YFP/GFP and the sperm specific protein Izumol within the fusing myoblasts by immunostaining. We could readily observe the signal for YFP, and the sperm specific protein Izumol, in addition to the DNA from the sperm head within the myoblasts (FIG. 4E). As a third approach, we used electron microscopy to detect the presence of sperm within myoblasts. The midpieces (containing multiple mitochondria), and the tails from multiple sperm could be detected inside the cytoplasm of a myoblast (FIG. 9C). Of note, we do not detect any obvious membrane surrounding the sperm structures, suggesting that the sperm is not contained within a phagocytic vesicle. Interestingly, aminophospholipid asymmetry on myoblasts differs from fibroblasts¹⁶, and this may, in part, explain sperm fusion with myoblasts but not fibroblasts. Since C2C12 myoblasts are an immortalized cell line, we also tested whether sperm could fuse with cultured mouse primary myoblasts, which also express BAI1/3 and CD36 (FIG. 10A), and this was indeed the case (FIGS. 10B, 10C). Further, when we incubated sperm with primary bone marrow derived macrophages, the sperm-derived Calcein-AM was not dispersed within the cytoplasm of macrophages as was the case with myoblasts, but rather the sperm appeared to be phagocytosed by the macrophages (FIG. 11).

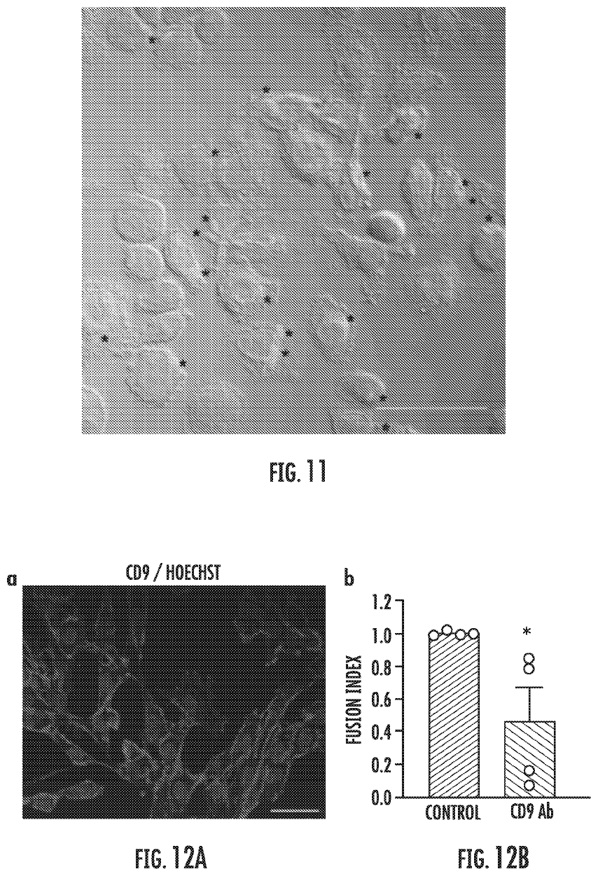

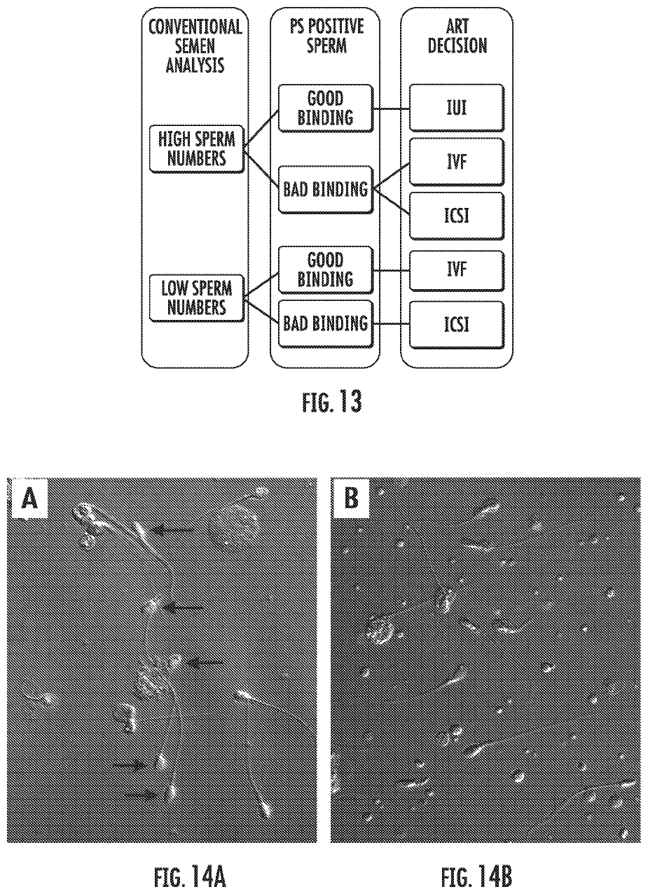

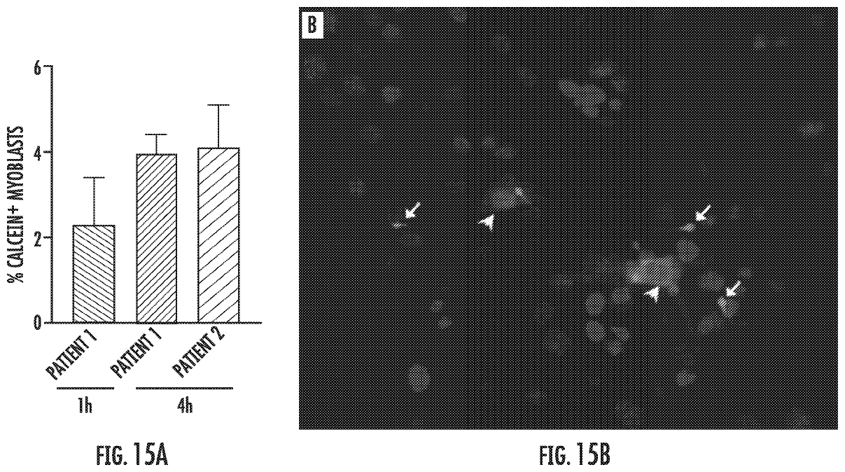

We next asked whether this sperm:myoblast fusion event also depends on PtdSer exposure on the sperm and the BAI1-ELMO-RAC1 module on the myoblast. First, blocking PtdSer on the sperm (via BAI1-TSR) significantly decreased the fusion of sperm to myoblasts (FIGS. 4D, 4F). Second, antibody mediated blocking of BAI proteins [BAI3 is expressed at a much higher level than BAI1 in C2C12 myoblasts⁴⁹] potently blocked sperm fusion to myoblasts (FIG. 4F). Third, C2C12 myoblasts with knockdown of Elmo2 [the predominant ELMO isoform expressed in C2C12 myoblasts⁴⁹] showed a significantly reduced Calcein-AM acquisition from the labeled sperm (FIG. 4F). Fourth, the RAC1 inhibitor EHT-1864 also potently blocked the sperm:myoblast fusion (FIGS. 4D, 4F and FIG. 8). Consistent with the BAI1/3-ELMO-RAC1 module being involved in cytoskeletal rearrangements in cell:cell fusion during myotube formation, the treatment of myoblasts with cytochalasin D, or fixation of myoblasts with paraformaldehyde, prior to adding the sperm abrogated the sperm-derived fluorescence acquisition by myoblasts (FIG. 4F and FIG. 8). These data suggest that PtdSer exposure on viable sperm and its interaction via the BAI3-ELMO-RAC1 module on myoblasts can contribute to cell:cell fusion, in a Juno-independent manner. Since both myoblast:myoblast⁵⁶ and myoblast:sperm fusion (FIGS. 12A and 12B) are also dependent on CD9, another surface molecule relevant in sperm:egg fusion^{8, 62}, it is possible that PtdSer receptors may work together with CD9 during sperm:myoblast fusion. Although this system scores fusion between a diploid myoblast and a haploid sperm (i.e. non-productive), this could potentially serve as an assay to test sperm competency among infertile couples. Moreover, C2C12 myoblasts are easier to manipulate and genetically modify compared to oocytes, and provide a good system to probe the mechanistic aspects of sperm fusion.

### Discussion of Examples 1-4

Taken together, the data presented in this work provide several insights relevant for sperm:egg fusion during mammalian fertilization. Using different approaches the present data demonstrate that the heads of viable and motile sperm display PtdSer. PtdSer asymmetry on the leaflets of plasma membrane is orchestrated in a complex way, via multiple proteins functioning in opposite directions; further, PtdSer is also essential for many intracellular trafficking events. Deletion of genes coding for enzymes that are required for PtdSer synthesis, Ptdss-1 and Ptdss-2, leads to embryonic lethality⁶³. As mice for conditional targeting of Ptdss-1 or Ptdss-2 genes are not currently available, we took the approach of masking or competitively interfering with the PtdSer on sperm. This led to reduction in in vitro fertilization ranging from 40-85% with three different PtdSer targeting agents (including Annexin V, the highest affinity reagent, with the best effect). From the oocyte end, blocking of individual PtdSer receptors in isolation often had modest effects, likely due to well-known redundancy among PtdSer receptors in phagocytosis studies; however, combinatorial targeting multiple PtdSer receptors via antibody-mediated blocking, genetic deletions, and disruption of cytoplasmic signaling approaches suggest a role for the PtdSer receptors in sperm entry into oocytes. Mechanistically, analogous to the role of PtdSer and its receptors in cell:cell fusion in other systems, the presently disclosed data suggest that PtdSer on sperm, PtdSer receptors and the downstream signaling molecules in the oocyte play a role in sperm:egg fusion.

In the field of fertilization, ablation of in vivo fertility after genetic deletion has become the gold standard to demonstrate the requirement for molecules critical in fertilization. This has been only been met for a few genes coding for proteins such as Izumol, Juno, and CD9. This criterion was difficult to meet in the present study because PtdSer is a lipid on cell membranes, and consistent with the myriad roles in normal cellular functions, and the complex number of steps that control PtdSer asymmetry, genetically achieving a PtdSer-null mature sperm (expecting to develop normally through the various steps of spermatogenesis) is currently unfeasible. As PtdSer receptors are also highly redundant, generation of mice lacking 3, 4, or 5 different PtdSer also poses problems. Thus, the technical issue of genetically manipulating PtdSer exposure and its recognition remains to be tackled in the future. To assuage this issue, we have tested the roles of both PtdSer and its receptors from both the sperm and the oocytes sides, using multiple in vitro approaches (in contrast to previous works, where either ligands on the sperm or the oocyte were studied independently). Importantly, after blocking either the ligand on the sperm or the receptors/signaling pathway on the oocytes, we observed similar outcomes.

It is posited that Izumol and PtdSer on sperm could function cooperatively in mediating sperm binding to oocytes and fusion. This is based on the known properties of both PtdSer and Izumol. When PtdSer acts as an eat-me signal or as a fusion signal, it is not sufficient by itself, and requires additional players for its functionality^{45, 64}. Further, caudal sperm already exposes PtdSer, while Izumol is exposed on caudal sperm after the acrosome reaction⁴; thus, both Izumol and PtdSer can be present on the sperm surface at the same time for subsequent interactions with the oocyte. We also envision multimeric interactions between the sperm and egg surfaces for achieving sufficient specificity and avidity. For example, Izumol is known to dimerize^{7, 65}; similarly, PtdSer binding often involves multiple receptors and cooperative polyvalent binding^{40, 64}. Based on the Izumol:Juno interaction, we anticipate that this may provide the initial strong gamete binding, followed by the interaction between PtdSer on sperm and the PtdSer receptors on oocytes. At this point, the BAI1/3-ELMO-RAC1 module, along with other molecules that have been previously linked to fertilization on the oocytes (such as CD9), may help achieve gamete fusion. These hierarchical/step-wise interactions between the gametes leading to fusion are in part supported by the sperm fusion with myoblasts (that are poised for fusion) in the absence of Juno. This type of combinatorial use of diverse molecules to achieve cell:cell binding/subsequent signaling is very analogous to interactions within the immune system: e.g. during T cell interaction with an antigen presenting cell, the central driver is the TCR:MHC/peptide interaction; yet, a host of other accessory molecules are fundamentally important for achieving specificity and optimal activation of T cells after antigen recognition. Similar to Juno (which is GPI-anchored and does not have its own signaling motif), the TCR does not have a signaling motif of its own and requires associated molecules for inducing downstream signaling and activation. Analogously, the PtdSer recognition receptors on the oocytes may work together with Juno in initiating intracellular signaling within oocytes, eventually leading to gamete fusion. In summary, these data suggest that PtdSer on sperm and its receptors on oocytes as functional players that can work in conjunction with Izumo and Juno to promote sperm:egg fusion during fertilization.

### Example 5

### Assessment of Human Sperm

Human sperm stain positive for PS. Human sperm from a male with known fertility were retrieved, washed, and stained with Annexin V and DAPI, as described above. The head region of the sperm displayed strong positive staining for PS. See FIGS. 14A and 14B. FIGS. 14A and 14B show that PS is exposed on human sperm, with merged images of human sperm stained with annexin V (red) and DAPI (blue). Arrows indicate PS positive sperm from (FIG. 14A) a fertile male (normal semen analysis; proven paternity) and (FIG. 14B) from an infertile male (abnormal semen analysis; no offspring). These results demonstrate that human sperm do stain positive for PS using Alexa-Fluor 568-conjugated annexin V (FIG. 14A).

In further testing, 20 human samples from males with known fertility are selected to assess buffers, PS staining reagents, and methods for detecting PS on human sperm. Staining reagents and methods that stain PS on human sperm are confirmed and a lower limit of detection (LLOD) based on the number of sperm cells required and the concentration of reagents needed to identify PS-positive human sperm is identified

The human semen samples are obtained from men with known fertility and a normal semen analysis (WHO standards) and are obtained from the clinical urology lab. Approximately half of the sample volume come directly to the lab for PS staining, while the remaining half undergo a basic semen analysis. Knowing the results of the basic semen analysis is helpful in studies to correlate PS expression with other semen parameters. Staining for PS with annexin V, anti-PS antibody, and BAI1-TSR. Appropriate controls will be used for each reagent, as we have done in our mouse studies described

Briefly, to stain sperm with annexin V, sperm are dispersed, counted, washed, and stained with either Alexa-Fluor 568-conjugated annexin V or PS Ab conjugated with Alexa Fluor 488. Apoptotic thymocytes are obtained according to known techniques and used as a positive control. The sperm and apoptotic thymocytes are analyzed with an LSM 700 confocal microscope. A similar staining method is used for staining sperm with BAI1-TSR, and GST is used as a control (as described elsewhere herein). In all methods, the concentration of reagents used in our mouse studies is the starting point. If staining is inadequate, adjustments are made as necessary. Once the proper concentrations of reagents are optimized, the numbers of human sperm cells to be stained are adjusted. 10-fold dilutions are used until a lower limit of detection (LLOD) for each of the three PS binding agents is reached. All results are compared as well as overall ease of use, cost of reagents, and time to stain, to assess PS-staining reagents and staining protocols for demonstrating PS surface expression on human sperm. Annexin V is expected to be a reagent of choice for staining human sperm for PS exposure based on the high affinity of annexin V for PS (K_{D}=6.6 nM), the simplicity of the one-step staining procedure, and the relative lower cost.

Additionally, a larger cohort of human samples (˜75) from a more diverse group of males with known and unknown fertility is obtained to evaluate the test's applicability, ease of use, and performance characteristics. In addition, a software program is provided to quantify the ratio of PS+ sperm to the total number of sperm in the sample. After optimizing reagents and methods for screening human samples and increasing sample size to reduce any margin of error and provide a more precise estimate of the performance parameters, samples are retrieved from men that are diagnosed infertile via an abnormal semen analysis and lack of paternity. The percentage of PS-positive sperm within each sample is determined as a step in the assessment.

A purpose for collecting these samples is to observe potential variance in PS levels, which facilitates demonstration of feasibility. The samples are retrieved from a clinical urology lab and a full semen analysis is done on each sample. Samples are characterized based on the 2010 WHO guidelines of semen analysis. With concentrations of reagents and procedures determined as described herein, the percentage of sperm cells that are positively stained for PS is determined. After PS staining, sperm are stained with DAPI to label the DNA. The cells are then placed on slides and 6-8 photographs taken per sample. The percentage of annexin V-positive sperm are then counted. Work in the mouse model showed that approximately 40% of sperm from the cauda epididymis are positive for PS, as described elsewhere herein. Data with human samples suggests that approximately 45% of human ejaculated sperm from fertile men with normal semen analysis are PS positive (N=3; FIG. 14A) and sperm from an infertile male (low sperm numbers, no paternity) revealed the absence of PS-positive sperm (FIG. 14B).

An image-quantifying software is also developed using the binary mask of the DAPI-labeled sperm heads as a basis for analysis, since it includes all the sperm in the sample. For each sperm within the image, the overlap between the sperm head (mask) and the PS-stained content is measured. Sperm are labeled positive if the overlap between DAPI and PS label is above 30 percent. This software has been used to compare over 600 sperm images using labeled mouse sperm. Thus, the software program can calculate the ratio of PS-positive sperm. Results provide an early indication of the percentage of PS-positive sperm in a normal semen analysis and if this percentage differs in men that are infertile. Due to an n of 75 in the sample set, conclusions at this time are not over emphasized.

### Example 6

### PS-Dependent Human Sperm:Myoblast Fusion Assay

Data demonstrates feasibility of the human sperm:myoblast fusion assay. See FIG. 4. As detailed elsewhere herein, a challenge in the field in assessing sperm quality in infertile couples is the lack of a simple, short-term functional assay that can assess the ability of the sperm to fuse with oocytes. Thus, this Example relates to a sperm:myoblast fusion assay for human sperm.

In accordance with approved protocols human sperm samples, e.g., 20 samples, are collected for this analysis. The samples are retrieved from a clinical urology lab and a full conventional semen analysis is conducted on each sample. Samples are established as “normal”, as determined by the 2010 WHO guidelines of semen analysis. Human sperm samples are tested for the uptake of the fluorescent intracellular dyes Calcein-AM and DiI. In brief, sperm are allowed to swim in TYH+0.75 mM Methyl-beta-cyclodextrin (MBCD)³⁷ for 15 min, then counted and stained with 4 μM Calcein red orange-AM (Invitrogen) for 30 min in PBS, using known staining techniques. Cells are washed, counted again, and resuspended in DMEM medium supplemented with 20% heat-inactivated FBS. For the fusion assay, C2C12 murine skeletal muscle myoblasts (ATCC) are maintained at sub-confluent densities in DMEM medium supplemented with 20% heat-inactivated FBS. Cells are trypsinized, counted, and plated in 4-well LabTek II Permanox chamber slides (25,000 cells/well). After 24 h, myoblasts are treated with appropriate buffer alone or 5 μg/mL cytochalasin D (Sigma), for 30 min at 37° C., 8.5% CO₂ prior to the sperm co-incubation. Calcein-AM-positive sperm (250,000 sperm/well) are added and incubated for 4 hr at 37° C., 8.5% CO₂. After the 4 hr co-incubation, myoblasts are washed, stained with Hoechst, and mounted. Cells are analyzed the same day and 6-8 fields/well photographed using the Axio Imager 2 with Apotome (Zeiss). Image J cell counter plugin software is used to quantitate stained myoblasts. The fusion index is calculated as the percentage of Calcein AM-positive myoblasts in the experimental group normalized to control untreated cells. Results provide representative concentrations of the staining reagents, sperm, and myoblasts to be used in this fusion assay. It is expected that Calcein-AM is a reagent of choice for staining human sperm, based on the simplicity of the one-step staining procedure.

With the optimization of the reagents and methods now adjusted to screen human samples, sample sized is increased. Increasing sample size aids in decreasing any margin of error between samples and in providing a more precise estimate of the parameters. A larger cohort of human samples (75) from a more diverse group of males with known and unknown fertility is used to evaluate applicability of the assay, ease of use, and performance characteristics. In addition, a software program is provided and used to quantify the percentage of positively-stained myoblasts per number of sperm added to determine the fusion index.

In the larger cohort, human samples from a more diverse group of males with known (i.e. fertile or infertile), and unknown fertility are provided to test the assay's applicability, ease of use, and performance characteristics. The samples are retrieved from a clinical urology lab, as described above, and a full conventional semen analysis is conducted on each sample. The image-quantifying software discussed above is employed as needed. The “expected” percentage of positive myoblasts after incubation with the labeled sperm, i.e., the fusion index, is determined. A sample size of 75 might be small but should be sufficient for demonstrating fluctuations between samples of variable fertility.

### Example 7

### Staining for PS as an Indicator of Fertilization Competent Sperm (FCS)

Previous methods considered cells that stained positive for Annexin V were apoptotic (dead). It has been shown in accordance with the presently disclosed subject matter that sperm cells that stain positive for Annexin are not only alive, but that these cells are the sperm capable of fusing with an egg.

In some embodiments, the presently disclosed subject matter provides methods for determining fertilization competent sperm (FCS) in a semen sample. In some embodiments, the methods comprise:

1. Isolate sperm

2. Stain for using either PS: a) Annexin V and/or b) BAI-TSR-GST (comprising SEQ ID NOs: 1 and 2).

3. Stain for total cell number

4. Stain for apoptotic and dead cells

5. Count PS positive cells minus dead cells and calculate percent PS in sample. In some embodiments, the presently disclosed subject matter provides that fertile samples contain at least about 50% PS+ cells.

A. Representative Protocol for Staining Bull (Bovine) FCS

This protocol can also be used for the staining of sperm from other species, e.g., equine, canine, feline, avian, etc.

Day 0:

1. Prepare TYH+BSA and keep in incubator 37° C., 5% CO₂ overnight.

Day 1:

1. Prepare tubes with ˜200 ul TYH+BSA (keep in incubator).

2. Transport sperm straws in dry ice.

3. Thaw for ˜1 min at 35° C. (water bath).

4. Cut crimped end and let sperm diffuse into the tube with 200 ul of TYH+BSA

5. Incubate for 5 min in incubator.

6. Count (1:10 dilution).

7. Take 0.5×10.6 cells and transfer to another tube (“FACS tubes”).

8. Add 500 ul/tube of 1× binding buffer and centrifuge 6 min at 1400 rpm.

9. Aspirate supernatant very carefully.

10. Annexin V staining:

Add ˜90 ul 1× Binding buffer and 10 ul of Annexin V (Alexa Fluor 568). Incubate for ˜12 min at room temperature and dark.

Staining necrotic cells: 7AAD: separate from Annexin V (or use Annex FITC).

Add ˜90 ul 1× Binding buffer and 2 ul of 1:20 7AAD (prepared in 1× binding buffer).

11. Add Hoechst: add 5 ul/tube from a dilution 1:100 prepared in 1× binding buffer. Incubate ˜3-4 minutes extra.

12. Wash: add ˜500 ul 1× binding buffer and centrifuge 6 min at 1400 rpm.

13. Aspirate supernatant very carefully and not completely.

14. Disperse on a slide, trying to have a thin layer. Add coverslip trying to avoid bubbles.

15. Take photos.

Smears for cleaved caspase 3 (CC3) staining (apoptotic cells):

Fix sperm in 4% formaldehyde: add ˜100 ul to ˜100 ul of sperm (final concentration: 2%). Dilute in 1× Binding buffer.

Let dry and keep at 4° C. until immunofluorescence is performed.

B. Representative Protocol for Staining Human FCS

1. Pick up sample (semen) from clinical lab.

2. Count sperm numbers using Neubauer hemocytometer (dilute 1:10-1:20, samples varies).

3. Take 0.5×10⁶ sperm/tube for staining with 1) Annexin V, 2) 7AAD and 3) CC3.

4. Enrich sperm using “Isolate gradient” (Irvine 99275 Isolate):
1) Warm 2 ml of isolate in a 15 ml tube (37° C., waterbath).
2) Place 2 ml of semen and gently layer over the isolate gradient.
3) Centrifuge for 20 min at 350 g.
4) Remove the gradient and the seminal fluid trapped within the gradient. Slowly aspirate media as you approach the pellet. Do not disturb the pellet.
5) Wash with media (usually use TYH or TYH+BSA).
6) Centrifuge for 8 min at 350 g.
7) Aspirate media down to the pellet and resuspend in media (TYH+BSA).
8) Count #sperm and stain for Annexin, 7AAD or CC3 or keep overnight in incubator (37° C., 5% CO₂) for myoblast fusion.

Annexin V Staining (PS+ Cells):
Add ˜90 ul 1× Binding buffer and 10 ul of Annexin V (Alexa fluor 568).
Incubate for ˜12 min at room temperature and dark.

### 7AAD Staining (Necrotic Cells):

Add ˜90 ul 1× Binding buffer and 2 ul of 1:20 7AAD (prepared in 1× binding buffer).

### CC3 Staining (Apoptotic Cells):

Fix sperm in 4% formaldehyde (diluted in 1× binding buffer): add ˜100 ul to ˜100 ul of sperm (final concentration: 2%). Let dry and keep at 4° C. until immunofluorescence with CC3 Ab is performed.

### BAI-TSR-GST (PS+ Cells)

1. incubate sperm with BAI-TSR 30 min 37 C/5% CO2 in TYH BSA.

2. fix with 2% PFA 15 min.

3. place sperm on slides smears.

4. immunofluorescence for GST (anti-GST Ab biotinylated).

5. Streptavidin with fluorescent.

### Buffers/Mediums

### 10× Binding Buffer:

### 0.1M Hepes (pH:7.4), 1.4M NaCl, 25 mM CaCl₂

### TYH Medium:

Add BSA: 4 mg/ml on day 0

### TH Medium

| Component | Conc. (mM) | MW | g/100 mL |
|---|---|---|---|
| NaCl | 119.3 | 58.44 | 0.6972 |
| KCl | 4.7 | 74.56 | 0.0350 |
| CaCl₂•2H₂O | 1.71 | 147.02 | 0.0251 |
| KH₂PO₄ | 1.2 | 136.09 | 0.0163 |
| MgSO₄•7H₂O | 1.2 | 246.48 | 0.0296 |
| NaHCO₃ | 25.1 | 84.01 | 0.2109 |
| Glucose | 5.56 | 180.16 | 0.1002 |
| Sodium pyruvate | 0.51 | 110.04 | 0.0056 |
| [symbol] | 10 μg/mL | | 100 μL |
| Penicilin G | | | 0.0075 |
| Streptomycin | | | 0.005 |
| [symbol] | 0.0006% | | 60 μL |
| Phenot red (0.5%) | | | 40 μL |

Filter-sterilize. Store at 4° C. Before using, add 4 mg/mL BSA (AlbuMax I, Gibco catalog #11020-021).
Modification from original protocol (similar to TYH + MBDC)

### Example 8

### Selecting Fertilization Competent Sperm

The presently disclosed subject matter shows that PS on sperm is involved in how the sperm enters the oocyte. Blockade of PS on sperm abrogates fertilization. Thus, PS+ sperm is the fertilization competent sperm, the ‘good sperm’. In some embodiments, magnetic microbead technology is used to select for the PS+ that can then be used in ART This technique is shown elsewhere herein using mouse sperm.

Briefly, washed spermatozoa are incubated with Annexin V-conjugated microbeads (Miltenyi Biotec) at room temperature for 15 min. 10 μl of microbeads are used per one million sperm cells. The spermatozoa-microbeads suspension are loaded on a column containing a coated cell-friendly matrix containing iron balls, which was fitted in a magnet (MiniMACS; Miltenyi Biotec). The power of magnetic field is measured as 0.5 Tesla between the poles and up to 1.5 Tesla within the iron globes of the column. The fraction containing the PS+ sperm (due to binding to Annexin V beads) are retained in the column, and then the sperm are eluted with a Ca⁺⁺ free buffer. As the binding of Annexin V to PS is Ca⁺⁺ dependent, the Ca⁺⁺ free buffer releases the PS+ sperm from the Annexin microbeads. The purified sperm are ready to use in IVF or other assisted reproductive techniques (ARTs). Such experiments have been performed with similarly purified mouse sperm and this procedure retains its fertilization potency.

Alternatively, another approach for selecting the PS+ sperm is by flow cytometry (FCM)/fluorescence-activated cell sorting (FACS). In this case sperm cells are labeled with Annexin V as described by the Positive Sperm staining protocol disclosed above and FACS performed.

### Example 9

### Functional Assessment of Sperm Cells

Myoblast-sperm fusion protocol:

Day 0: plate C2C12 myoblasts

In 4 well-permanox slides, plate ˜30.000 myoblasts in 0.5 ml of growth medium (GM). GM: 20% FBS+1% PSF in DMEM.

Day 1: Myoblast-sperm fusion

Bring Calcein-AM to room temperature and reconstitute with 12.66 ul DMSO (final concentration: 5 mM)

1. Obtain Sperm: Mouse sperm: Cut cauda epidydimis and let swim for 20 min in TYH+BSA or TYH+MBCD. Human sperm: enrich sperm using isolate gradient. Bull sperm: thaw straws in TYH+BSA medium.

2. Collect sperm in 15 ml tubes with PBS (add sperm to PBS) and centrifuge for 6 min at 1400 rpm. Collect the supernatant (carefully) in another 15 ml tube and centrifuge this tube.

3. Resuspend pellet in ˜0.5 ml PBS.

4. Label sperm with Calcein-AM (both “pellet” and “supernatant”). Add ˜0.4 ul of calcein to ˜0.5 ml of sperm suspension.

5. Incubate for 30 min, room temperature, dark.

6. Wash: Fill tube with PBS and centrifuge for 6 min at 1400 rpm.

7. Remove supernatant slowly with pipette and resuspend pellet in ˜0.5 ml GM.

8. Count #sperm:

9. Add 0.25×10⁶ sperm/well.

10. Co-culture sperm and myoblasts for 4 hours

11. Wash with PBS, stain with Hoechst (1:8000) for 5 min and wash with PBS again.

12. Mount.

For human sperm: stain myoblasts with Hoechst before adding the sperm because the sperm is very bright for Hoechst, which can make it difficult to take good pictures.

### REFERENCES

All references listed in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
1. Okabe, M. Sperm-egg interaction and fertilization: past, present, and future. Biol Reprod (2018).
2. Bianchi, E. & Wright, G. J. Sperm Meets Egg: The Genetics of Mammalian Fertilization. Annual review of genetics 50, 93-111 (2016).
3. Bianchi, E., Doe, B., Goulding, D. & Wright, G. J. Juno is the egg Izumo receptor and is essential for mammalian fertilization. Nature 508, 483-487 (2014).
4. Inoue, N., Ikawa, M., Isotani, A. & Okabe, M. The immunoglobulin superfamily protein Izumo is required for sperm to fuse with eggs. Nature 434, 234-238 (2005).
5. Ohto, U. et al. Structure of IZUMO1-JUNO reveals sperm-oocyte recognition during mammalian fertilization. Nature 534, 566-569 (2016).
6. Aydin, H., Sultana, A., Li, S., Thavalingam, A. & Lee, J. E. Molecular architecture of the human sperm IZUMO1 and egg JUNO fertilization complex. Nature 534, 562-565 (2016).
7. Inoue, N. et al. Molecular dissection of IZUMO1, a sperm protein essential for sperm-egg fusion. Development 140, 3221-3229 (2013).
8. Miyado, K. et al. Requirement of CD9 on the egg plasma membrane for fertilization. Science 287, 321-324 (2000).
9. Runge, K. E. et al. Oocyte CD9 is enriched on the microvillar membrane and required for normal microvillar shape and distribution. Dev Biol 304, 317-325 (2007).
10. Jankovicova, J., Simon, M., Antalikova, J., Cupperova, P. & Michalkova, K. Role of tetraspanin CD9 molecule in fertilization of mammals. Physiological research 64, 279-293 (2015).
11. Elliott, M. R. et al. Unexpected requirement for ELMO1 in clearance of apoptotic germ cells in vivo. Nature 467, 333-337 (2010).
12. Martin, G., Sabido, O., Durand, P. & Levy, R. Phosphatidylserine externalization in human sperm induced by calcium ionophore A23187: relationship with apoptosis, membrane scrambling and the acrosome reaction. Hum Reprod 20, 3459-3468 (2005).
13. de Vries, K. J., Wiedmer, T., Sims, P. J. & Gadella, B. M. Caspase-independent exposure of aminophospholipids and tyrosine phosphorylation in bicarbonate responsive human sperm cells. Biol Reprod 68, 2122-2134 (2003).
14. Gadella, B. M. & Harrison, R. A. Capacitation induces cyclic adenosine 3′,5′-monophosphate-dependent, but apoptosis-unrelated, exposure of aminophospholipids at the apical head plasma membrane of boar sperm cells. Biol Reprod 67, 340-350 (2002).
15. Hichri, R. et al. Apoptotic sperm biomarkers and the correlation between conventional sperm parameters and clinical characteristics. Andrologia (2017).
16. Sessions, A. & Horwitz, A. F. Myoblast aminophospholipid asymmetry differs from that of fibroblasts. FEBS Lett 134, 75-78 (1981).
17. Marguet, D., Luciani, M. F., Moynault, A., Williamson, P. & Chimini, G. Engulfment of apoptotic cells involves the redistribution of membrane phosphatidylserine on phagocyte and prey. Nat Cell Biol 1, 454-456 (1999).
18. Dillon, S. R., Mancini, M., Rosen, A. & Schlissel, M. S. Annexin V binds to viable B cells and colocalizes with a marker of lipid rafts upon B cell receptor activation. J Immunol 164, 1322-1332 (2000).
19. van den Eijnde, S. M. et al. Transient expression of phosphatidylserine at cell-cell contact areas is required for myotube formation. J Cell Sci 114, 3631-3642 (2001).
20. Hoppe, P. C. Fertilizing ability of mouse sperm from different epididymal regions and after washing and centrifugation. J Exp Zool 192, 219-222 (1975).
21. Ikawa, M., Inoue, N., Benham, A. M. & Okabe, M. Fertilization: a sperm's journey to and interaction with the oocyte. J Clin Invest 120, 984-994 (2010).
22. Richard, A. S. et al. Virion-associated phosphatidylethanolamine promotes TIM1-mediated infection by Ebola, dengue, and West Nile viruses. Proc Natl Acad Sci USA 112, 14682-14687 (2015).
23. Park, D. et al. BAI1 is an engulfment receptor for apoptotic cells upstream of the ELMO/Dock180/Rac module. Nature 450, 430-434 (2007).
24. Fadok, V. A., Warner, M. L., Bratton, D. L. & Henson, P. M. CD36 is required for phagocytosis of apoptotic cells by human macrophages that use either a phosphatidylserine receptor or the vitronectin receptor (alpha v beta 3). J Immunol 161, 6250-6257 (1998).
25. Hochreiter-Hufford, A. E. et al. Phosphatidylserine receptor BAI1 and apoptotic cells as new promoters of myoblast fusion. Nature 497, 263-267 (2013).
26. Lemke, G. Phosphatidylserine Is the Signal for TAM Receptors and Their Ligands. Trends Biochem Sci 42, 738-748 (2017).
27. Stanton, J. L. & Green, D. P. A set of 840 mouse oocyte genes with well-matched human homologues. Mol Hum Reprod 7, 521-543 (2001).
28. Hamann, J. et al. International Union of Basic and Clinical Pharmacology. XCIV. Adhesion G protein-coupled receptors. Pharmacol Rev 67, 338-367 (2015).
29. Nishimori, H. et al. A novel brain-specific p53-target gene, BAI1, containing thrombospondin type 1 repeats inhibits experimental angiogenesis. Oncogene 15, 2145-2150 (1997).
30. Zhu, D. et al. BAI1 regulates spatial learning and synaptic plasticity in the hippocampus. J Clin Invest 125, 1497-1508 (2015).
31. Bolliger, M. F., Martinelli, D. C. & Sudhof, T. C. The cell-adhesion G protein-coupled receptor BAI3 is a high-affinity receptor for C1q-like proteins. Proc Natl Acad Sci USA 108, 2534-2539 (2011).
32. Sigoillot, S. M. et al. The Secreted Protein C1QL1 and Its Receptor BAI3 Control the Synaptic Connectivity of Excitatory Inputs Converging on Cerebellar Purkinje Cells. Cell Rep (2015).
33. Tait, J. F. & Smith, C. Phosphatidylserine receptors: role of CD36 in binding of anionic phospholipid vesicles to monocytic cells. The Journal of biological chemistry 274, 3048-3054 (1999).
34. Greenberg, M. E. et al. Oxidized phosphatidylserine-CD36 interactions play an essential role in macrophage-dependent phagocytosis of apoptotic cells. J Exp Med 203, 2613-2625 (2006).
35. Silverstein, R. L. & Febbraio, M. CD36, a scavenger receptor involved in immunity, metabolism, angiogenesis, and behavior. Sci Signal 2, re3 (2009).
36. Klenotic, P. A. et al. Histidine-rich glycoprotein modulates the anti-angiogenic effects of vasculostatin. The American journal of pathology 176, 2039-2050 (2010).
37. Johnson, M. H., Eager, D., Muggleton-Harris, A. & Grave, H. M. Mosaicism in organisation concanavalin A receptors on surface membrane of mouse egg. Nature 257, 321-322 (1975).
38. Rothlin, C. V., Carrera-Silva, E. A., Bosurgi, L. & Ghosh, S. TAM receptor signaling in immune homeostasis. Annu Rev Immunol 33, 355-391 (2015).
39. Kim, K., Lee, S E, Kee, K A Expression of Gas6 receptors, Tyro3, Axl, and Mertk, in oocytes and embryos and effects of Mertk RNAi on the oocyte maturation. Dev Reprod 16, 195-204 (2012).
40. Ravichandran, K. S. & Lorenz, U. Engulfment of apoptotic cells: signals for a good meal. Nat Rev Immunol 7, 964-974 (2007).
41. Gregory, C. D. & Pound, J. D. Cell death in the neighbourhood: direct microenvironmental effects of apoptosis in normal and neoplastic tissues. J Pathol 223, 177-194 (2011).
42. Conover, J. C. & Gwatkin, R. B. Pre-loading of mouse oocytes with DNA-specific fluorochrome (Hoechst 33342) permits rapid detection of sperm-oocyte fusion. J Reprod Fertil 82, 681-690 (1988).
43. Ohnami, N. et al. CD81 and CD9 work independently as extracellular components upon fusion of sperm and oocyte. Biol Open 1, 640-647 (2012).
44. Ravaux, B., Garroum, N., Perez, E., Willaime, H. & Gourier, C. A specific flagellum beating mode for inducing fusion in mammalian fertilization and kinetics of sperm internalization. Sci Rep 6, 31886 (2016).
45. Ravichandran, K. S. Find-me and eat-me signals in apoptotic cell clearance: progress and conundrums. J Exp Med 207, 1807-1817 (2010).
46. Zagorska, A., Traves, P. G., Lew, E. D., Dransfield, I. & Lemke, G. Diversification of TAM receptor tyrosine kinase function. Nat Immunol 15, 920-928 (2014).
47. Yanagihashi, Y., Segawa, K., Maeda, R., Nabeshima, Y. I. & Nagata, S. Mouse macrophages show different requirements for phosphatidylserine receptor Tim4 in efferocytosis. Proc Natl Acad Sci USA 114, 8800-8805 (2017).
48. Penberthy, K. K. et al. Context-dependent compensation among phosphatidylserine-recognition receptors. Sci Rep 7, 14623 (2017).
49. Hamoud, N., Tran, V., Croteau, L. P., Kania, A. & Cote, J. F. G-protein coupled receptor BAI3 promotes myoblast fusion in vertebrates. Proc Natl Acad Sci USA 111, 3745-3750 (2014).
50. Geisbrecht, E. R. et al. Drosophila ELMO/CED-12 interacts with Myoblast city to direct myoblast fusion and ommatidial organization. Dev Biol 314, 137-149 (2008).
51. Gumienny, T. L. et al. CED-12/ELMO, a novel member of the CrkII/Dock180/Rac pathway, is required for phagocytosis and cell migration. Cell 107, 27-41 (2001).
52. Brugnera, E. et al. Unconventional Rac-GEF activity is mediated through the Dock180-ELMO complex. Nat Cell Biol 4, 574-582 (2002).
53. Gallardo, T., Shirley, L., John, G. B. & Castrillon, D. H. Generation of a germ cell-specific mouse transgenic Cre line, Vasa-Cre. Genesis 45, 413-417 (2007).
54. Halet, G. & Carroll, J. Rac activity is polarized and regulates meiotic spindle stability and anchoring in mammalian oocytes. Dev Cell 12, 309-317 (2007).
55. Jeong, J. & Conboy, I. M. Phosphatidylserine directly and positively regulates fusion of myoblasts into myotubes. Biochem Biophys Res Commun 414, 9-13 (2011).
56. Charrin, S. et al. Normal muscle regeneration requires tight control of muscle cell fusion by tetraspanins CD9 and CD81. Nature communications 4, 1674 (2013).
57. Tachibana, I. & Hemler, M. E. Role of transmembrane 4 superfamily (TM4SF) proteins CD9 and CD81 in muscle cell fusion and myotube maintenance. J Cell Biol 146, 893-904 (1999).
58. Park, S. Y., Yun, Y. & Kim, I. S. CD36 is required for myoblast fusion during myogenic differentiation. Biochem Biophys Res Commun 427, 705-710 (2012).
59. Vasyutina, E., Martarelli, B., Brakebusch, C., Wende, H. & Birchmeier, C. The small G-proteins Rac1 and Cdc42 are essential for myoblast fusion in the mouse. Proc Natl Acad Sci USA 106, 8935-8940 (2009).
60. Mattioli, M., Gloria, A., Mauro, A., Gioia, L. & Barboni, B. Fusion as the result of sperm-somatic cell interaction. Reproduction 138, 679-687 (2009).
61. Bendich, A., Borenfreund, E. & Sternberg, S. S. Penetration of somatic mammalian cells by sperm. Science 183, 857-859 (1974).
62. Kaji, K. et al. The gamete fusion process is defective in eggs of Cd9-deficient mice. Nat Genet 24, 279-282 (2000).
63. Arikketh, D., Nelson, R. & Vance, J. E. Defining the importance of phosphatidylserine synthase-1 (PSS1): unexpected viability of PSS1-deficient mice. The Journal of biological chemistry 283, 12888-12897 (2008).
64. Segawa, K. & Nagata, S. An Apoptotic ‘Eat Me’ Signal: Phosphatidylserine Exposure. Trends in cell biology 25, 639-650 (2015).
65. Inoue, N. & Wada, I. Monitoring dimeric status of IZUMO1 during the acrosome reaction in living spermatozoon. Cell Cycle 17, 1279-1285 (2018).
66. Naito, K., Fukuda, Y. & Toyoda, Y. Effects of porcine follicular fluid on male pronucleus formation in porcine oocytes matured in vitro. Gamete Res 21, 289-295 (1988).
67. Guan, M. et al. In vitro fertilization in mice using the MBCD-GSH protocol. Curr Protoc Mouse Biol 4, 67-83 (2014).
68. Nagy, A. Manipulating the mouse embryo: a laboratory manual, Edn. 3rd. (Cold Spring Harbor Laboratory Press, Cold Spring Harbor, N. Y.; 2003).
69. Enwere, E. K. et al. TWEAK and cIAP1 regulate myoblast fusion through the noncanonical NF-kappaB signaling pathway. Sci Signal 5, ra75 (2012).
70. Millay, D. P. et al. Myomaker is a membrane activator of myoblast fusion and muscle formation. Nature 499, 301-305 (2013).

It will be understood that various details of the presently disclosed subject matter can be changed without departing from the scope of the presently disclosed subject matter. Furthermore, the foregoing description is for the purpose of illustration only, and not for the purpose of limitation.

1. A method of detecting fertilization competent sperm cells in a sample, the method comprising:
(a) providing a sample comprising sperm cells from a subject;
(b) mixing the sample with a reagent that selectively binds phosphatidylserine (PS) on the sperm cells; and
(c) detecting living sperm cells bound to the reagent in the sample, whereby fertilization-competent sperm cells are detected.

2-9. (canceled)

10. A method of isolating fertilization competent sperm, the method comprising:
(a) providing a sample comprising sperm cells from a subject;
(b) mixing the sample with a reagent that selectively and/or reversibly binds phosphatidylserine (PS) on the sperm cells; and
(c) isolating sperm cells bound to the reagent that selectively and/or reversibly binds PS, whereby fertilization competent sperm are isolated.

11-66. (canceled)

67. A method of assessing fusion competency in sperm cells, the method comprising
(a) providing sperm cells from a subject;
(b) labeling sperm with a reagent that can be subsequently tracked to assess fusion;
(c) adding the sperm cells to a culture comprising fusion competent cells; and
(d) assessing fusion competency of the sperm cells by detecting the label transfer to fusion competent cells in the culture.

68. The method of claim 67, wherein the subject is a human subject or a non-human animal subject.

69. The method of claim 68, wherein the non-human animal subject is selected from the group consisting of bovine, equine, porcine, ovine, canine, feline, and avian.

70. The method of claim 67, wherein the sample is formed by concentrating a semen sample from the subject.

71. The method of claim 67, wherein the reagent that can be subsequently tracked to assess fusion comprises a calcein or a Dil dye.

72. The method of claim 67, further comprising adding a contrast agent to the culture.

73. The method of claim 72, wherein the contrast agent comprises a Hoechst counterstaining agent.

74. The method of claim 67, wherein the culture comprises about 10,000 to about 50,000 fusion competent cells.

75. A kit for detecting and/or isolating fertilization competent sperm cells in a sample, the kit comprising: a reagent that selectively and/or reversibly binds phosphatidylserine (PS); and instructional material for detecting and/or isolating fertilization-competent sperm cells in a sample, optionally wherein the reagent that selectively and/or reversibly binds PS also reversibly binds PS.

76. A kit for assessing fusion competency in sperm cells, the kit comprising a reagent that can be subsequently tracked to assess fusion; a culture comprising fusion competent cells; and instructional material for assessing fusion competency in the sperm cells by detecting the reagent in the culture.

77. The kit of claim 76, comprising a reagent for concentrating sperm cells in a sample.

78. The kit of claim 76, wherein the reagent that can be subsequently tracked to assess fusion comprises a calcein or a DiI dye.

79. The kit of claim 76, further comprising a contrast agent for the culture.

80. The kit of claim 79, wherein the contrast agent comprises a Hoechst counterstaining agent.

81. The kit of claim 76, wherein the culture comprises about 10,000 to about 50,000 fusion competent cells.

82. A method of blocking fertilization, the method comprising providing a reagent that blocks PS binding between a sperm cell and an oocyte, and administering a composition comprising the reagent topically to a subject at a time of sexual intercourse.

83. A contraceptive device comprising a coating or lubricant comprising a reagent that blocks PS binding between a sperm cell and an oocyte.

84. A contraceptive composition comprising a reagent that blocks PS binding between a sperm cell and an oocyte.

85. A method of detecting a fertilization competent oocyte in a sample, the method comprising:
(a) providing a sample comprising an oocyte from a subject;
(b) mixing the sample with a reagent that selectively binds at least one phosphatidylserine (PS) receptor on the oocyte; and
(c) detecting an oocyte bound to the reagent in the sample, whereby a fertilization-competent oocyte is detected.


SEQUENCE LISTING (2 sequences)

SEQ ID NO: 1 - protein, 218 aa, Homo sapiens
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPK

SEQ ID NO: 2 - protein, 384 aa, Homo sapiens
SSHPCGIMQTPCACLGGDVGDPASSPLVPRGDVCLRDGVAGGPENCLTSLTQDRGGHGSAGGWKLWSLWGECTRDCGGGLQTRTRTCLPTLGVEGGGCEGVLEEGRLCNRKACGPTGRSSSRSQSLRSTDARRREEFGDELQQFGFPSPQTGDPAAEEWSPWSVCSSTCGEGWQTRTRFCVSSSYSTQCSGPLREQRLCNNSAVCPVHGAWDEWSPWSLCSSTCGRGFRDRTRTCRPPQFGGNPCEGPEKQTKFCNIALCPGRAVDGNWNEWSSWSTCSASCSQGRQQRTRECNGPSYGGAECQGHWVETRDCFLQQCPVDGKWQAWASWGSCSVTCGGGSQRRERVCSGPFFGGAACQGPQDEYRQCGAQRCPEPHEICDEDN